(19) United States
(12) Patent Application Publication
(10) Pub. No.: US 2025/0361529 A1
(43) Pub. Date: Nov. 27, 2025

(54) GENE EDITING SYSTEM FOR TREATING DUCHENNE MUSCULAR DYSTROPHY, AND METHOD OF TREATING DISEASE USING SAME

(71) Applicant: GENKORE INC., Daejeon (KR)

(72) Inventors: Yong Sam KIM, Seoul (KR); Do Yon KIM, Daejeon (KR); Jeong Mi LEE, Daejeon (KR); Hyun Jung CHIN, Daejeon (KR)

(73) Assignee: GENKORE INC., Daejeon (KR)

(21) Appl. No.: 18/845,108

(22) PCT Filed: Mar. 10, 2023
(86) PCT No.: PCT/KR2023/003337

§ 371 Date: May 9, 2025

(30) Foreign Application Priority Data
Mar. 10, 2022 (KR) 10-2022-0030000
May 27, 2022 (KR) 10-2022-0065600

(51) Int. Cl. C12N 15/90; C12N 9/22; C12N 15/11; C12N 15/113

(52) U.S. Cl. CPC C12N 15/907; C12N 9/226; C12N 15/11; C12N 15/113; C12N 2310/14; C12N 2310/20; C12N 2310/531; C12N 2320/33

(57) ABSTRACT

A gene editing system for treating Duchenne muscular dystrophy, and a method for treating the disease using the gene editing system are disclosed. The system and method have the effects of making it possible to package the gene editing system in a single vector by editing the dystrophin gene using a CRISPR/Cas12f1 or TaRGET system, as well as making it possible to produce the dystrophin protein having a normal function by preventing the production of a stop codon of exon 51 through the skipping of exon 51, and thus can be useful for treating Duchenne muscular dystrophy.

### TECHNICAL FIELD

This application claims priorities based on Korean Patent Application No. 2022-0030000, filed on Mar. 10, 2022, and Korean Patent Application No. 2022-0065600, filed on May 27, 2022, the entire disclosures of which are incorporated herein by reference.

The present disclosure relates to a gene editing system for treating Duchenne muscular dystrophy and a method for treating a disease using the same. Specifically, the present disclosure relates to a CRISPR/Cas12f1 gene editing system (for example, TaRGET system) for treating Duchenne muscular dystrophy and a method for treating a disease using the same. In addition, the present disclosure relates to a CRISPR/Cas12f1 gene editing system (for example, TaRGET system) and a method for deleting a nucleic acid segment comprising exon 51 in a dystrophin gene.

### BACKGROUND ART

Duchenne muscular dystrophy (DMD) is a disease that involves progressive weakening of striated muscle due to an abnormality in the dystrophin gene on the X chromosome. It is usually diagnosed around 3 to 5 years of age when motor development is accelerated, and it may also be discovered in the asymptomatic period due to an increase in serum creatine kinase level. Duchenne muscular dystrophy leaves children unable to walk by an average age of 9.5 years, and no later than 13 years. A mild form of the disease, in which walking is possible until beyond an age of 16 years, is classified as Becker muscular dystrophy (BMD). DMD occurs in approximately one out of every 5,000 male births, and has the highest number of patients among muscular dystrophy disorders.

Unlike other genetic diseases, 80% of mutations in the DMD disease are caused by deletion or duplication of one or several exons. In particular, deletion/duplication of a specific exon or point mutation therein causes structural abnormalities of dystroglycan (dystrophin-binding glycoprotein) and severe functional impairment in muscle tissue.

Meanwhile, in a case where the deleted exon consists of a multiple of three nucleotides and does not disrupt the reading frame, only the amino acid sequence information of the deleted exon portion is lost, and most of the amino acid sequence is conserved identical to that of the normal dystrophin. In other words, although it is challenging to restore the deleted exon in the dystrophin gene through gene correction, it may be therapeutically effective to create, through gene correction, additional deletions around an abnormal gene so that the amino acid sequence followed by the abnormal gene is restored. Through gene correction that creates additional deletions around an abnormal gene to skip the abnormal gene, known as exon skipping, it is possible to induce a protein that is partially truncated and still functional despite a genetic mutation.

In correcting dystrophin gene abnormalities, the treatable patient population varies depending on the targeted exon. Exon 51 skipping, which can be applied to the largest number of patients, can treat 14% of all DMD patients and 20% of patients with deletion mutations. In addition, skipping of exon 53 can treat 10% of all patients and 15% of patients with deletion mutations. Besides, skipping of exon 45 (9% of all patients and 13% of patients with deletion mutations), exon 44 (7% of all patients and 11% of patients with deletion mutations), exon 43 (7% of all patients and 11% of patients with deletion mutations), exon 46 (5% of all patients and 7% of patients with deletion mutations), exon 50 (4% of all patients and 6% of patients with deletion mutations), exon 52 (4% of all patients and 5% of patients with deletion mutations), exon 55 (3% of all patients and 4% of patients with deletion mutations), or exon 8 (2% of all patients and 3% of patients with deletion mutations) can be used for treatment.

Gene therapy through exon skipping has been studied by several companies, and some DMD therapeutics have received FDA approval. Exondys 51 (active ingredient: eteplirsen), Vyondys 53 (active ingredient: golodirsen), and Amondys 45 (active ingredient: casimersen), which have been developed by Sarepta Therapeutics, are RNA-based therapeutics that target exon 51, exon 53, and exon 45, respectively. Each of these therapeutics allows the antisense oligonucleotide to bind to the exon in question, thereby preventing expression of the protein encoded by the exon. However, RNA-based therapeutics often show only temporary treatment rather than sustained therapeutic effects, and also frequently cause adverse effects. Exondys 51 was approved by the FDA in 2016, and showed efficacy in only 13% of the target patient population. Vyondys 53 received accelerated approval from the FDA in 2019, and cases of renal toxicity, including fatal glomerulonephritis, have been observed therefor following administration of the antisense oligonucleotide.

Recently, with advancements in CRISPR genetic scissors technology, studies are being attempted to apply it to development of DMD therapeutics. However, the CRISPR technology has problems such as low intracellular gene editing activity or difficulty in packaging a gene editing system into a single vector. Therefore, it is necessary to develop a technology that can increase intracellular gene editing activity or package the gene editing system into a single vector and deliver the same in order to treat DMD.

### DISCLOSURE OF INVENTION

### Technical Problem

The object of the present disclosure is to solve the above-mentioned problems of the prior art.

An object of the present disclosure is to provide a CRISPR/Cas12f1 editing technique for deleting a nucleic acid segment comprising exon 51 in a dystrophin gene.

Another object of the present disclosure is to provide a method for treating or delaying onset or progression of Duchenne muscular dystrophy using the CRISPR/Cas12f1 editing technique.

The object of the present disclosure is not limited to the above-mentioned objects. The objects of the present disclosure will become clearer from the following description and may be realized by means and combinations thereof as set forth in the claims.

### Solution to Problem

Representative configurations of the present disclosure to achieve the above-mentioned objects are as follows.

According to an aspect of the present disclosure, there is provided an editing system for a dystrophin gene, comprising an endonuclease comprising Cas12f1 or a variant protein thereof, or a nucleic acid encoding the endonuclease; an engineered guide RNA comprising a first guide sequence that hybridizes to a target sequence in a dystrophin gene, or a nucleic acid encoding the guide RNA; and an engineered guide RNA comprising a second guide sequence that hybridizes to a target sequence in a dystrophin gene, or a nucleic acid encoding the guide RNA, wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

According to another aspect of the present disclosure, there is provided a composition, comprising an endonuclease comprising Cas12f1 or a variant protein thereof, or a nucleic acid encoding the endonuclease; an engineered guide RNA comprising a first guide sequence that hybridizes to a target sequence in a dystrophin gene, or a nucleic acid encoding the guide RNA; and an engineered guide RNA comprising a second guide sequence that hybridizes to a target sequence in a dystrophin gene, or a nucleic acid encoding the guide RNA, wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

According to yet another aspect of the present disclosure, there is provided a vector system, comprising an endonuclease comprising Cas12f1 or a variant protein thereof, or a nucleic acid encoding the endonuclease; an engineered guide RNA comprising a first guide sequence that hybridizes to a target sequence in a dystrophin gene, or a nucleic acid encoding the guide RNA; and an engineered guide RNA comprising a second guide sequence that hybridizes to a target sequence in a dystrophin gene, or a nucleic acid encoding the guide RNA, wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

According to still yet another aspect of the present disclosure, there is provided an engineered guide RNA, comprising a spacer region, which comprises a guide sequence capable of hybridizing to a target sequence in a dystrophin gene, and a scaffold region, wherein the guide sequence is capable of hybridizing to a target sequence of contiguous 15 to 30 bp in length, wherein the target sequence is adjacent to the 5′-end or the 3′-end of a protospacer-adjacent motif (PAM) sequence which is present in a region 5000 bp upstream and downstream of dystrophin exon 51 and is recognized by Cas12f1 or a variant protein thereof.

According to still yet another aspect of the present disclosure, there is provided a virus produced by the vector system disclosed herein.

According to still yet another aspect of the present disclosure, there is provided a method for deleting a segment comprising exon 51 in a dystrophin gene in a cell, comprising bringing into contact with the cell the system, composition, or vector system disclosed herein.

### Advantageous Effects of Invention

Duchenne muscular dystrophy, which is caused by abnormalities in a dystrophin gene, can be treated by a therapeutic strategy that deletes exon 51 in the dystrophin gene and thus allows for production of a protein that can function normally. The present inventors have found that a more efficient and broadly applicable gene editing system, which comprises Cas12f1 protein (for example, UnCas12f1, CWCas12f1, or a variant protein thereof) as a novel hypercompact nucleic acid cleavage protein and an engineered guide RNA whose specific regions are modified to exhibit superior indel efficiency when used in combination with the protein, can effectively delete exon 51. The system has excellent nucleic acid cleavage efficiency (especially, double-strand cleavage efficiency) for a target nucleic acid or target gene. The Cas12f1 protein is about ⅓ smaller in molecular weight than existing nucleic acid degrading proteins including the Cas9 protein, which has been studied the most to date, and the engineered guide RNA is also much smaller than the wild-type Cas12f1 guide RNA. Thus, the system has the advantage of being deliverable in vivo even with a vector (for example, adeno-associated virus (AAV) vector) having a limited capacity for the size of genes to be carried. The small-sized gene editing system allows for in vivo delivery using a single vector along with other components that can increase deletion efficiency of exon 51. In addition, the present inventors have surprisingly confirmed the fact that the deletion efficiency of exon 51 can be increased by inhibiting expression of a gene involved in non-homologous end joining (NHEJ).

The present inventors have developed a novel hypercompact nucleic acid editing system that allows for in vivo delivery using a single vector and enables effective large deletion of exon 51 in the dystrophin gene.

### BRIEF DESCRIPTION OF DRAWINGS

FIG. 1 illustrates a schematic diagram of a therapeutic method for Duchenne muscular dystrophy using the system according to an embodiment.

FIG. 2 illustrates modification sites in the engineered guide RNA of the system according to an embodiment.

FIG. 3 illustrates human codon-optimized amino acid sequences of UnCas12f1 (SEQ ID NO: 5), CWCas12f1 (TnpB) (SEQ ID NO: 1) and variant proteins thereof (SEQ ID NOs: 2 to 4) (FIGS. 3A to 3C), and human codon-optimized nucleotide sequences encoding such proteins (FIGS. 3D to 3H).

FIG. 4 illustrates a graph showing deletion efficiency of exon 51, depending on candidate gRNAs of the system according to an embodiment and combinations thereof, in HEK293 cells.

FIG. 5 illustrates a graph showing deletion efficiency of exon 51, depending on candidate gRNAs of the system according to an embodiment and combinations thereof, in AC16 cells.

FIG. 6 illustrates a graph showing deletion efficiency of exon 51, depending on promoters used in the Cas12f1 or TaRGET system according to an embodiment, in HEK293 cells. The experiments using UNCas12f1 protein are indicated by “Cas12f1,” and the experiments using CWCas12f1 protein is indicated by “TaRGET.”

FIG. 7 illustrates graphs obtained by measuring mRNA expression levels of ATM1 and XRCC4 in cells transfected with shRNAs according to an embodiment.

FIG. 8 illustrates graphs obtained by measuring mRNA expression levels of XLF-1 and XRCC6 in cells transfected with shRNAs according to an embodiment.

FIG. 9 illustrates graphs obtained by measuring mRNA expression levels of LIG4 and DCLRE1C in cells transfected with shRNAs according to an embodiment.

FIG. 10 illustrates a graph obtained by identifying, with qRT-PCR, deletion efficiency of exon 51 achieved by inhibited expression of NHEJ-related genes in HEK293 cells. The experiments using UNCas12f1 protein are indicated by “Cas12f1,” and the experiments using CWCas12f1 protein are indicated by “TaRGET.”

FIG. 11 illustrates a graph obtained by identifying, with qRT-PCR, deletion efficiency of exon 51 achieved by inhibited expression of NHEJ-related genes in AC16 cells. The experiments using UNCas12f1 protein are indicated by “Cas12f1,” and the experiments using CWCas12f1 protein are indicated by “TaRGET.”

FIG. 12 illustrates a graph obtained by identifying, with qRT-PCR, deletion efficiency of exon 51 using one shRNA or a combination of two or more shRNAs in HEK293 cells. The experiments using UNCas12f1 protein are indicated by “Cas12f1,” and the experiments using CWCas12f1 protein are indicated by “TaRGET.”

FIG. 13 illustrates a graph obtained by identifying, with qRT-PCR, deletion efficiency of exon 51 using one shRNA or a combination of two or more shRNAs in AC16 cells. The experiments using UNCas12f1 protein are indicated by “Cas12f1,” and the experiments using CWCas12f1 protein are indicated by “TaRGET.”

FIG. 14 illustrates a graph obtained by identifying, with qRT-PCR, deletion efficiency of exon 51 depending on the number of days post transfection using the systems comprising shRNA according to an embodiment. Depending on the CRISPR proteins used therein, the systems are indicated by SaCas9, Cas12f1 (UNCas12f1), and TaRGET (CWCas12f1), respectively.

FIG. 15 illustrates a graph obtained by identifying, with qRT-PCR, deletion efficiency of exon 51 depending on promoters used in the systems comprising shRNA according to an embodiment.

### MODES FOR CARRYING OUT INVENTION

The detailed description to be described later of the present disclosure will be described with reference to specific drawings (wherever such drawings exist) with respect to specific embodiments in which the present disclosure may be practiced; however, the present disclosure is not limited thereto and, if properly described, is limited only by the appended claims, along with the full scope of equivalents to which such claims are entitled. It should be understood that various embodiments/examples of the present disclosure, although different, are not necessarily mutually exclusive. For example, a particular feature, structure, or characteristic described herein may be changed from one embodiment/example to another embodiment/example or implemented in combinations of embodiments/examples without departing from the technical spirit and scope of the present disclosure. Unless defined otherwise, technical and scientific terms used herein have the same meanings as generally used in the art to which the present disclosure belongs. For purposes of interpreting this specification, the following definitions will apply and, whenever appropriate, terms used in the singular will also include the plural and vice versa.

Hereinafter, in order to enable a person having ordinary skill in the art to easily practice the present disclosure, various preferred embodiments/examples of the present disclosure will be described in detail with reference to the attached drawings (wherever such drawings exist).

### Definition

As used herein, “nucleic acid,” “nucleotide,” “nucleoside,” and “base” have the meanings commonly understood by a person skilled in the art. Specifically, “nucleic acid” is a biological molecule composed of nucleotides, and is used interchangeably with polynucleotide. The nucleic acid comprises both DNA and RNA which are single-stranded or double-stranded. “Nucleotide” is a unit composed of phosphoric acid, a pentose sugar, and a base (or nucleobase). In RNA (ribonucleic acid), the pentose sugar is ribose, and in DNA (deoxyribonucleic acid), the pentose sugar is deoxyribose. The nucleotide has one selected from adenine (A), guanine (G), cytosine (C), thymine (T), and uracil (U) as a nucleobase. Adenine, guanine, and cytosine exist both in RNA and DNA, thymine exists only in DNA, and uracil exists only in RNA. In addition, the pentose sugar and nucleobase constituting the nucleotide may be referred to as “nucleoside.” The nucleoside is classified into adenosine, thymidine, cytidine, guanosine, and uridine according to the type of nucleobase. The abbreviations for base, nucleoside, and nucleotide may be identical and may be appropriately interpreted depending on the context. For example, the sequence 5′-UUUUU-3′ may be a sequence of five consecutive bases (uracil), a sequence of five consecutive nucleosides (uridine), and/or a sequence of five consecutive nucleotides (uridine monophosphate). In addition, when describing a nucleic acid, RNA, and DNA, nucleotides constituting the nucleic acid, RNA, and DNA are abbreviated as uridine, adenosine, thymidine, cytidine, and guanosine according to the type of nucleoside. The above abbreviation may be appropriately interpreted depending on the context. For example, RNA comprising a sequence of four consecutive uridine residues may be interpreted as RNA comprising four consecutive uridine monophosphate nucleotides. In addition, the terms nucleic acid, nucleotide, nucleoside, and base used herein may include modified nucleic acids, nucleotides, nucleosides, and bases known in the art for improving, for example, their safety or immunogenicity.

As used herein, “target nucleic acid” or “target gene” refers to a nucleic acid or gene that is subjected to gene editing (for example, double-strand cleavages or deletion of gene segments) or targeted by a gene editing system (for example, a Cas12f1 system or a TaRGET system). These terms may be used interchangeably and refer to the same subject. Unless otherwise defined, the target gene may be a unique gene or nucleic acid possessed by a target cell (for example, a prokaryotic cell, a eukaryotic cell, an animal cell, a mammalian cell, or a plant cell), a gene or nucleic acid of external origin, or an artificially synthesized nucleic acid or gene, and may mean single-stranded or double-stranded DNA or RNA. The target gene or target nucleic acid may be a mutant gene involved in a genetic disease. In an embodiment, the target gene or target nucleic acid may be a human dystrophin gene. In an embodiment, the target gene or target nucleic acid may be a mutant human dystrophin gene.

As used herein, “target region” means a region of a target gene to which a guide RNA is designed to bind and cleave. The target region may comprise a target sequence. In addition, in double-stranded nucleic acids, the target region may refer to a region that comprises a target sequence (included in the target strand) and a sequence complementary thereto (included in the non-target strand).

As used herein, “target sequence” refers to a sequence located in a target nucleic acid or a target gene, which is recognized by a guide RNA, or a sequence to be modified by the CRISPR/Cas12f1 system or TaRGET system. Specifically, the target sequence refers to a sequence complementary to a guide sequence included in a guide RNA or a sequence that complementarily binds to a guide sequence. The strand comprising the target sequence is referred to as a “target strand.” When the target nucleic acid or the target gene is single-stranded, the strand may be the target strand. When the target nucleic acid or the target gene is double-stranded, one of the double strands may be a target strand, and a strand complementary to the target strand may exist. The strand complementary to the target strand is referred to as a “non-target strand.” The “non-target strand” comprises a PAM (Protospacer Adjacent Motif) sequence and a protospacer sequence. The PAM sequence is a sequence recognized by Cas12f1 or a variant protein thereof in the CRISPR/Cas12f1 system or the TaRGET system. The protospacer sequence, which is located at the 5′-end or the 3′-end of the PAM sequence, is a sequence having complementarity to a target sequence or a sequence that forms a complementary bond with a target sequence. Correlation between the protospacer sequence and the target sequence is similar to correlation between the target sequence and the guide sequence. Due to these characteristics, a guide sequence may be designed using a protospacer sequence. That is, a guide sequence which complementarily binds to a target sequence may be designed as a nucleotide sequence having the same nucleotide sequence as the protospacer sequence, and the guide sequence is designed by replacing T in the protospacer sequence with U.

As used herein, “stem” refers to a nucleic acid region having a secondary structure that comprises a nucleotide region capable of forming a double strand. A configuration in which a double strand is connected primarily by a region of single-stranded nucleotides (a loop region) is referred to as a “stem-loop.” “Stem” and “stem-loop” may be used interchangeably and should be interpreted appropriately depending on the context.

The terms “nuclease” and “endonuclease” refer to enzymes that possess catalytic activity for DNA cleavage and may be used interchangeably.

The term “non-homologous end joining (NHEJ) DNA repair pathway” refers to a mechanism that repairs a double-strand break in a nucleotide sequence by direct ligation of the broken ends without the requirement for a homologous template (as opposed to homology-directed repair (HDR), which requires a homologous sequence to induce healing of a double-strand break in a nucleotide sequence). NHEJ often leads to loss (deletion) of a nucleotide sequence near the double-strand break site.

The term “vector,” unless otherwise specified, refers to any material capable of transporting a genetic material into a cell. For example, a vector may be a nucleic acid, typically a DNA molecule, comprising a genetic material of interest, for example, a nucleic acid encoding an effector protein (Cas protein) of a CRISPR/Cas system, and/or a nucleic acid encoding a guide RNA; however, the vector is not limited thereto.

The term “operably linked” means a functional linkage of two or more components arranged in such a way that allows the described component to function in an intended manner. For example, when a promoter sequence is operably linked to a sequence encoding protein A, it means that the promoter is linked to the sequence encoding the protein A so as to transcribe and/or express the sequence encoding the protein A in a cell. In addition, the term includes all other meanings generally recognized by those skilled in the art and may be appropriately interpreted depending on the context.

The term “engineered” is used to distinguish a substance or molecule from one having a naturally occurring configuration, and means that the substance or molecule is obtained by application of artificial modification. For example, “engineered guide RNA” refers to a guide RNA obtained by applying an artificial modification to the configuration of a naturally occurring guide RNA.

The term “NLS (nuclear localization sequence or signal)” refers to an amino acid sequence that promotes introduction of a substance from outside the nucleus into the nucleus, for example, by nuclear transport. The term “NES (nuclear export sequence or signal)” refers to an amino acid sequence that promotes transport of a substance from inside the nucleus to the outside of the nucleus, for example, by nuclear transport. The terms NLS or NES are known in the relevant art and may be clearly understood by those skilled in the art.

The term “about” refers to an amount, level, value, number, frequency, percent, dimension, size, amount, weight, or length that varies by approximately 30, 25, 20, 15, 10, 9, 8, 7, 6, 5, 4, 3, 2, or 1% with respect to a reference amount, level, value, number, frequency, percent, dimension, size, amount, weight, or length. For example, the term “about” may mean x±5% when used in relation to the value x expressed as a number or numerical value.

The term “subject” is used interchangeably with “patient” and may be a mammal in need of prevention or treatment of Duchenne muscular dystrophy, such as primate (for example, human), companion animal (for example, dog and cat), domestic animal (for example, cow, pig, horse, sheep, and goat), or laboratory animal (for example, rat, mouse, and guinea pig). In an embodiment of the present disclosure, the subject is a human.

The term “treatment” generally refers to obtaining a desired pharmacological and/or physiological effect. Such an effect has a therapeutic effect in that it partially or completely cures a disease and/or harmful effects caused by the disease. Desirable therapeutic effects include, but are not limited to, prevention of occurrence or recurrence of a disease, improvement of symptoms, reduction of any direct or indirect pathological consequences of a disease, prevention of metastasis, reduction of disease progression rate, improvement or alleviation of disease state, and remission or improved prognosis. Preferably, “treatment” may be deletion of a segment comprising exon 51 in the dystrophin gene or restoration of the reading frame of the dystrophin gene caused thereby.

The term “target nucleic acid editing system,” “gene editing system,” or “gene restoration system” as used herein refers to a system that comprises a nucleic acid degrading enzyme, such as nucleic acid editing protein or endonuclease, and a nucleic acid-targeting molecule corresponding to the nucleic acid degrading enzyme, and this system binds to or interacts with a target nucleic acid or target gene so that a target region of the target nucleic acid or target gene can be cleaved, edited, repaired, and/or restored. Here, the nucleic acid-targeting molecule may be represented by an engineered guide RNA (gRNA), but is not limited thereto. Meanwhile, the target nucleic acid editing system may exist in any form capable of editing the target nucleic acid. For example, the system may be in a form of a composition that comprises a complex comprising a nucleic acid degrading enzyme and a nucleic acid-targeting molecule, may be in a form of a kit in which the nucleic acid degrading enzyme and the nucleic acid-targeting molecule are each included in separate compositions, or may be a vector system or composition comprising at least one vector that comprises a nucleic acid encoding the nucleic acid degrading enzyme and a nucleic acid encoding the nucleic acid-targeting molecule.

The term “hypercompact TaRGET system” refers to a gene editing system that comprises a nucleic acid degrading enzyme such as hypercompact CRISPR/Cas protein or tiny endonuclease (for example, Cas12f1 or a variant thereof) and a nucleic acid-targeting molecule corresponding to the nucleic acid degrading enzyme, and is used for differentiation from the existing gene editing system. Here, the nucleic acid-targeting molecule may be represented by an engineered guide RNA (gRNA), but is not limited thereto. The system may be any type of gene editing system capable of binding to a target nucleic acid or target gene so that a target region of the target nucleic acid or gene is cleaved, edited, repaired, and/or restored.

The term “endonuclease” may be used interchangeably with “nucleic acid editing protein,” “gene editing protein,” or “nucleic acid degrading protein.” The molecule referred to as this endonuclease or protein refers to a (endo-) nuclease that recognizes the targeting nucleic acid, DNA or RNA, or a protospacer adjacent motif (PAM) present in a target gene, and then allows double-strand breaks (DSBs) to occur at nucleotide sequences within or outside the target nucleotide sequence. In addition, the endonuclease, the nucleic acid editing protein, or the like is also referred to as an effector protein that constitutes a nucleic acid construct for a nucleic acid editing system or homology directed repair. Here, the effector protein may be a nucleic acid degrading protein capable of binding to a guide RNA (gRNA) or engineered gRNA, or may be a peptide fragment capable of binding to a target nucleic acid or target gene.

The term “guide RNA (gRNA)” refers RNA that is capable of forming a complex with a molecule referred to as an endonuclease, a gene editing protein, a nucleic acid degrading protein, or the like, and interacting with (for example, hybridizing to, forming a complementary bond(s) with, or forming a hydrogen bond(s) with) a target nucleotide sequence, and comprises a guide sequence having sufficient complementarity with the target nucleotide sequence to cause sequence-specific binding of the complex to the target nucleotide sequence. In the present disclosure, a guide RNA and a guide molecule may be used interchangeably.

The terms “tracrRNA (trans-activating crRNA)” and “crRNA (CRISPR RNA)” have the meanings generally understood by those skilled in the art in the field of gene editing technology. These terms may be used to refer to respective molecules of a dual guide RNA found in nature, and may also be used to refer to respective portions of a single guide RNA (sgRNA) in which the tracrRNA and the crRNA are connected by a linker. Unless otherwise stated, the description tracrRNA and crRNA simply means tracrRNA and crRNA that constitute a guide RNA.

The term “scaffold region” refers collectively to a portion of a guide RNA (gRNA) which can interact with a molecule called endonuclease, gene editing protein, nucleic acid degrading protein, or the like, and may be used to refer to the remaining portion of a guide RNA found in nature, excluding a spacer.

The terms “guide sequence,” “spacer,” or “spacer sequence” may be used interchangeably and refer to a polynucleotide within the CRISPR/Cas system which is capable of interacting with (for example, hybridizing to, forming a complementary bond(s) with, or forming a hydrogen bond(s) with) a target sequence portion. For example, the guide sequence or spacer sequence refers to 10 to 50 consecutive nucleotides linked directly or indirectly through a linker or the like to or near the 3′-end of crRNA, which constitutes a guide RNA, in a target nucleic acid editing system.

The term “engineered” may be used interchangeably with “non-naturally occurring,” “artificial,” or “modified,” and means something that is not in its natural form, state, or the like as found in nature. In a case where the term indicates a guide RNA, a guide polynucleotide, or a nucleic acid molecule, the guide RNA, the guide polynucleotide, or the nucleic acid molecule is meant to be substantially free of at least one component that is found in nature or naturally occurring, or to substantially contain at least one component that is not found in nature or non-naturally occurring. For example, the “engineered guide RNA” refers to gRNA obtained by applying artificial modification to a configuration (for example, sequence) of a guide RNA (gRNA) that exists in nature, and may be referred to herein as an “augmented RNA.”

The term “wild-type” is a term of art understood by those skilled in the art and means a typical form of an organism, strain, gene, protein, or characteristic as it occurs in nature to the extent that it is distinguishable from mutant or variant forms.

The term “variant” should be understood to mean expression of qualities having a pattern that deviates from what occurs in nature. For example, when referring to Cas12f1 or a variant protein thereof, the variant protein may mean a variant of (wild-type) Cas12f1.

The term “nucleic acid construct” refers to a structure that comprises, as components, a nucleotide sequence encoding an endonuclease, a nucleic acid editing protein, a nucleic acid degrading protein, or the like and/or a nucleotide sequence encoding a guide RNA, and if necessary, may further comprise nucleotide sequences encoding various types of (poly)peptides or linkers.

The terms “protein,” “polypeptide,” and “peptide” may be used interchangeably and refer to a polymeric form of amino acids of any length which may comprise genetically coded and non-genetically coded amino acids, chemically or biochemically modified or derivatized amino acids, and polypeptides having modified peptide backbones. The terms include fusion proteins, including, but not limited to, fusion proteins with a heterologous amino acid sequence, fusions with heterologous and homologous leader sequences, with or without N-terminal methionine residues, immunologically tagged proteins, and the like.

All technical terms used in the present disclosure, unless otherwise defined, have meanings commonly understood by those skilled in the relevant technical field and may be interpreted appropriately depending on the context.

### II. Dystrophin Gene

Dystrophin refers to a rod-shaped cytoplasmic protein that is part of a protein complex which links the cytoskeleton of muscle fibers to the surrounding extracellular matrix through the cell membrane. Dystrophin maintains muscle cell integrity and provides structural stability to the cell membrane. Dystrophin is expressed by the dystrophin gene, which is 2.4 megabases in size and contains 79 exons at the genetic locus Xp21, and consists of more than 3,600 amino acid residues.

Duchenne muscular dystrophy is caused by hereditary or spontaneous mutations in the dystrophin gene (for example, human dystrophin gene) (that is, mutations that result in nonsense or frameshift mutations in the dystrophin gene). Naturally occurring mutations associated with Duchenne muscular dystrophy and consequences thereof are well known. In-frame deletions occurring in a region of exons 45 to 55 (for example, exon 51) may produce a functional dystrophin protein, and individuals who carry such mutations have no symptoms or exhibit only mild symptoms. In patients with Duchenne muscular dystrophy, it is possible to delete non-essential exons (for example, exon 51) so that the disrupted reading frame of the dystrophin gene is restored, thereby producing a dystrophin protein that, although partially deleted, is functional. Gene editing of exon 51 in the dystrophin gene (for example, deletion or removal of a segment comprising exon 51) may be considered for treating Duchenne muscular dystrophy or delaying its onset or progression.

As disclosed herein, mutations in the dystrophin gene may be corrected by gene editing, such as by deleting a segment comprising exon 51 in the dystrophin gene using the Cas12f1 system of the present disclosure.

As disclosed herein, editing of dystrophin gene (for example, deletion of exon 51 in the dystrophin gene using the Cas12f1 system of the present disclosure) may be used to treat Duchenne muscular dystrophy (for example, Duchenne muscular dystrophy caused by mutations in exon 51 of the dystrophin gene) or delay onset or progression thereof.

In an embodiment, editing of dystrophin gene may be deletion of a segment comprising exon 51 (that is, skipping of exon 51). The Cas12f1 system for editing the dystrophin gene (for example, deleting a segment comprising exon 51), or the like is described in detail below.

### III. CRISPR/Cas System for Editing Dystrophin Gene

As disclosed herein, a CRISPR/Cas12f1 system for editing or modifying a dystrophin gene (for example, a human dystrophin gene) is provided. The disclosed system comprises (i) an endonuclease comprising at least one Cas12f1 protein or a variant thereof or a nucleic acid encoding the endonuclease and (ii) at least one (for example, two) guide RNA molecule or a nucleic acid encoding the same.

The present inventors have confirmed that TnpB (Transposon-associated transposase B) protein derived from the Candidatus Woesearchaeota archaeon has an amino acid sequence similar to UnCas12f1 protein (and thus, TnpB with an amino acid sequence similar to UnCas12f1 protein is also named CWCas12f1; CWCas12f1 may be collectively referred to as Cas12f1 protein together with UnCas12f1, and may belong to a variant of Cas12f1 in its relationship with UnCas12f1), has a molecular weight that is about ⅓ smaller than that of an existing nucleic acid degrading protein including the Cas9 protein, which has been studied the most to date, and has a significantly higher nucleic acid cleavage efficiency for a target nucleic acid or target gene. In addition, the present inventors have confirmed that engineered guide RNAs having a small size obtained by modifying the wild-type Cas12f1 guide RNA may induce excellent nucleic acid cleavage efficiency (for example, a double-strand break) together with the Cas12f1 protein such as CWCas12f1 or UnCas12f1. The hypercompact gene editing system comprising an engineered guide RNA and Cas12f1 or a variant thereof, such as CWCas12f1 or UnCas12f1, disclosed herein may be referred to as “Cas12f1 system” or “TaRGET system,” and these terms may be used interchangeably. (However, in the examples, for convenience, the system using the UnCas12f1 protein is referred to as Cas12f1 system, and the system using the CWCas12f1 protein is referred to as TaRGET system).

The gene editing system of the present disclosure is capable of generating at least one cleavage (for example, single-strand break or double-strand break) near a target site of the dystrophin gene (for example, a region upstream or downstream of exon 51, or both). The at least one cleavage may be made outside the target sequence or inside of the 3′-end (for example, 1 to 5 bp inside thereof).

In an embodiment, the CRISPR/Cas12f1 system may comprise two or more guide RNAs that target different sequences in the dystrophin gene. The target sequences may overlap with each other.

In another embodiment, the guide RNA may target a region adjacent to exon 51 in the dystrophin gene to generate cleavage (for example, single-strand break or double-strand break).

In yet another embodiment, the two guide RNAs may target regions upstream and downstream of exon 51 in the dystrophin gene, respectively, to generate at least one cleavage (for example, two single-strand breaks or two double-strand breaks).

In still yet another embodiment, at least two guide RNAs may be used to generate at least two sets of cleavage (for example, two single-strand breaks, one double-strand break, and one single-strand break; or two pairs of single-strand breaks).

For example, the system disclosed herein may induce deletion of a nucleic acid segment comprising exon 51 by generating cleavages with two guide RNA molecules, each targeting a region upstream or downstream of exon 51, together with a Cas12f1 endonuclease.

In another embodiment, in the system disclosed herein, the endonuclease comprising Cas12f1 or a variant protein thereof and the guide RNA may be included in the form of a ribonucleoprotein particle (RNP). Respective components of the Cas12f1 system are described below.

### 1. Endonuclease Comprising Cas12f1 or Variant Protein Thereof

The gene editing system based on CRISPR/Cas12f1 of the present disclosure comprises an endonuclease comprising Cas12f1 or a variant thereof or a nucleic acid encoding the endonuclease. Cas12f1 or a variant thereof is a (small) endonuclease characterized by exhibiting excellent activity in cleaving a target site of a target nucleic acid and being significantly smaller in size by about ⅓ of the nucleic acid degrading protein than the existing CRISPR/Cas9 system.

Cas12f1 is one of the effector proteins named Cas14 in a previous study (see Harrington et al., Science, 362, 839-842, 2018), and is also called Cas14a1 protein. The Cas12f1 protein disclosed herein may be a wild-type Cas12f1 protein that exists in nature. In addition, the Cas12f1 protein may be a variant of the wild-type Cas12f1 protein. The variant of Cas12f1 is referred to as a “Cas12f1 variant.” The Cas12f1 variant may be a variant having the same or equivalent function as the wild-type Cas12f1 protein, a variant of which some or all functions are modified, and/or a variant in which additional functions are added. In addition, the Cas12f1 protein may be an engineered form of the wild-type Cas12f1 protein. It may be engineered to alter or improve the function of the wild-type Cas12f1 protein. It may be used interchangeably with the Cas12f1 variant.

It has been reported that the Cas12f1 protein forms a complex with a guide RNA such that two Cas12f1 protein molecules bind, in the form of a dimer, to a guide RNA, and that all or part of the domain of the Cas12f1 protein recognizes a specific part of the scaffold region of the Cas12f1 guide RNA to form a CRISPR/Cas12f1 complex (see Takeda et al., Structure of the miniature type V-F CRISPR-Cas effector enzyme, Molecular Cell 81, 1-13, 2021; and Xiao et al., Structural basis for the dimerization-dependent CRISPR-Cas12f nuclease, bioRxiv, 2020). Cas12f1 protein or a variant thereof may generate a double-strand or single-strand break in a target nucleic acid or a target gene. Deletion of a desired gene segment may be induced by such a double-strand or single-strand break.

The Cas12f1 protein may recognize a PAM sequence located in a target nucleic acid or target gene. The PAM sequence is a unique sequence that is determined depending on the CRISPR protein. The PAM sequence recognized by Cas12f1 may be a T-rich sequence. The PAM sequence recognized by Cas12f1 may be a 5′-TTTR-3′sequence, wherein R may be T, A, C, or G. Preferably, the PAM sequence may be 5′-TTTA-3′, 5′-TTTT-3′, 5′-TTTC-3′, or 5′-TTTG-3′. More preferably, the PAM sequence may be 5′-TTTA-3′ or 5′-TTTG-3′.

In an embodiment, the Cas12f1 protein may be derived from a Cas14 family (see Harrington et al., Science 362, 839-842 (2018); US 2020/0172886 A1).

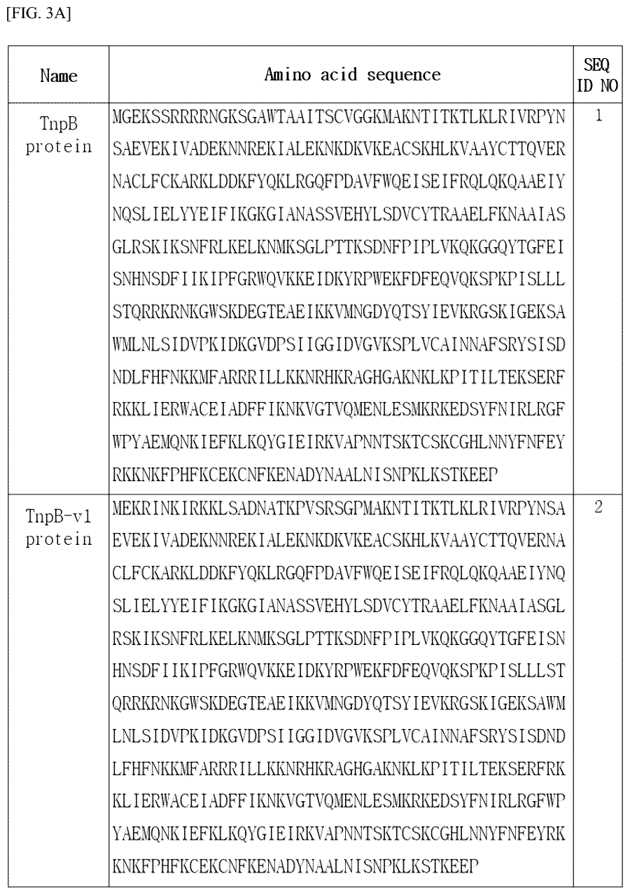

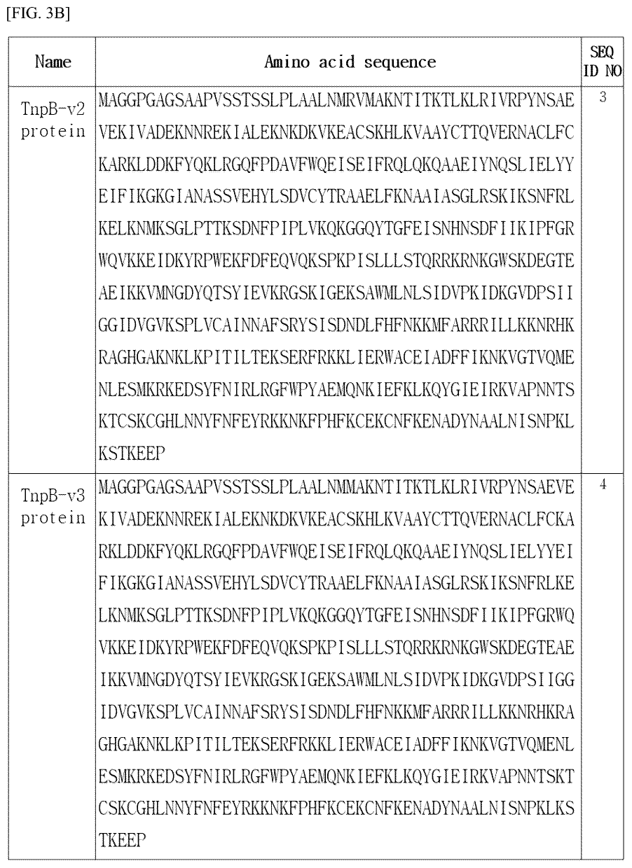

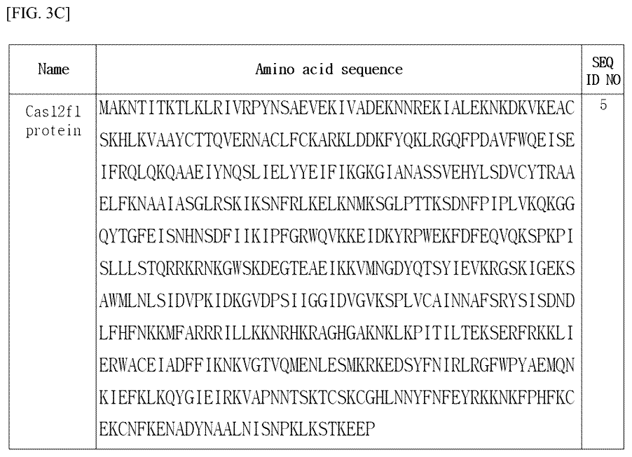

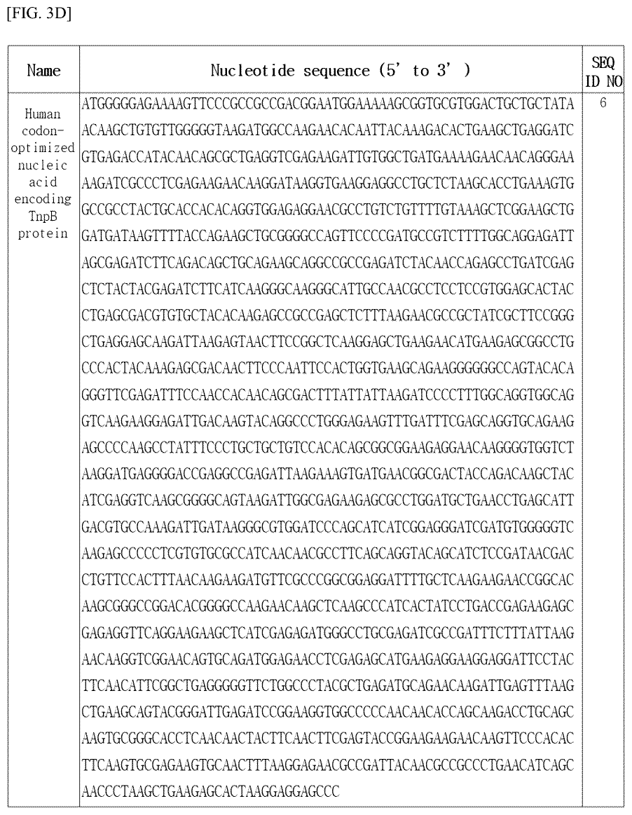

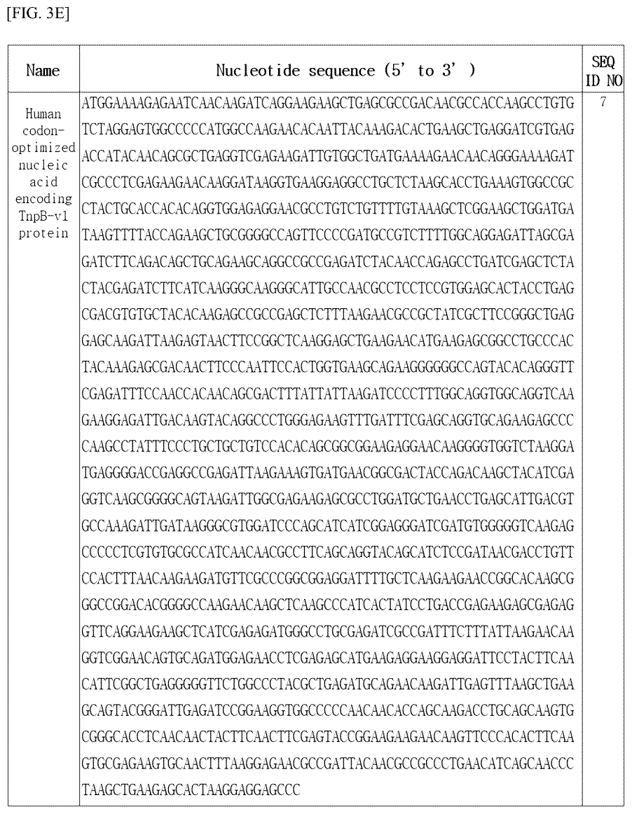

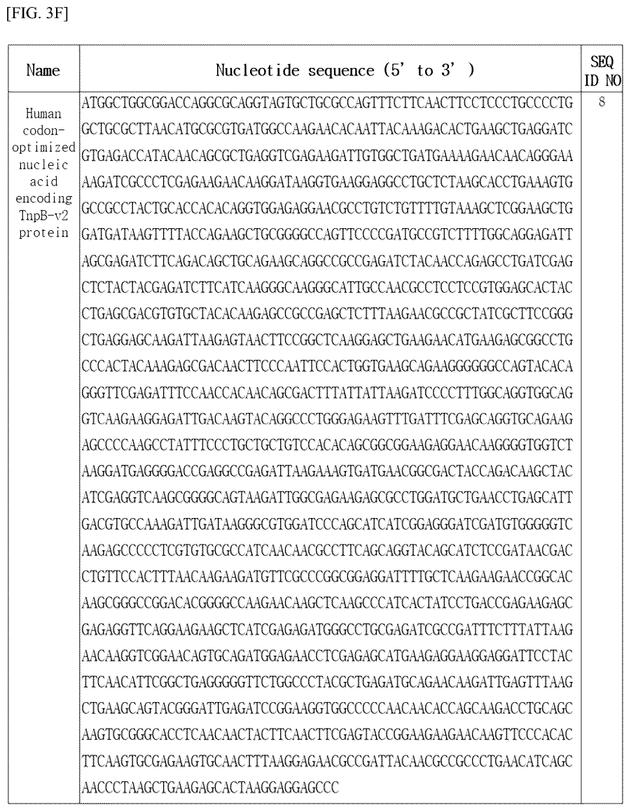

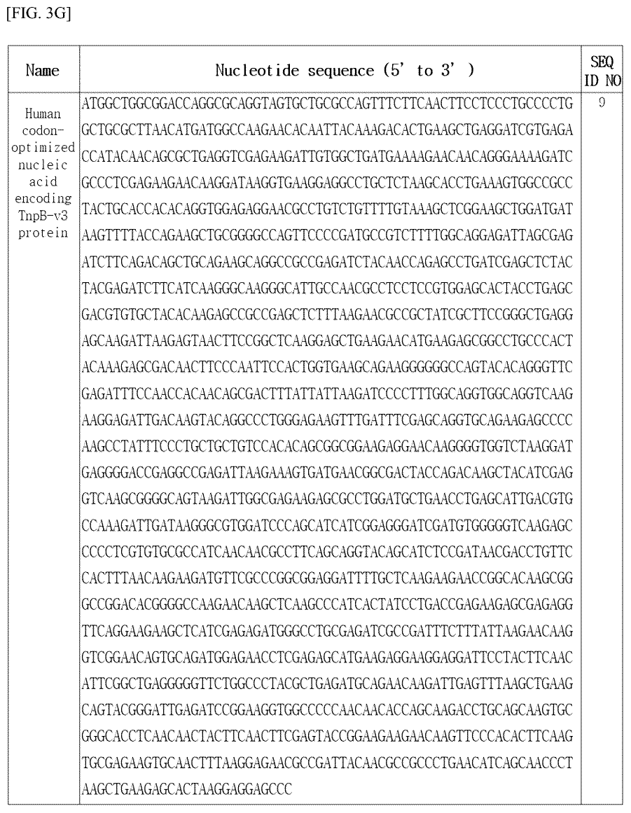

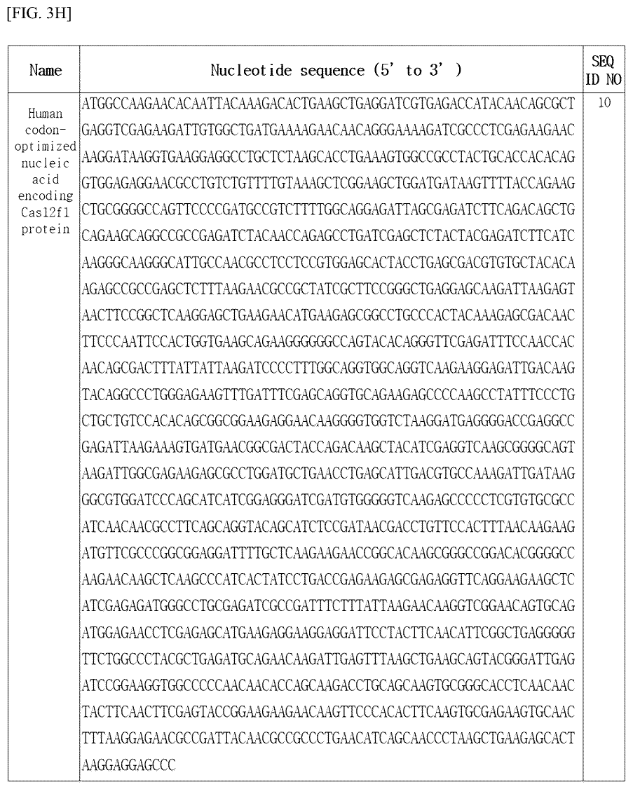

In another embodiment, the Cas12f1 protein may be Cas14a1 (UnCas12f1) protein derived from an uncultured archaeon (see Harrington et al., Science 362, 839-842 (2018); US 2020/0172886 A1). For example, the UnCas12f1 protein may comprise or consist of the amino acid sequence of SEQ ID NO: 5 (see FIG. 3).

In yet another embodiment, the Cas12f1 protein may be TnpB (transposon-associated transposase B) protein derived from the Candidatus Woesearchaeota archaeon. The TnpB protein is a protein conventionally known as a transposase. To date, the TnpB protein has been known only as a transposon-encoded nuclease, and it is not known whether the TnpB protein has Cas endonuclease activity. In addition, the guide RNA for the TnpB protein has also not been known. The present inventors have confirmed for the first time that TnpB variant or engineered TnpB, which is based on the TnpB protein sequence, has excellent endonuclease activity of targeting a target nucleic acid or a target gene and cleaving a double-stranded DNA of the target site while having a similar size to a Cas12f1 protein, which belongs to the group with the smallest molecular weight among nucleic acid degrading proteins, and have constructed an engineered guide RNA that exhibits excellent gene editing activity when used together with TnpB or a variant protein thereof. The TnpB protein derived from Candidatus Woesearchaeota archaeon is also referred to as CWCas12f1. For example, the CWCas12f1 protein may comprise or consist of the amino acid sequence of SEQ ID NO: 1 (see FIG. 3).

In an embodiment, the Cas12f1 protein may be a Cas12f1 variant. The Cas12f1 variant may comprise a modification of at least one amino acid, such as deletion, substitution, insertion, or addition, compared to the amino acid sequence of the wild-type Cas12f1 protein.

In another embodiment, the Cas12f1 variant may comprise deletion of at least one amino acid or substitution with another amino acid sequence compared to the amino acid sequence of the wild-type Cas12f1 protein (for example, the amino acid sequence of RuvC domain or PAM recognition domain).

In yet another embodiment, the Cas12f1 variant may be a variant having at least one amino acid sequence added to the N-terminus and/or C-terminus of the amino acid sequence of wild-type Cas12f1 (for example, UnCas12f1 or CWCas12f1) or a variant protein thereof. The present inventors have confirmed that among the variants having amino acids added to the N-terminus and/or C-terminus of the wild-type Cas12f1 protein, there are variants having a function equivalent to the wild-type Cas12f1. For this purpose, reference may be made to Korean Patent Application No. 10-2021-0181875, the entire disclosure of which should be deemed to be incorporated herein. Preferably, the Cas12f1 variant may be such that it has 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids added to the N-terminus and/or C-terminus of wild-type Cas12f1 or a variant protein thereof. In an embodiment, Cas12f1 (for example, UnCas12f1 or CWCas12f1) or a variant protein thereof may comprise an amino acid sequence having the amino acid sequence of SEQ ID NO: 1 in which 1 to 28 amino acids at the N-terminus are removed or substituted. For example, the Cas12f1 variant may comprise or consist of TnpB-v1 protein (SEQ ID NO: 2), which further comprises 26 amino acids derived from the N-terminus of CasX at the N-terminus of the UnCas12f1 protein, TnpB-v2 protein (SEQ ID NO: 3), which further comprises 28 random amino acid sequences at the N-terminus of the UnCas12f1 protein, or TnpB-v3 protein (SEQ ID NO: 4), which further comprises 26 random amino acid sequences at the N-terminus of the UnCas12f1 protein (see FIG. 3).

In an embodiment, the Cas12f1 variant may be such that it is engineered to recognize a PAM sequence other than 5′-TTTA-3′ or 5′-TTTG-3′. In an embodiment, the Cas12f1 variant may comprise substitution of at least one amino acid residue selected from the group consisting of amino acids at position 170 (serine), position 174 (tyrosine), position 184 (alanine), position 188 (serine), position 191 (arginine), position 225 (glutamine), position 230 (tyrosine), position 271 (valine), and position 272 (glutamine) with respect to the wild-type sequence of CWCas12f1 (TnpB) (for example, amino acid sequence of SEQ ID NO: 1). Preferably, the Cas12f1 variant may comprise substitution of at least one amino acid residue selected from the group consisting of amino acids at position 170 (serine, S), position 188 (serine, S), position 191 (arginine, R), position 225 (glutamine, Q), and position 272 (glutamine, Q). More preferably, the Cas12f1 variant may comprise one or more selected from the following substitutions with respect to the wild-type sequence (for example, the amino acid sequence of SEQ ID NO: 1): S170T, S188Q, S188H, S188K, R191K, Q225T, Q225F, and Q272K (T: threonine, Q: glutamine, H: histidine, K: lysine, F: phenylalanine). Furthermore, the Cas12f1 variant may comprise an amino acid sequence selected from the group consisting of SEQ ID NOs: 392 to 399. These Cas12 variants may further recognize 5′-TNTN-3′, 5′-TTTN-3′, 5′-TGTA-3′, 5′-TCTG-3′, 5′-TGTG-3′, or 5′-TTTC-3′, wherein N is A, T, C, or G.

In another embodiment, the Cas12f1 variant may be a fusion protein. The fusion protein may comprise two or more heterologous polypeptide domains, wherein one polypeptide domain comprises a Cas12f1 protein or a variant thereof, and the other domain comprises a (poly)peptide having another function or activity. For example, the (poly)peptide having another function or activity may have methylase activity, demethylase activity, transcription activation activity, transcription repression activity, transcription release factor activity, histone modification activity, RNA cleavage activity, or nucleic acid binding activity. In addition, the (poly)peptide, which has a different function or activity, may be a tag or reporter protein for separation and/or purification. For example, the tag or reporter protein includes, but is not limited to, a tag protein such as a histidine (His) tag, a V5 tag, a FLAG tag, an influenza hemagglutinin (HA) tag, a Myc tag, a VSV-G tag, and a thioredoxin (Trx) tag; a fluorescent protein such as green fluorescent protein (GFP), yellow fluorescent protein (YFP), cyan fluorescent protein (CFP), blue fluorescent protein (BFP), HcRED, and DsRed; and a reporter protein (enzyme) such as glutathione-S-transferase (GST), horseradish peroxidase (HRP), chloramphenicol acetyltransferase (CAT), beta-galactosidase, beta-glucuronidase, and luciferase.

In addition, the (poly)peptide having another function or activity may be, but is not limited to, reverse transcriptase, deaminase or another proteolytic enzyme.

In another embodiment, the Cas12f1 or variant protein thereof may comprise an amino acid sequence having at least 70%, at least 75%, at least 80%, at least 85%, at least 90%, or at least 95% sequence identity to an amino acid sequence selected from the group consisting of SEQ ID NOs: 1 to 5.

In yet another embodiment, the Cas12f1 protein may comprise an amino acid sequence having at least 70%, at least 75%, at least 80%, at least 85%, at least 90%, or at least 95% sequence identity to the amino acid sequence of SEQ ID NO: 1 or 5.

In an embodiment, the Cas12f1 (or a variant thereof) protein may comprise one selected from the following sequences: (i) the amino acid sequence of SEQ ID NO: 5; (ii) the amino acid sequence of SEQ ID NO: 1; (iii) an amino acid sequence having the amino acid sequence of SEQ ID NO: 1 in which 1 to 28 amino acids at the N-terminus have been removed or substituted; or (iv) an amino acid sequence having the amino acid sequence of SEQ ID NO: 1 in which 1 to 600 amino acids have been added to the N-terminus or C-terminus.

In another embodiment, the Cas12f1 variant protein may be a protein comprising or consisting of one selected from amino acid sequences having the amino acid sequence of SEQ ID NO: 1 in which 1 to 600 amino acids added to the N-terminus or C-terminus. Here, there is no limitation on the added sequence of 1 to 600 amino acids. For example, the added 1 to 600 amino acids may be the amino acid sequence of SEQ ID NO: 390 or SEQ ID NO: 391. An NLS or NES sequence may further be included between the added sequence and the Cas12f1 variant protein.

In an embodiment, since the target nucleic acid editing system of the present disclosure involves cleaving a nucleic acid at a target site in a target nucleic acid or target gene, the target site may be located in the nucleus of a cell. The Cas12f1 or variant protein thereof may comprise one or more nuclear localization signal (NLS) sequences that localize the molecule into the nucleus. For example, the one or more nuclear localization signal sequences may have a sufficient amount or activity to induce the Cas12f1 or variant protein thereof to be targeted to the nucleus of a eukaryotic cell (for example, a mammalian cell) in a detectable amount. For example, differences in the strength of activity may result from the number of NLSs included in the Cas12f1 or variant protein thereof, the type of specific NLS(s) used, or a combination of these factors. For example, the NLS may be, but is not limited to, an NLS sequence derived from NLS of SV40 virus large T-antigen, NLS from nucleoplasmin, c-myc NLS, hRNPA1 M9 NLS, the sequence of IBB domain from importin-alpha, the sequence of myoma T protein, the sequence of human p53, the sequence of mouse c-abl IV, the sequence of influenza virus NS1, the sequence of hepatitis virus delta antigen, the sequence of mouse Mx1 protein, the sequence of human poly(ADP-ribose) polymerase, or the sequence of steroid hormone receptor (human) glucocorticoid.

In another embodiment, the Cas12f1 or variant protein thereof may comprise a nuclear export sequence (NES).

In another embodiment, the Cas12f1 or variant protein thereof may be a fusion with various enzymes that may be involved in a gene expression process within cells. Here, the Cas12f1 or variant protein thereof to which the enzymes are fused may cause various quantitative and/or qualitative changes in gene expression in cells. For example, the various enzymes to be additionally bound may be DNMT, TET, KRAB, DHAC, LSD, p300, Moloney Murine Leukemia Virus (M-MLV) reverse transcriptase, or variants thereof. The Cas12f1 or variant protein thereof to which the reverse transcriptase is fused may also function as a prime editor.

In an embodiment, there is provided a nucleic acid encoding Cas12f1 or a variant thereof. The nucleic acid encoding Cas12f1 or a variant thereof may be codon optimized for a subject (for example, a human) into which the Cas12f1 protein is to be introduced. For example, the human codon optimized nucleotide sequence encoding Cas12f1 or a variant thereof may be, for example, at least one selected from SEQ ID NOs: 6 to 10.

### 2. Guide RNA

As disclosed herein, the CRISPR/Cas12f1 system comprises at least one guide RNA or a nucleic acid encoding the guide RNA. The Cas12f1 guide RNA provides targeting for CRISPR/Cas12f1. The guide RNA of the CRISPR/Cas12f1 system of the present disclosure may be a Cas12f1 guide RNA found in nature or an engineered Cas12f1 guide RNA. The Cas12f1 guide RNA found in nature or engineered Cas12f1 guide RNA comprises a scaffold region and a spacer region. The scaffold region of the Cas12f1 guide RNA is a region that comprises parts of tracrRNA (trans-activating CRISPR RNA) and crRNA (CRISPR RNA) and functions to interact with the Cas12f1 protein. The spacer region of the Cas12f1 guide RNA comprises a guide sequence.

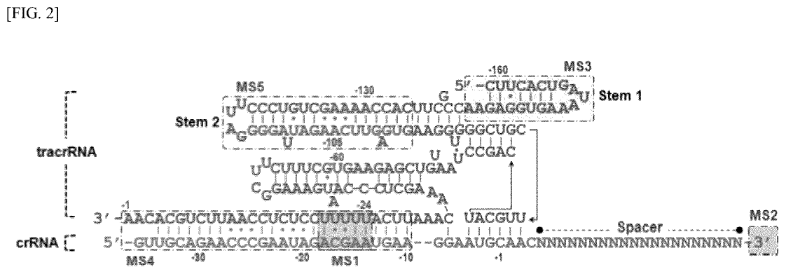

The wild-type guide RNA comprises two structures in which a part of tracrRNA (tracrRNA anti-repeat) and a part of crRNA repeat (crRNA repeat) are complementarily bound to form a duplex, which are conveniently referred to as R:AR1 and R:AR2 portions. The wild-type guide RNA may comprise (i) at least one stem region, (ii) a tracrRNA-crRNA complementarity region, and optionally (iii) a region comprising three or more consecutive uracil (U) residues. Specifically, the wild-type guide RNA may sequentially comprise, from the 5′-end, a first stem region, a second stem region, a third stem region, a fourth stem region, and a fifth stem region (a tracrRNA-crRNA complementarity region). For example, referring to FIG. 2, the scaffold region of the wild-type guide RNA comprises five stem regions, that is, a first stem region (stem 1), a second stem region (stem 2), a third stem region (stem 3), a fourth stem region (stem 4), and a fifth stem region (stem 5 (R:AR2)), from the 5′-end. The region comprising stem 5 (R:AR2) is also referred to as a tracrRNA-crRNA complementarity region.

More specifically, the wild-type gRNA may comprise a wild-type tracrRNA having the nucleotide sequence of SEQ ID NO: 11, or a wild-type crRNA having the nucleotide sequence of SEQ ID NO: 12. In addition, the wild-type gRNA may be fused in the form of a single guide RNA to become a single guide RNA (sgRNA) having the nucleotide sequence of SEQ ID NO: 13.

TABLE 1

| Name | Nucleotide sequence | SEQ ID NO |
|---|---|---|
| Wild-type | CUUCACUGAUAAAGUGGAGAACCGCUUCAC | 11 |
| tracrRNA | CAAAAGCUGUCCCUUAGGGGAUUAGAACUU | |
| | GAGUGAAGGUGGGCUGCUUGCAUCAGCCUA | |
| | AUGUCGAGAAGUGCUUUCUUCGGAAAGUAA | |
| | CCCUCGAAACAAAUUCAUUUUUCCUCUCCAA | |
| | UUCUGCACAA | |
| Wild-type | GUUGCAGAACCCGAAUAGACGAAUGAAGGA | 12 |
| crRNA | AUGCAAC | |
| Canonical | CUUCACUGAUAAAGUGGAGAACCGCUUCAC | 13 |
| sgRNA | CAAAAGCUGUCCCUUAGGGGAUUAGAACUU | |
| | GAGUGAAGGUGGGCUGCUUGCAUCAGCCUA | |
| | AUGUCGAGAAGUGCUUUCUUCGGAAAGUAA | |
| | CCCUCGAAACAAAUUCAUUUUUCCUCUCCAA | |
| | UUCUGCACAAgaaaGUUGCAGAACCCGAAUAG | |
| | acgaaUGAAGGAAUGCAACNNNNNNNNNNNNN | |
| | NNNNNNN | |

### 2.1. Guide Sequence

The guide RNA may comprise at least one guide sequence that hybridizes to a target sequence in the dystrophin gene. Since a protospacer sequence complementary to the target sequence is located at the 5′- or 3′-end of the PAM sequence recognized by the Cas12f1 protein, the guide sequence may be designed using the protospacer sequence. The guide sequence, which binds complementarily to the target sequence, may be designed as a nucleotide sequence having the same nucleotide sequence as the protospacer sequence. When the protospacer sequence is a DNA sequence, the guide sequence may be such that T in the protospacer sequence is replaced with U.

In an embodiment, the guide sequence may be hybridizable or complementary to a target sequence of contiguous 15 to 30 bp in length, wherein the target sequence is adjacent to the 5′-end or the 3′-end of a protospacer-adjacent motif (PAM) sequence recognized by Cas12f1 protein or a variant thereof which is present in a region 5000 bp, 4000 bp, 3000 bp, 2000 bp, or 1000 bp upstream of dystrophin exon 51 or a region 5000 bp, 4000 bp, 3000 bp, 2000 bp, or 1000 bp downstream of dystrophin exon 51.

In an embodiment, a target region comprising the target sequence may comprise a protospacer sequence selected from SEQ ID NOs: 190 to 311. The target sequence may be a sequence complementary to a protospacer sequence selected from SEQ ID NOs: 190 to 311 within the target region.

In an embodiment, the guide sequence of the guide RNA may bind complementarily to the target sequence. Complementary binding between the guide sequence and the target sequence may include at least one mismatch bond. For example, complementary binding between the guide sequence and the target sequence may include 0 to 5 mismatches. The guide sequence may be a sequence having at least 70% sequence complementarity to the target sequence. Unless otherwise stated, “complementary” may mean including 0 to 5 mismatches or having at least 70% complementarity, and should be interpreted appropriately depending on the context. When the target sequence is DNA, for an adenosine (A) present in the target sequence, the guide sequence may comprise a uridine (U) residue that can form a complementary bond with A.

In an embodiment, the target sequence may be a sequence of 15 to 40 nucleotides. For example, the target sequence may be a sequence of 15 to 20, 15 to 25, 15 to 30, 15 to 35, or 15 to 40 nucleotides. The target sequence may be a sequence of 20 to 25, 20 to 30, 20 to 35, or 20 to 40 nucleotides. In addition, the target sequence may be a sequence of 25 to 30, 25 to 35, or 25 to 40 nucleotides. In addition, the target sequence may be a sequence of 30 to 35 or 30 to 40 nucleotides. In addition, the target sequence may be a sequence of 35 to 40 nucleotides. In addition, the target sequence may be a sequence of 15, 16, 17, 18, 19, 20, 21, 22, 23, 24, 25, 26, 27, 28, 29, 30, 31, 32, 33, 34, 35, 36, 37, 38, 39, or 40 nucleotides.

In an embodiment, the guide sequence may be a sequence that is at least 70% to 75%, at least 70% to 80%, at least 70% to 85%, at least 70% to 90%, at least 70% to 95%, at least 70% to 100%, at least 75% to 80%, at least 75% to 85%, at least 75% to 90%, at least 75% to 95%, or at least 75% to 100% complementary to the target sequence. Specifically, the guide sequence may be a sequence that is at least 80% to 85%, at least 80% to 90%, at least 80% to 95%, at least 80% to 100%, at least 85% to 90%, at least 85% to 95%, or at least 85% to 100% complementary to the target sequence. More specifically, the guide sequence may be a sequence that is at least 90% to 95%, at least 90% to 100%, or at least 95% to 100% complementary to the target sequence. Even more specifically, the guide sequence may be a sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% complementary to the target sequence.

In another embodiment, the guide sequence may be a sequence identical with or similar to the protospacer sequence. The guide sequence may have at least 70% sequence identity to the protospacer sequence. For thymine (T) present in the protospacer sequence, the guide sequence may comprise uracil (U) instead of thymine (T).

In an embodiment, the guide sequence may have at least 70% to 75%, at least 70% to 80%, at least 70% to 85%, at least 70% to 90%, at least 70% to 95%, at least 70% to 100%, at least 75% to 80%, at least 75% to 85%, at least 75% to 90%, at least 75% to 95%, or at least 75% to 100% sequence identity or similarity to the protospacer sequence. Specifically, the guide sequence may have at least 80% to 85%, at least 80% to 90%, at least 80% to 95%, at least 80% to 100%, at least 85% to 90%, at least 85% to 95%, or at least 85% to 100% sequence identity or similarity to the protospacer sequence. More specifically, the guide sequence may have at least 90% to 95%, at least 90% to 100%, or at least 95% to 100% identity or similarity to the protospacer sequence. Even more specifically, the guide sequence may have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity or similarity to the protospacer sequence.

In an embodiment, the guide sequence may comprise or consist of a sequence hybridizable or complementary to a target sequence that is complementary to a nucleotide sequence selected from the group consisting of SEQ ID NOs: 190 to 311, wherein the nucleotide sequence is located in a non-target strand of a region 5000 bp upstream or downstream of dystrophin exon 51.

In an embodiment, the guide sequence may comprise or consist of a sequence hybridizable or complementary to a target sequence that is complementary to a nucleotide sequence selected from the group consisting of SEQ ID NOs: 190 to 217 and SEQ ID NOs: 255 to 280, wherein the nucleotide sequence is located in a non-target strand of a region 5000 bp upstream of dystrophin exon 51.

In an embodiment, the guide sequence may comprise or consist of a sequence hybridizable or complementary to a target sequence that is complementary to a nucleotide sequence selected from the group consisting of SEQ ID NOs: 218 to 254 and SEQ ID NOs: 281 to 311, wherein the nucleotide sequence is located in a non-target strand of a region 5000 bp downstream of dystrophin exon 51.

In an embodiment, the guide sequence may comprise or consist of a sequence of contiguous 15 to 20 nucleotides from a nucleotide sequence selected from the group consisting of SEQ ID NOs: 190 to 311, wherein thymine (T) in the contiguous nucleotide sequence is substituted with uracil (U). Specifically, the guide sequence may comprise or consist of a nucleotide sequence selected from the group consisting of SEQ ID NOs: 312 to 335.

In an embodiment, the guide sequence may comprise a sequence of contiguous 15 to 20 nucleotides from a nucleotide sequence selected from the group consisting of SEQ ID NOs: 190 to 217 and SEQ ID NOs: 255 to 280, wherein thymine (T) in the contiguous nucleotide sequence is substituted with uracil (U).

In an embodiment, the guide sequence may comprise a sequence of contiguous 15 to 20 nucleotides from a nucleotide sequence selected from the group consisting of SEQ ID NOs: 218 to 254 and SEQ ID NOs: 281 to 311, wherein thymine (T) in the contiguous nucleotide sequence is substituted with uracil (U).

In an embodiment, the guide sequence may comprise or consist of a nucleotide sequence selected from the group consisting of SEQ ID NOs: 312 to 323.

In an embodiment, the guide sequence may comprise or consist of a nucleotide sequence selected from the group consisting of SEQ ID NOs: 324 to 335.

In an embodiment, when two or more guide RNAs are used in the Cas12f1 system, the first guide sequence may be a sequence hybridizable to a target sequence of contiguous 15 to 30 bp in length, wherein the target sequence is adjacent to the 5′-end or the 3′-end of a PAM sequence present in a region 5000 bp upstream of dystrophin exon 51, and the second guide sequence may be a sequence hybridizable to a target sequence of contiguous 15 to 30 bp in length, wherein the target sequence is adjacent to the 5′-end or the 3′-end of a PAM sequence present in a region 5000 bp downstream of dystrophin exon 51.

In another embodiment, the first guide sequence may be a sequence hybridizable to a target sequence that is complementary to a nucleotide sequence selected from the group consisting of SEQ ID NOs: 190 to 217 and SEQ ID NOs: 255 to 280, wherein the nucleotide sequence is located in a non-target strand of a region 5000 bp upstream of dystrophin exon 51 and/or
the second guide sequence may be a sequence hybridizable to a target sequence that is complementary to a nucleotide sequence selected from the group consisting of SEQ ID NOs: 218 to 254 and SEQ ID NOs: 281 to 311, wherein the nucleotide sequence is located in a non-target strand of a region 5000 bp downstream of dystrophin exon 51.

In yet another embodiment, the first guide sequence may comprise a sequence of contiguous 15 to 20 nucleotides from a nucleotide sequence selected from the group consisting of SEQ ID NOs: 190 to 217 and SEQ ID NOs: 255 to 280, wherein thymine (T) in the contiguous nucleotide sequence is substituted with uracil (U), and/or the second guide sequence may comprise a sequence of contiguous 15 to 20 nucleotides from a nucleotide sequence selected from the group consisting of SEQ ID NOs: 218 to 254 and SEQ ID NOs: 281 to 311, wherein thymine (T) in the contiguous nucleotide sequence is substituted with uracil (U).

In still yet another embodiment, the first guide sequence may comprise or consist of a nucleotide sequence selected from the group consisting of SEQ ID NOs: 312 to 323, and/or the second guide sequence may comprise or consist of a nucleotide sequence selected from the group consisting of SEQ ID NOs: 324 to 335.

In an embodiment, the guide sequence may be present at the 3′-end of the crRNA. In another embodiment, a U-rich tail may be added to the 3′-end of the guide sequence. The U-rich tail is described below.

### 2.2. Engineered Guide RNA

Since no naturally occurring gRNA has been found for CWCas12f1 according to an embodiment of the present disclosure, it was intended to produce an optimal gRNA that exhibits highly efficient targeting and editing activity not only for the engineered UnCas12f1 protein but also for the engineered CWCas12f1 protein. From this perspective, the gRNA may be a wild-type gRNA found in nature for wild-type UnCas12f1, which is similar in size to the CWCas12f1 protein. That is, in the present disclosure, the “wild-type” gRNA for the engineered Cas12f1 protein was used to mean “basic” or “canonical” gRNA.

In an embodiment, the gRNA for the engineered Cas12f1 protein is characterized by being an engineered guide RNA in which a new configuration is added to a wild-type guide RNA found in nature, or an existing structure of the wild-type guide RNA is removed and/or replaced, or a structure of the wild-type guide RNA is partially modified.

In an embodiment, the engineered gRNA is an engineered gRNA comprising a sequence having the wild-type gRNA sequence in which at least one nucleotide has been substituted, deleted, inserted, or added, wherein the sequence excluding the guide sequence has at least 50%, 55%, 60%, 65%, 70%, 75%, 80%, 85%, 90%, 91%, 92%, 93%, 94%, 95%, 96%, 97%, 98%, 99%, or 100% sequence identity to the wild-type Cas12f1 gRNA. In the context of RNA, nucleic acid, or polypeptide, the term “sequence identity” refers to a value determined by comparing two sequences that are optimally aligned over a comparison window, in which a sequence portion of RNA, nucleic acid, and the like within the comparison window may comprise insertion or deletion (that is, gap) relative to the reference sequence to achieve optimal alignment.

Hereinafter, the structures of wild-type and engineered gRNAs and modifications thereof will be described in detail for each of the five modification sites. The modification site is abbreviated as “MS” throughout this specification, and the numbers following “modification site” or “MS” are sequentially assigned depending on engineering flow of each modification site according to an embodiment. However, this does not mean that engineering (modification) at a modification site with a later number necessarily includes engineering (modification) at a modification site with an earlier number. FIG. 2 illustrates modification sites MS1 to MS5, which are included in the engineered guide RNA according to an embodiment of the present disclosure, on the wild-type guide RNA sequence.

The modifications applied to the engineered guide RNA (gRNA) of the present disclosure are ultimately intended to achieve high gene editing efficiency while deriving a gRNA that is shorter in length. That is, the modifications disclosed in the present disclosure are intended to produce an engineered gRNA of a shorter length having equal or improved recognition/cleavage efficiency for a target nucleic acid compared to the wild-type gRNA of a longer length, thereby allowing more space to be allocated to other components (for example, additional guide RNAs and shRNAs for inhibiting specific gene expression) for various purposes or uses within the packaging limit (about 4.7 kb) of a delivery vehicle such as adeno-associated virus (AAV). This provides a highly efficient gene editing effect that could not be achieved with the existing CRISPR/Cas system.

Therefore, the engineered gRNA provided in the present disclosure basically comprises a sequence having the wild-type Cas12f1 gRNA sequence in which one or more nucleotides are substituted, deleted, inserted, or added. Here, for the engineered gRNA, a portion thereof excluding the guide sequence may have sequence identity of 50% or more, 60% or more, 70% or more, 80% or more, 85% or more, 90% or more, or 95% or more to the wild-type Cas12f1 gRNA.

In an embodiment, compared to the wild-type Cas12f1 gRNA comprising (i) at least one stem region, (ii) a tracrRNA-crRNA complementarity region, and optionally (iii) a region comprising three or more consecutive uracil (U) residues, the engineered gRNA of the present disclosure may comprise at least one modification selected from the group consisting of (a) deletion of at least a part of the at least one stem region; (b) deletion of at least a part of the tracrRNA-crRNA complementarity region; (c) replacement of one or more of uracil (U) residues when three or more consecutive uracil (U) residues are present; and (d) addition of one or more uridine residues to the 3′-end of the crRNA sequence.

In another embodiment, the engineered guide RNA may comprise at least one modification selected from the group consisting of (a1) deletion of at least a part of the first stem region; (a2) deletion of at least a part of the second stem region; (b) deletion of at least a part of the tracrRNA-crRNA complementarity region; (c) replacement of one or more U residues with A, G or C in three or more consecutive uracil (U) residues when the consecutive U residues are present in the tracrRNA-crRNA complementarity region; and (d1) addition of a U-rich tail to the 3′-end of the crRNA sequence (in which a sequence of the U-rich tail is represented by 5′-(UₘV)ₙUₒ-3′, wherein V is each independently A, C, or G, m and o are integers between 1 and 20, and n is an integer between 0 and 5).

In another embodiment, the engineered guide RNA may be represented by the following Formula (I).

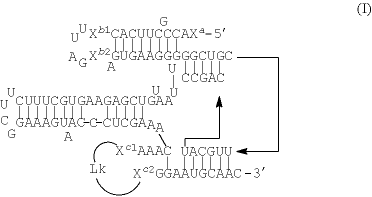

In Formula (I),
X^{a}, X^{b1}, X^{b2}, X^{c1}, and X^{c2} each independently consist of 0 to 35 (poly)nucleotides,
X^{g} is a guide sequence,
Lk is a polynucleotide linker of 2 to 20 nucleotides in length or is absent, and
(UₘV)ₙUₒ is a U-rich tail and is present or absent, and when (UₘV)ₙUₒ is present, U is uridine, V is each independently A, C, or G, m and o are each independently an integer between 1 and 20, and n is an integer between 0 and 5.

[In Formula (I), the black solid line represents a chemical bond (for example, a phosphodiester bond) between nucleotides or specific molecules, and the gray thick line represents a complementary bond between nucleotides].

In an embodiment, X^{a} may be absent or a (poly)nucleotide having a stem-loop conformation.

In an embodiment, X^{b1} and X^{b2} may be (poly)nucleotides capable of complementary binding to each other.

In an embodiment, X^{c1} and X^{c2} may be (poly)nucleotides capable of complementary binding to each other.

In yet another embodiment, the engineered guide RNA may have at least 70%, at least 80%, at least 85%, at least 90%, at least 95%, or at least 98% sequence identity to the sequence represented by Formula (I). Here, the sequence identity with Formula (I) is based on the sequence excluding the regions indicated by the symbols.

When referring to the scaffold region of the wild-type guide RNA, the first stem region of the scaffold sequence may be a region corresponding to X^{a} in Formula (I). The second stem region of the scaffold sequence may be a region corresponding to X^{b1} and X^{b2} in Formula (I). The third stem region of the scaffold sequence may be a region corresponding to the sequence 5′-GGCUGCUUGCAUCAGCC-3′ in Formula (I). The fourth stem region of the scaffold sequence may be a region corresponding to the sequence 5′-UCGAGAAGUGCUUUCUUCGGAAAGUAACCCUCGA-3′ in Formula (I). In addition, the tracrRNA-crRNA complementarity region (the fifth stem region) of the scaffold sequence may be a region corresponding to X^{c1} and X^{c2} in Formula (I).

Hereinafter, modifications at respective modification sites in the engineered gRNA will be described in detail.

### (1) Modification at Modification Site 1 (MS1)

This section describes a modification at MS1. In an embodiment, wild-type tracrRNA (for example, SEQ ID NO: 11), which may be a guide RNA (gRNA) existing in nature, may have a sequence containing five consecutive uracil (U) residues therein. This poses a problem in that, in a case of attempting to express the wild-type tracrRNA in a cell using a vector or the like, such a sequence acts as a transcription termination signal under certain conditions, thereby causing unintended early termination of transcription. That is, in a case where the sequence containing five consecutive U residues acts as a transcription termination signal, normal or complete expression of the tracrRNA is inhibited, and formation of normal or complete gRNA is also inhibited, which consequently decreases efficiency of cleavage or homology-directed repair of the target nucleic acid editing system of the present disclosure for the target nucleic acid or target gene.

Therefore, in order to solve the above-mentioned problem, the engineered gRNA may be such that at least one uracil (U) of three or more, four or more, or five or more consecutive U residues, preferably four or five U residues, which are contained in the wild-type tracrRNA (for example, SEQ ID NO: 11), is artificially modified into another nucleotide such as A, C, T, or G.

In an embodiment, the engineered gRNA is provided which comprises a modification, in which at least one of three or more consecutive U residues is substituted with a different type of nucleotide, in a region containing three or more consecutive U residues, referred to as MS1. For example, the three or more consecutive U residues may be present in the tracrRNA-crRNA complementarity region of the tracrRNA, wherein a modification may be made by substituting at least one of the three or more U residues with A, G, or C such that no sequence with three or more consecutive U residues exists.

Here, it is preferable that the sequence within the tracrRNA-crRNA complementarity region of crRNA, which corresponds to the sequence to be modified, is also modified together. In an embodiment, when there is the sequence 5′-ACGAA-3′ within the tracrRNA-crRNA complementarity region of crRNA, which forms a partial complementary bond with the sequence 5′-UUUUU-3′ within the tracrRNA-crRNA complementarity region of tracrRNA, this sequence may be replaced with 5′-NGNNN-3′. Here, N is each independently A, C, G, or U.

In another embodiment, MS1 may be present in the polynucleotides indicated by X^{c1} and X^{c2} in Formula (I).

In an embodiment, the engineered gRNA of Formula (I) may comprise a modification in which one or more of the U residues are substituted with A, G, or C, when three or more consecutive uracil (U) residues are present in the X^{c1} sequence. For example, when the sequence 5′-UUUUU-3′ is present in the X^{c1} sequence, the sequence may be replaced with 5′-NNNCN-3′, wherein N is each independently A, C, G, or U. As a more specific example, the sequence 5′-UUUUU-3′ in the X^{c1} sequence may be replaced by any one nucleotide sequence selected from the group consisting of the following sequences; however, the replacing sequence is not limited to the following sequences as long as it prevents appearance of a sequence containing three or more consecutive U residues: 5′-UUUCU-3′, 5′-GUUCU-3′, 5′-UCUCU-3′, 5′-UUGCU-3′, 5′-UUUCC-3′, 5′-GCUCU-3′, 5′-GUUCC-3′, 5′-UCGCU-3′, 5′-UCUCC-3′, 5′-UUGCC-3′, 5′-GCGCU-3′, 5′-GCUCC-3′, 5′-GUGCC-3′, 5′-UCGCC-3′, 5′-GCGCC-3′, and 5′-GUGCU-3′.

In another embodiment, in the engineered gRNA of Formula (I), the X^{c2} sequence comprises a region in which at least a part of the sequence forms a complementary bond with the X^{c1} sequence (also referred to as a tracrRNA-crRNA complementarity region), wherein a corresponding sequence in the X^{c2} sequence, which forms at least one complementary bond with 3 or more consecutive U residues present in the X^{c1} sequence, may also be modified. For example, when the sequence 5′-ACGAA-3′ is present in the X^{c2} sequence, the sequence may be replaced with 5′-NGNNN-3′, wherein N is each independently A, C, G, or U. As a more specific example, the sequence 5′-ACGAA-3′ in the X^{c1} sequence may be replaced by any one nucleotide sequence selected from the group consisting of the following sequences; however, the replacing sequence is not limited to the following sequences: 5′-AGGAA-3′, 5′-AGCAA-3′, 5′-AGAAA-3′, 5′-AGCAU-3′, 5′-AGCAG-3′, 5′-AGCAC-3′, 5′-AGCUA-3′, 5′-AGCGA-3′, 5′-AGCCA-3′, 5′-UGCAA-3′, 5′-UGCUA-3′, 5′-UGCGA-3′, 5′-UGCCA-3′, 5′-GGCAA-3′, 5′-GGCUA-3′, 5′-GGCGA-3′, 5′-GGCCA-3′, 5′-CGCAA-3′, 5′-CGCUA-3′, 5′-CGCGA-3′, and 5′-CGCCA-3′.

In another embodiment, when a sequence containing 3 or more consecutive U residues in the X^{c1} sequence is modified into another sequence, it is preferred that the corresponding nucleotides in the X^{c2} sequence (that is, at least some of which form complementary bonds therewith) are modified so that they can form complementary bonds with the modified nucleotides. For example, when the sequence 5′-UUUUU-3′ in the X^{c1} sequence is modified into 5′-GUGCU-3′, it is preferred that the sequence 5′-ACGAA-3′ in the X^{c2} sequence is modified into 5′-AGCAA-3′; however, complementary bonding is not necessarily required.

### (2) Modification at Modification Site 2 (MS2)

This section describes a modification at MS2. In an embodiment, the engineered guide RNA (gRNA) may be obtained by adding a new configuration to the gRNA found in nature, and may be such that one or more uridine residues are added to the 3′-end of the crRNA sequence. Here, the 3′-end of the crRNA sequence may be the 3′-end of the guide sequence (spacer). In the present disclosure, the one or more uridine residues added to the 3′-end are also referred to herein as a U-rich tail. The engineered gRNA comprising one or more uridine residues or a U-rich tail added to the 3′-end serves to increase nucleic acid cleavage or indel efficiency of the hypercompact CRISPR/Cas12 system for a target gene or target nucleic acid.

The term “U-rich tail” as used herein may refer not only to an RNA sequence itself that is rich in uridine (U), but also a DNA sequence encoding the same, and this may be appropriately interpreted depending on the context. The present inventors have experimentally elucidated the structure and effects of the U-rich tail sequence in detail. The U-rich tail sequence will be described in more detail below with specific embodiments.

In an embodiment, the U-rich tail sequence may be represented by Ux, wherein x may be 1, 2, 3, 4, 5, 6, 7, 8, 9, 10, 11, 12, 13, 14, 15, 16, 17, 18, 19, or 20. For example, x may be an integer within a range of two numerical values selected from the numerical values listed above. For example, x may be an integer between 1 and 6. As another example, x may be an integer between 1 and 20. In an embodiment, x may be an integer of 20 or higher.

In another embodiment, the U-rich tail sequence is represented by 5′-(UₘV)ₙUₒ-3′, wherein V may be each independently A, C or G, m and o may be integers between 1 and 20, and n may be an integer between 0 and 5. As an example, n may be 0, 1, or 2. As an example, m and o may be each independently 1, 2, 3, 4, 5, 6, 7, 8, 9, or 10.

In another embodiment, the engineered guide RNA may be gRNA consisting of a sequence represented by Formula (I) or having at least 80%, at least 85%, at least 90%, or at least 95% sequence identity thereto. Here, MS2 is a region corresponding to (UₘV)ₙUₒ in Formula (I), wherein U is uridine, and V, m, o and n are as defined above.

Preferably, in the engineered gRNA represented by Formula (I), (UₘV)ₙUₒ may be a U-rich tail in which (i) n is 0 and o is an integer between 1 and 6, or (ii) V is A or G, m and o are each independently an integer between 3 and 6, and n is an integer between 1 and 3. In a specific example, (UₘV)ₙUₒ in Formula (I) may be a U rich tail consisting of any one sequence selected from the group consisting of 5′-U-3′, 5′-UU-3′, 5′-UUU-3′, 5′-UUUU-3′, 5′-UUUUU-3′, 5′-UUUUUU-3′, 5′-UUURUUU-3′, 5′-UUURUUURUUU-3′, 5′-UUUURU-3′, 5′-UUUURUU-3′, 5′-UUUURUUU-3′, 5′-UUUURUUUU-3′, 5′-UUUURUUUUU-3′, and 5′-UUUURUUUUUU-3′, wherein R is A or G.

In yet another embodiment, the U-rich tail sequence may comprise a modified uridine repeat sequence that contains a non-uridine ribonucleoside (A, C, or G) for every 1 to 5 repetitions of uridine. The modified uridine repeat sequence is particularly useful in a case of designing a vector that expresses an engineered crRNA. In an embodiment, the U-rich tail sequence may comprise a sequence in which UV, UUV, UUUV, UUUUV, and/or UUUUUV are repeated one or more times. Here, V is one of A, C or G.

In addition, the U-rich tail sequence may be a combination of the sequence represented by Ux and the sequence represented by (UaV)n. In an embodiment, the U-rich tail sequence may be represented by (U)n1-V1-(U)n2-V2-Ux. Here, V1 and V2 are each one of adenine (A), cytidine (C), and guanine (G). Here, n1 and n2 may each be an integer between 1 and 4. Here, x may be an integer between 1 and 20. In addition, the U-rich tail sequence may have a length of 1 nt, 2 nts, 3 nts, 4 nts, 5 nts, 6 nts, 7 nts, 8 nts, 9 nts, 10 nts, 11 nts, 12 nts, 13 nts, 14 nts, 15 nts, 16 nts, 17 nts, 18 nts, 19 nts, or 20 nts. In an embodiment, the U-rich tail sequence may have a length of 20 nts or longer.

In another embodiment, when the engineered gRNA is expressed in a cell, the U-rich tail may exist in a plurality of forms due to premature termination of transcription. For example, according to an embodiment, when a gRNA intended to contain a U-rich tail of the sequence 5′-UUUUAUUUUUU-3′ is transcribed in a cell, four or more or five or more T residues may act as a termination sequence, and thus gRNAs containing a U-rich tail such as 5′-UUUUAUUUU-3′, 5′-UUUUAUUUUU-3′, or 5′-UUUUAUUUUUU-3′ may be produced simultaneously. Therefore, in the present disclosure, a U-rich tail containing four or more U residues may be understood to also include a U-rich tail sequence having a shorter length than the intended length.

In yet another embodiment, the U-rich tail sequence may comprise additional nucleotides other than uridine, depending on the environment where the CRISPR/Cas12 system is actually used and expression environment, such as the internal environment of a eukaryotic cell or a prokaryotic cell.

### (3) Modification at Modification Site 3 (MS3)

This section describes a modification at MS3. As described above, MS3 refers to a region (which may be referred to as the first stem region) that comprises at least a part of the nucleotides forming a stem structure within a complex of the gRNA with an effector protein. The MS3 may comprise a region that does not interact with the effector protein when the gRNA and effector protein form a complex. The modification at MS3 involves removal of at least a part of the first stem region near the 5′-end of tracrRNA.

In an embodiment, the engineered gRNA comprises a modification in which at least a part of the first stem region (for example, the sequence of SEQ ID NO: 14) is deleted.

In another embodiment, the engineered gRNA comprises a modification in which at least a part of the first stem region on tracrRNA is deleted, wherein the at least a part of the first stem region to be deleted may consist of 1 to 20 nucleotides. Specifically, at least a part of the first stem region may consist of 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, 19, or 20 nucleotides.

In yet another embodiment, the MS3 or the first stem region is a portion corresponding to the polynucleotide indicated by X^{a} in Formula (I), wherein due to a modification in which at least a part of the first stem region is deleted, X^{a} may consist of 0 to 35 (poly)nucleotides, preferably 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 1, or 0 (poly)nucleotides.

In an embodiment, in the engineered gRNA of Formula (I), X^{a} may comprise the nucleotide sequence of SEQ ID NO: 14 or may comprise a nucleotide sequence having at least a part thereof, preferably a deleted form of the sequence of SEQ ID NO: 14 with 1 to 20 nucleotides deleted therefrom. For example, the nucleotide deletion may involve random deletion of at least 1, 2, 3, 5, 6, 7, 8, 9, 10, 11, 12, 13, 14 15, 16, 17, 18, 19, or 20 nucleotides from the sequence of SEQ ID NO: 14. As a preferred example, the nucleotide deletion may involve sequential deletion of at least 1, 2, 3, 5, 6, 7, 8, 9, 10, 11, 12, 13, 14 15, 16, 17, 18, 19, or 20 nucleotides from the 5′-end of the sequence of SEQ ID NO: 14. More specifically, X^{a} may comprise or consist of 5′-CUUCACUGAUAAAGUGGAGA-3′ (SEQ ID NO: 14), 5′-UUCACUGAUAAAGUGGAGA-3′ (SEQ ID NO: 15), 5′-UCACUGAUAAAGUGGAGA-3′ (SEQ ID NO: 16), 5′-CACUGAUAAAGUGGAGA-3′ (SEQ ID NO: 17), 5′-ACUGAUAAAGUGGAGA-3′ (SEQ ID NO: 18), 5′-CUGAUAAAGUGGAGA-3′ (SEQ ID NO: 19), 5′-UGAUAAAGUGGAGA-3′ (SEQ ID NO: 20), 5′-GAUAAAGUGGAGA-3′ (SEQ ID NO: 21), 5′-AUAAAGUGGAGA-3′ (SEQ ID NO: 22), 5′-UAAAGUGGAGA-3′ (SEQ ID NO: 23), 5′-AAAGUGGAGA-3′ (SEQ ID NO: 24), 5′-AAGUGGAGA-3′, 5′-AGUGGAGA-3′, 5′-GUGGAGA-3′, 5′-UGGAGA-3′, 5′-GGAGA-3′, 5′-GAGA-3′, 5′-AGA-3′, 5′-GA-3′, or 5′-A-3′, or X^{a} may be absent.

### (4) Modification at Modification Site 4 (MS4)

This section describes a modification at MS4. MS4 refers to a region spanning the 3′-end of tracrRNA and the 5′-end of crRNA, or, in a case of a single guide RNA form, a region where the sequence corresponding to tracrRNA and the sequence corresponding to crRNA form at least partial complementary bonding. MS4 may comprise at least a part of the sequence referred to as the tracrRNA-crRNA complementarity region (which may also be referred to as the fifth stem region). In the present disclosure, the tracrRNA-crRNA complementarity region may comprise both modification site 1 (MS1) and modification site 4 (MS4). The modification at MS4 comprises deletion of at least a part of the tracrRNA-crRNA complementarity region. The tracrRNA-crRNA complementarity region may comprise a part of tracrRNA and a part of crRNA. In this regard, the tracrRNA-crRNA complementarity region may comprise nucleotides such that partial nucleotides contained in tracrRNA can form complementary bonds with partial nucleotides contained in crRNA within a complex of gRNA with the nucleic acid degrading protein, and may comprise nucleotides adjacent thereto. The tracrRNA-crRNA complementarity region of tracrRNA may comprise a region that does not interact with the nucleic acid degrading protein within a complex of gRNA with the nucleic acid degrading protein.

In some embodiments, the engineered gRNA comprises deletion of at least a part of the tracrRNA-crRNA complementarity region in tracrRNA, deletion of at least a part of the tracrRNA-crRNA complementarity region in crRNA, or deletion of at least a part of the tracrRNA-crRNA complementarity region in both the tracrRNA and the crRNA.

In another embodiment, the engineered gRNA comprises a modification in which a part of the tracrRNA-crRNA complementarity region is deleted, wherein the part of the complementarity region to be deleted may consist of 1 to 54 nucleotides.

In yet another embodiment, the engineered gRNA comprises a modification in which the entire tracrRNA-crRNA complementarity region is deleted, wherein the entire complementarity region to be deleted may consist of 55 nucleotides.

In an embodiment, the tracrRNA-crRNA complementarity region may comprise the nucleotide sequence of SEQ ID NO: 39 and/or the nucleotide sequence of SEQ ID NO: 58.

In another embodiment, the tracrRNA-crRNA complementarity region may further comprise a linker sequence.

Specifically, at least a part of the tracrRNA-crRNA complementarity region may consist of 3 to 55, 5 to 55, 7 to 55, 9 to 55, 11 to 55, 13 to 55, 15 to 55, 17 to 55, 19 to 55, 21 to 55, 23 to 55, 25 to 55, 27 to 55, 29 to 55, 31 to 55, 33 to 55, 35 to 55, 37 to 55, 39 to 55, or 41 to 55 nucleotides, preferably 42 to 55, 43 to 55, 44 to 55, 45 to 55, 46 to 55, 47 to 55, 48 to 55, 49 to 55, 50 to 55, 51 to 55, 52 to 55, 53 to 55, or 54, or 55 nucleotides.

In yet another embodiment, MS4 or the tracrRNA-crRNA complementarity region is a region corresponding to the polynucleotide indicated by X^{c1} and X^{c2} in Formula (I), in which due to the modification where at least a part of the tracrRNA-crRNA complementarity region is deleted, X^{c1} and X^{c2} may each independently consist of 0 to 35 (poly)nucleotides.

Preferably, X^{c1} may consist of 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 1, or 0 (poly)nucleotides. In addition, preferably, X^{c2} may consist of 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 1, or 0 (poly)nucleotides.

In an embodiment, in the engineered gRNA of Formula (I), X^{c1} may comprise the nucleotide sequence of SEQ ID NO: 39 or a deleted form of the sequence of SEQ ID NO: 39 with 1 to 28 nucleotides deleted therefrom. Preferably, the nucleotide deletion may involve sequential removal of at least 1, 2, 3, 5, 6, 7, 8, 9, 10, 11, 12, 13, 14, 15, 16, 17, 18, 19, 20, 21, 22, 23, 24, 25, 26, 27, or 28 nucleotides from the 5′-end of the sequence of SEQ ID NO: 39. More specifically, X^{c1} may comprise or consist of 5′-UUCAUUUUUCCUCUCCAAUUCUGCACAA-3′ (SEQ ID NO: 39), 5′-UUCAUUUUUCCUCUCCAAUUCUGCACA-3′ (SEQ ID NO: 40), 5′-UUCAUUUUUCCUCUCCAAUUCUGCAC-3′ (SEQ ID NO: 41), 5′-UUCAUUUUUCCUCUCCAAUUCUGCA-3′ (SEQ ID NO: 42), 5′-UUCAUUUUUCCUCUCCAAUUCUGC-3′ (SEQ ID NO: 43), 5′-UUCAUUUUUCCUCUCCAAUUCUG-3′ (SEQ ID NO: 44), 5′-UUCAUUUUUCCUCUCCAAUUCU-3′ (SEQ ID NO: 45), 5′-UUCAUUUUUCCUCUCCAAUUC-3′ (SEQ ID NO: 46), 5′-UUCAUUUUUCCUCUCCAAUU-3′ (SEQ ID NO: 47), 5′-UUCAUUUUUCCUCUCCAAU-3′ (SEQ ID NO: 48), 5′-UUCAUUUUUCCUCUCCAA-3′ (SEQ ID NO: 49), 5′-UUCAUUUUUCCUCUCCA-3′ (SEQ ID NO: 50), 5′-UUCAUUUUUCCUCUCC-3′ (SEQ ID NO: 51), 5′-UUCAUUUUUCCUCUC-3′ (SEQ ID NO: 52), 5′-UUCAUUUUUCCUCU-3′ (SEQ ID NO: 53), 5′-UUCAUUUUUCCUC-3′ (SEQ ID NO: 54), 5′-UUCAUUUUUCCU-3′ (SEQ ID NO: 55), 5′-UUCAUUUUUCC-3′ (SEQ ID NO: 56), 5′-UUCAUUUUUC-3′ (SEQ ID NO: 57), 5′-UUCAUUUUU-3′, 5′-UUCAUUUU-3′, 5′-UUCAUUU-3′, 5′-UUCAUU-3′, 5′-UUCAU-3′, 5′-UUCA-3′, 5′-UUC-3′, 5′-UU-3′, or 5′-U-3′, or X^{c1} may be absent.

Here, in a case where there is a region containing 3, 4, or 5 or more uracil (U) residues in the sequence of X^{c1} from which some nucleotides have been removed, the modification at MS1 as described above may also apply. For details about MS1, see the section “(1) Modification at modification site 1 (MS1).”

In another embodiment, in the engineered gRNA of Formula (I), X^{c2} may comprise the nucleotide sequence of SEQ ID NO: 58 or a deleted form of the sequence of SEQ ID NO: 58 with 1 to 27 nucleotides deleted therefrom. Preferably, the nucleotide deletion may involve sequential removal of at least 1, 2, 3, 5, 6, 7, 8, 9, 10, 11, 12, 13, 14, 15, 16, 17, 18, 19, 20, 21, 22, 23, 24, 25, 26, or 27 nucleotides from the 5′-end of the sequence of SEQ ID NO: 58. More specifically, X^{c2} may comprise or consist of 5′-GUUGCAGAACCCGAAUAGACGAAUGAA-3′ (SEQ ID NO: 58), 5′-UUGCAGAACCCGAAUAGACGAAUGAA-3′ (SEQ ID NO: 59), 5′-UGCAGAACCCGAAUAGACGAAUGAA-3′ (SEQ ID NO: 60), 5′-GCAGAACCCGAAUAGACGAAUGAA-3′ (SEQ ID NO: 61), 5′-CAGAACCCGAAUAGACGAAUGAA-3′ (SEQ ID NO: 62), 5′-AGAACCCGAAUAGACGAAUGAA-3′ (SEQ ID NO: 63), 5′-GAACCCGAAUAGACGAAUGAA-3′ (SEQ ID NO: 64), 5′-AACCCGAAUAGACGAAUGAA-3′ (SEQ ID NO: 65), 5′-ACCCGAAUAGACGAAUGAA-3′ (SEQ ID NO: 66), 5′-CCCGAAUAGACGAAUGAA-3′ (SEQ ID NO: 67), 5′-CCGAAUAGACGAAUGAA-3′ (SEQ ID NO: 68), 5′-CGAAUAGACGAAUGAA-3′ (SEQ ID NO: 69), 5′-GAAUAGACGAAUGAA-3′ (SEQ ID NO: 70), 5′-AAUAGACGAAUGAA-3′ (SEQ ID NO: 71), 5′-AUAGACGAAUGAA-3′ (SEQ ID NO: 72), 5′-UAGACGAAUGAA-3′ (SEQ ID NO: 73), 5′-AGACGAAUGAA-3′ (SEQ ID NO: 74), 5′-GACGAAUGAA-3′ (SEQ ID NO: 75), 5′-ACGAAUGAA-3′, 5′-CGAAUGAA-3′, 5′-GAAUGAA-3′, 5′-AAUGAA-3′, 5′-AUGAA-3′, 5′-UGAA-3′, 5′-GAA-3′, 5′-AA-3′, or 5′-A-3′, or X^{c2} may be absent.

Here, in a case where there is a sequence corresponding a sequence containing 3 or more, or 3, 4, or 5 or more uracil (U) residues in the sequence of X^{c2} from which some nucleotides have been removed, the modification at MS1 as described above may also apply. For details regarding MS1, see the section “(1) Modification at modification site 1 (MS1).”

In the engineered gRNA of Formula (I), the regions corresponding to X^{c1} and X^{c2} may each independently undergo the above-described modification. However, MS4 or the tracrRNA-crRNA complementarity region is a region where tracrRNA and crRNA form complementary bonds. For the tracrRNA and the crRNA to function as a dual guide RNA, it is preferable that the position and number of nucleotides to be deleted in each of X^{c1} and X^{c2} be identical with or similar to each other. That is, in order to preserve complementarity, in a case of sequentially deleting nucleotides from the 3′-end of tracrRNA in MS4 (tracrRNA-crRNA complementarity region), it is preferable to sequentially delete nucleotides from the 5′-end of crRNA.

In some embodiments, the 3′-end of X^{c1} and the 5′-end of X^{c2} in the engineered gRNA of Formula (I) may be linked by a linker (Lk) so that the gRNA is modified into a single guide RNA (sgRNA) form. The linker Lk is a sequence that physically or chemically connects tracrRNA and crRNA, and may be a polynucleotide sequence having a length of 1 to 30 nucleotides. In an embodiment, Lk may be a sequence of 1 to 5, 5 to 10, 10 to 15, 2 to 20, 15 to 20, 20 to 25, or 25 to 30 nucleotides. For example, Lk may be, but is not limited to, 5′-GAAA-3′. As another example, Lk may be a linker comprising or consisting of 5′-UUAG-3′, 5′-UGAAAA-3′, 5′-UUGAAAAA-3′, 5′-UUCGAAAGAA-3′ (SEQ ID NO: 76), 5′-UUCAGAAAUGAA-3′ (SEQ ID NO: 77), 5′-UUCAUGAAAAUGAA-3′ (SEQ ID NO: 78), or 5′-UUCAUUGAAAAAUGAA-3′ (SEQ ID NO: 79).

Meanwhile, while it is possible to use a linker (Lk) to make a single guide RNA (sgRNA), it is also possible to directly connect the 3′-end of tracrRNA, of which a partial sequence has been removed, to the 5′-end of crRNA of which a partial sequence has been removed.

In another embodiment, a case where X^{c1} and X^{c2} in the engineered gRNA of Formula (I) are linked by a linker may be indicated by 5′-X^{c1}-Lk-X^{c2}-3′ as in Formula (I), and the 5′-X^{c1}-Lk-X^{c2}-3′ may be any one nucleotide sequence selected from the group consisting of SEQ ID NOs: 80 to 86, but is not limited thereto.

### (5) Modification at Modification Site 5 (MS5)

This section describes a modification at MS5. As described above, MS5 corresponds to a region located toward the 3′-end of tracrRNA, which is referred to as the second stem region. The second stem region may comprise nucleotides that form a stem structure within a complex of the guide RNA (gRNA) with nucleic acid editing protein, and may comprise nucleotides adjacent thereto. Here, the stem structure is distinct from the stem included in the above-described first stem region.

In an embodiment, the engineered gRNA comprises a modification in which at least a part of the second stem region is deleted.

In another embodiment, the engineered gRNA comprises deletion of at least a part of the second stem region, wherein the at least a part of the second stem region to be deleted may consist of 1 to 27 nucleotides. Specifically, the at least a part of the second stem region may consist of 2 to 27, 3 to 27, 4 to 27, 5 to 27, 6 to 27, 7 to 27, 8 to 27, 9 to 27, 10 to 27, 11 to 27, 12 to 27, 13 to 27, 14 to 27, 15 to 27, 16 to 27, 17 to 27, 18 to 27, 19 to 27, 20 to 27, 21 to 27, 22 to 27, 23 to 27, 24 to 27, 25 to 27, 26, or 27 nucleotides.

In an embodiment, the second stem region may comprise or consist of the nucleotide sequence of SEQ ID NO: 25 and/or the nucleotide sequence of SEQ ID NO: 29.

In another embodiment, MS5 or the second stem region is a region comprising a (poly)nucleotide (comprising a loop of 5′-UUAG-3′) that is adjacent to the polynucleotide indicated by X^{b1} and X^{b2} in Formula (I), in which due to the modification where at least the part of the second stem region is deleted, X^{b1} and X^{b2} may each independently consist of 0 to 35 (poly)nucleotides.

Preferably, X^{b1} in Formula (I) may consist of 0 to 13, 0 to 12, 0 to 11, 0 to 10, 0 to 9, 0 to 8, 0 to 7, 0 to 6, 0 to 5, 0 to 4, 0 to 3, 0 to 2, 1, or 0 (poly)nucleotides. In addition, preferably, X^{b2} may consist of 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 1, or 0 (poly)nucleotides.

In an embodiment, in the engineered gRNA of Formula (I), X^{b1} may comprise the nucleotide sequence of SEQ ID NO: 25 or a deleted form of the sequence of SEQ ID NO: 25 with 1 to 13 nucleotides deleted therefrom. Preferably, the nucleotide deletion may involve sequential removal of at least 1, 2, 3, 5, 6, 7, 8, 9, 10, 11, 12, or 13 nucleotides from the 5′-end of the sequence of SEQ ID NO: 25. More specifically, X^{b1} may comprise or consist of 5′-CAAAAGCUGUCCC-3′ (SEQ ID NO: 25), 5′-CAAAAGCUGUCC-3′ (SEQ ID NO: 26), 5′-CAAAAGCUGUC-3′ (SEQ ID NO: 27), 5′-CAAAAGCUGU-3′ (SEQ ID NO: 28), 5′-CAAAAGCUG-3′, 5′-CAAAAGCU-3′, 5′-CAAAAGC-3′, 5′-CAAAAG-3′, 5′-CAAAA-3′, 5′-CAAA-3′, 5′-CAA-3′, 5′-CA-3′, or 5′-C-3′, or X^{b1} may be absent.

In another embodiment, in the engineered gRNA of Formula (I), X^{b2} may comprise the nucleotide sequence of SEQ ID NO: 29 or a deleted form of the sequence of SEQ ID NO: 29 with 1 to 14 nucleotides deleted therefrom. Preferably, the nucleotide deletion may involve sequential removal of at least 1, 2, 3, 5, 6, 7, 8, 9, 10, 11, 12, 13, or 14 nucleotides from the 5′-end of the sequence of SEQ ID NO: 29. More specifically, X^{b2} may comprise or consist of 5′-GGGAUUAGAACUUG-3′ (SEQ ID NO: 29), 5′-GGAUUAGAACUUG-3′ (SEQ ID NO: 30), 5′-GAUUAGAACUUG-3′ (SEQ ID NO: 31), 5′-AUUAGAACUUG-3′ (SEQ ID NO: 32), 5′-UUAGAACUUG-3′ (SEQ ID NO: 33), 5′-UAGAACUUG-3′, 5′-AGAACUUG-3′, 5′-GAACUUG-3′, 5′-AACUUG-3′, 5′-ACUUG-3′, 5′-CUUG-3′, 5′-UUG-3′, 5′-UG-3′, or 5′-G-3′, or X^{b2} may be absent.

In the engineered gRNA of Formula (I), the regions corresponding to X^{b1} and X^{b2} may be each independently modified. However, for normal preservation of the stem-loop structure, it is preferable that the position and number of nucleotides to be deleted in each of X^{b1} and X^{b2} be identical with or similar to each other. For example, in a case of sequentially deleting nucleotides from the 5′-end direction in X^{b1}, it is preferable to sequentially delete nucleotides from the 3′-end direction in X^{b2}

In another embodiment, a sequence of the loop portion connecting X^{b1} and X^{b2} in the engineered gRNA of Formula (I) is indicated by 5′-UUAG-3′, and this may be replaced with another sequence such as 5′-NNNN-3′ and 5′-NNN-3′, if necessary. Here, N is each independently A, C, G, or U. For example, the 5′-NNNN-3′ may be 5′-GAAA-3′, and the 5′-NNN-3′ may be 5′-CGA-3′.

For example, in the engineered gRNA of Formula (I), a sequence of the loop portion connecting X^{b1} and X^{b2} is 5′-UUAG-3′, and the sequence 5′-X^{b1}UUAGX^{b2}-3′ in Formula (I) may comprise or consist of any one nucleotide sequence selected from the group consisting of SEQ ID NOs: 34 to 38.

(6) Examples of gRNAs to which Modifications at Modification Sites 1 to 5 have been Applied

The engineered guide RNA (gRNA) included in the target nucleic acid editing system of the present disclosure may comprise modifications at two or more of the above-mentioned modification sites 1 (MS1) to 5 (MS5).

In some embodiments, the engineered guide RNA may comprise one or more modifications selected from the group consisting of (a1) deletion of at least a part of the first stem region; (a2) deletion at least a part of the second stem region; (b) deletion of at least a part of the tracrRNA-crRNA complementarity region; (c) replacement of one or more uracil (U) residues with A, G, or C in three or more consecutive U residues when the consecutive U residues are present in the tracrRNA-crRNA complementarity region; and (d1) addition of a U-rich tail to the 3′-end of the crRNA sequence. The U-rich tail sequence may be represented by 5′-(UₘV)ₙUₒ-3′, wherein V is each independently A, C, or G, m and o are integers between 1 and 20, and n is an integer between 0 and 5.

For example, the engineered guide RNA may comprise (d1) addition of a U-rich tail to the 3′-end of the crRNA sequence and (c) replacement of one or more uracil (U) residues with A, G, or C in three or more consecutive U residues when the consecutive U residues are present in the tracrRNA-crRNA complementarity region.

As another example, the engineered guide RNA may comprise (d1) addition of a U-rich tail to the 3′-end of the crRNA sequence, (c) replacement of one or more U residues with A, G or C in three or more consecutive U residues when the consecutive U residues are present in the tracrRNA-crRNA complementarity region, and (a1) deletion of at least a part of the first stem region.

As yet another example, the engineered guide RNA may comprise (d1) addition of a U-rich tail to the 3′-end of the crRNA sequence, (c) replacement of one or more U residues with A, G or C in three or more consecutive U residues when the consecutive U residues are present in the tracrRNA-crRNA complementarity region, and (a1) deletion of at least a part of the first stem region.

As still yet another example, the engineered guide RNA may comprise (d1) addition of a U-rich tail to the 3′-end of the crRNA sequence, (a1) deletion of at least a part of the first stem region, and (b) deletion of at least a part of the tracrRNA-crRNA complementarity region, wherein the engineered guide RNA may further comprise replacement of one or more U residues with A, G or C in three or more consecutive U residues when the consecutive U residues are present in the tracrRNA-crRNA complementarity region containing partial deletion.

As still yet another example, the engineered guide RNA may comprise (d1) addition of a U-rich tail to the 3′-end of the crRNA sequence, (a1) deletion of at least a part of the first stem region, (b) deletion of at least a part of the tracrRNA-crRNA complementarity region, and (a2) deletion of at least a part of the second stem region, wherein the engineered guide RNA may further comprise replacement of one or more U residues with A, G or C in three or more consecutive U residues when the consecutive U residues are present in the tracrRNA-crRNA complementarity region containing partial deletion.

As an example of tracrRNA to which modifications at the plurality of modification sites (MS) as described above have been applied, there is provided an engineered tracrRNA comprising the nucleotide sequence of any one of SEQ ID NOs: 87 to 132.

Specifically, the engineered tracrRNA of the present disclosure may comprise or consist of the nucleotide sequence of SEQ ID NO: 87 (MS1), SEQ ID NO: 88 (MS1/MS3-1), SEQ ID NO: 89 (MS1/MS3-2), SEQ ID NO: 90 (MS1/MS3-3), SEQ ID NO: 91 (MS1/MS4*-1), SEQ ID NO: 92 (MS1/MS4*-2), SEQ ID NO: 93 (MS1/MS4*-3), SEQ ID NO: 94 (MS1/MS5-1), SEQ ID NO: 95 (MS1/MS5-2), SEQ ID NO: 96 (MS1/MS5-3), SEQ ID NO: 97 (MS1/MS3-3/MS4*-1), SEQ ID NO: 98 (MS1/MS3-3/MS4*-2), SEQ ID NO: 99 (MS1/MS3-3/MS4*-3), SEQ ID NO: 100 (MS1/MS4*-2/MS5-1), SEQ ID NO: 101 (MS1/MS4*-2/MS5-2), SEQ ID NO: 102 (MS1/MS4*-2/MS5-3), SEQ ID NO: 103 (MS1/MS3-3/MS5-1), SEQ ID NO: 104 (MS1/MS3-3/MS5-2), SEQ ID NO: 105 (MS1/MS3-3/MS5-3), SEQ ID NO: 106 (MS1/MS3-3/MS4*-2/MS5-3), SEQ ID NO: 107 (mature form, MF), SEQ ID NO: 108 (MF/MS3-1), SEQ ID NO: 109 (MF/MS3-2), SEQ ID NO: 110 (MF/MS3-3), SEQ ID NO: 111 (MF/MS4-1), SEQ ID NO: 112 (MF/MS4-2), SEQ ID NO: 113 (MF/MS4-3), SEQ ID NO: 114 (MF/MS5-1), SEQ ID NO: 115 (MF/MS5-2), SEQ ID NO: 116 (MF/MS5-3), SEQ ID NO: 117 (MF/MS5), SEQ ID NO: 118 (MF/MS3-3/MS4-1), SEQ ID NO: 119 (MF/MS3-3/MS4-2), SEQ ID NO: 120 (MF/MS3-3/MS4-3), SEQ ID NO: 121 (MF/MS4-3/MS5-1), SEQ ID NO: 122 (MF/MS4-3/MS5-2), SEQ ID NO: 123 (MF/MS4-3/MS5-3), SEQ ID NO: 124 (MF/MS4-3/MS5-F), SEQ ID NO: 125 (MF/MS3-3/MS5-1), SEQ ID NO: 126 (MF/MS3-3/MS5-2), SEQ ID NO: 127 (MF/MS3-3/MS5-3), SEQ ID NO: 128 (MF/MS3-3/MS5), SEQ ID NO: 129 (MF/MS3-3/MS4-3/MS5-3), SEQ ID NO: 130 (MF/MS3-3/MS4-1/MS5), SEQ ID NO: 131 (MF/MS3-3/MS4-2/MS5), or SEQ ID NO: 132 (MF/MS3-3/MS4-3/MS5).

In some embodiments, exemplary sequences of the engineered tracrRNA, which has one or more modifications at any one or more of the modification sites selected from MS1, MS3, MS4, and MS5, are provided in Table 2.

TABLE 2

| | | SEQ ID |
|---|---|---|
| tracrRNA | Nucleotide sequence | NO |
| MS1 | CUUCACUGAUAAAGUGGAGAACCGCUUCACCAAAAGC | 87 |
| | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAAUUCAGUGCUCCUCU | |
| | CCAAUUCUGCACAA | |
| MS1/MS3-1 | GAUAAAGUGGAGAACCGCUUCACCAAAAGCUGUCCCU | 88 |
| | UAGGGGAUUAGAACUUGAGUGAAGGUGGGCUGCUUG | |
| | CAUCAGCCUAAUGUCGAGAAGUGCUUUCUUCGGAAAG | |
| | UAACCCUCGAAACAAAUUCAGUGCUCCUCUCCAAUUC | |
| | UGCACAA | |
| MS1/MS3-2 | UGGAGAACCGCUUCACCAAAAGCUGUCCCUUAGGGGA | 89 |
| | UUAGAACUUGAGUGAAGGUGGGCUGCUUGCAUCAGCC | |
| | UAAUGUCGAGAAGUGCUUUCUUCGGAAAGUAACCCUC | |
| | GAAACAAAUUCAGUGCUCCUCUCCAAUUCUGCACAA | |
| MS1/MS3-3 | ACCGCUUCACCAAAAGCUGUCCCUUAGGGGAUUAGAA | 90 |
| | CUUGAGUGAAGGUGGGCUGCUUGCAUCAGCCUAAUGU | |
| | CGAGAAGUGCUUUCUUCGGAAAGUAACCCUCGAAACA | |
| | AAUUCAGUGCUCCUCUCCAAUUCUGCACAA | |
| MS1/MS4*-1 | CUUCACUGAUAAAGUGGAGAACCGCUUCACCAAAAGC | 91 |
| | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAAUUCAGUGCUCCUCU | |
| | CCAAUUC | |
| MS1/MS4*-2 | CUUCACUGAUAAAGUGGAGAACCGCUUCACCAAAAGC | 92 |
| | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAAUUCAGUGCUCCUCU | |
| | C | |
| MS1/MS4*-3 | CUUCACUGAUAAAGUGGAGAACCGCUUCACCAAAAGC | 93 |
| | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAAUUCAGUGCU | |
| MS1/MS5-1 | CUUCACUGAUAAAGUGGAGAACCGCUUCACCAAAAGC | 94 |
| | UGUUUAGAUUAGAACUUGAGUGAAGGUGGGCUGCUU | |
| | GCAUCAGCCUAAUGUCGAGAAGUGCUUUCUUCGGAAA | |
| | GUAACCCUCGAAACAAAUUCAGUGCUCCUCUCCAAUU | |
| | CUGCACAA | |
| MS1/MS5-2 | CUUCACUGAUAAAGUGGAGAACCGCUUCACCAAAAGC | 95 |
| | UUAGGAACUUGAGUGAAGGUGGGCUGCUUGCAUCAG | |
| | CCUAAUGUCGAGAAGUGCUUUCUUCGGAAAGUAACCC | |
| | UCGAAACAAAUUCAGUGCUCCUCUCCAAUUCUGCACA | |
| | A | |
| MS1/MS5-3 | CUUCACUGAUAAAGUGGAGAACCGCUUCACCAAUUAG | 96 |
| | UUGAGUGAAGGUGGGCUGCUUGCAUCAGCCUAAUGUC | |
| | GAGAAGUGCUUUCUUCGGAAAGUAACCCUCGAAACAA | |
| | AUUCAGUGCUCCUCUCCAAUUCUGCACAA | |
| MS1/MS3- | ACCGCUUCACCAAAAGCUGUCCCUUAGGGGAUUAGAA | 97 |
| 3/MS4*-1 | CUUGAGUGAAGGUGGGCUGCUUGCAUCAGCCUAAUGU | |
| | CGAGAAGUGCUUUCUUCGGAAAGUAACCCUCGAAACA | |
| | AAUUCAGUGCUCCUCUCCAAUUC | |
| MS1/MS3- | ACCGCUUCACCAAAAGCUGUCCCUUAGGGGAUUAGAA | 98 |
| 3/MS4*-2 | CUUGAGUGAAGGUGGGCUGCUUGCAUCAGCCUAAUGU | |
| | CGAGAAGUGCUUUCUUCGGAAAGUAACCCUCGAAACA | |
| | AAUUCAGUGCUCCUCUC | |
| MS1/MS3- | ACCGCUUCACCAAAAGCUGUCCCUUAGGGGAUUAGAA | 99 |
| 3/MS4*-3 | CUUGAGUGAAGGUGGGCUGCUUGCAUCAGCCUAAUGU | |
| | CGAGAAGUGCUUUCUUCGGAAAGUAACCCUCGAAACA | |
| | AAUUCAGUGCU | |
| MS1/MS4*- | CUUCACUGAUAAAGUGGAGAACCGCUUCACCAAAAGC | 100 |
| 2/MS5-1 | UGUUUAGAUUAGAACUUGAGUGAAGGUGGGCUGCUU | |
| | GCAUCAGCCUAAUGUCGAGAAGUGCUUUCUUCGGAAA | |
| | GUAACCCUCGAAACAAAUUCAGUGCUCCUCUC | |
| MS1/MS4*- | CUUCACUGAUAAAGUGGAGAACCGCUUCACCAAAAGC | 101 |
| 2/MS5-2 | UUAGGAACUUGAGUGAAGGUGGGCUGCUUGCAUCAG | |
| | CCUAAUGUCGAGAAGUGCUUUCUUCGGAAAGUAACCC | |
| | UCGAAACAAAUUCAGUGCUCCUCUC | |
| MS1/MS4*- | CUUCACUGAUAAAGUGGAGAACCGCUUCACCAAUUAG | 102 |
| 2/MS5-3 | UUGAGUGAAGGUGGGCUGCUUGCAUCAGCCUAAUGUC | |
| | GAGAAGUGCUUUCUUCGGAAAGUAACCCUCGAAACAA | |
| | AUUCAGUGCUCCUCUC | |
| MS1/MS3- | ACCGCUUCACCAAAAGCUGUUUAGAUUAGAACUUGAG | 103 |
| 3/MS5-1 | UGAAGGUGGGCUGCUUGCAUCAGCCUAAUGUCGAGAA | |
| | GUGCUUUCUUCGGAAAGUAACCCUCGAAACAAAUUCA | |
| | GUGCUCCUCUCCAAUUCUGCACAA | |
| MS1/MS3- | ACCGCUUCACCAAAAGCUUAGGAACUUGAGUGAAGGU | 104 |
| 3/MS5-2 | GGGCUGCUUGCAUCAGCCUAAUGUCGAGAAGUGCUUU | |
| | CUUCGGAAAGUAACCCUCGAAACAAAUUCAGUGCUCC | |
| | UCUCCAAUUCUGCACAA | |
| MS1/MS3- | ACCGCUUCACCAAUUAGUUGAGUGAAGGUGGGCUGCU | 105 |
| 3/MS5-3 | UGCAUCAGCCUAAUGUCGAGAAGUGCUUUCUUCGGAA | |
| | AGUAACCCUCGAAACAAAUUCAGUGCUCCUCUCCAAU | |
| | UCUGCACAA | |
| MS1/MS3- | UGCAUCAGCCUAAUGUCGAGAAGUGCUUUCUUCGGAA | 106 |
| 3/MS4*-2/MS5-3 | ACCGCUUCACCAAUUAGUUGAGUGAAGGUGGGCUGCU | |
| | AGUAACCCUCGAAACAAAUUCAGUGCUCCUCUC | |
| Mature Form | CUUCACUGAUAAAGUGGAGAACCGCUUCACCAAAAGC | 107 |
| (MF) | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAAUUCAUUU | |
| MF/MS3-1 | GAUAAAGUGGAGAACCGCUUCACCAAAAGCUGUCCCU | 108 |
| | UAGGGGAUUAGAACUUGAGUGAAGGUGGGCUGCUUG | |
| | CAUCAGCCUAAUGUCGAGAAGUGCUUUCUUCGGAAAG | |
| | UAACCCUCGAAACAAAUUCAUUU | |
| MF/MS3-2 | UGGAGAACCGCUUCACCAAAAGCUGUCCCUUAGGGGA | 109 |
| | UUAGAACUUGAGUGAAGGUGGGCUGCUUGCAUCAGCC | |
| | UAAUGUCGAGAAGUGCUUUCUUCGGAAAGUAACCCUC | |
| | GAAACAAAUUCAUUU | |
| MF/MS3-3 | ACCGCUUCACCAAAAGCUGUCCCUUAGGGGAUUAGAA | 110 |
| | CUUGAGUGAAGGUGGGCUGCUUGCAUCAGCCUAAUGU | |
| | CGAGAAGUGCUUUCUUCGGAAAGUAACCCUCGAAACA | |
| | AAUUCAUUU | |
| MF/MS4-1 | CUUCACUGAUAAAGUGGAGAACCGCUUCACCAAAAGC | 111 |
| | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAAUUCAU | |
| MF/MS4-2 | CUUCACUGAUAAAGUGGAGAACCGCUUCACCAAAAGC | 112 |
| | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAAUUC | |
| MF/MS4-3 | CUUCACUGAUAAAGUGGAGAACCGCUUCACCAAAAGC | 113 |
| | UGUCCCUUAGGGGAUUAGAACUUGAGUGAAGGUGGG | |
| | CUGCUUGCAUCAGCCUAAUGUCGAGAAGUGCUUUCUU | |
| | CGGAAAGUAACCCUCGAAACAAA | |
| MF/MS5-1 | CUUCACUGAUAAAGUGGAGAACCGCUUCACCAAAAGC | 114 |
| | UGUUUAGAUUAGAACUUGAGUGAAGGUGGGCUGCUU | |
| | GCAUCAGCCUAAUGUCGAGAAGUGCUUUCUUCGGAAA | |
| | GUAACCCUCGAAACAAAUUCAUUU | |
| MF/MS5-2 | CUUCACUGAUAAAGUGGAGAACCGCUUCACCAAAAGC | 115 |
| | UUUAGAGAACUUGAGUGAAGGUGGGCUGCUUGCAUC | |
| | AGCCUAAUGUCGAGAAGUGCUUUCUUCGGAAAGUAAC | |
| | CCUCGAAACAAAUUCAUUU | |
| MF/MS5-3 | CUUCACUGAUAAAGUGGAGAACCGCUUCACCAAUUAG | 116 |
| | UUGAGUGAAGGUGGGCUGCUUGCAUCAGCCUAAUGUC | |
| | GAGAAGUGCUUUCUUCGGAAAGUAACCCUCGAAACAA | |
| | AUUCAUUU | |
| MF/MS5 | CUUCACUGAUAAAGUGGAGAACCGCUUCACUUAGAGU | 117 |
| | GAAGGUGGGCUGCUUGCAUCAGCCUAAUGUCGAGAAG | |
| | UGCUUUCUUCGGAAAGUAACCCUCGAAACAAAUUCAU | |
| | UU | |
| MF/MS3- | ACCGCUUCACCAAAAGCUGUCCCUUAGGGGAUUAGAA | 118 |
| 3/MS4-1 | CUUGAGUGAAGGUGGGCUGCUUGCAUCAGCCUAAUGU | |
| | CGAGAAGUGCUUUCUUCGGAAAGUAACCCUCGAAACA | |
| | AAUUCAU | |
| MF/MS3- | ACCGCUUCACCAAAAGCUGUCCCUUAGGGGAUUAGAA | 119 |
| 3/MS4-2 | CUUGAGUGAAGGUGGGCUGCUUGCAUCAGCCUAAUGU | |
| | CGAGAAGUGCUUUCUUCGGAAAGUAACCCUCGAAACA | |
| | AAUUC | |
| MF/MS3- | ACCGCUUCACCAAAAGCUGUCCCUUAGGGGAUUAGAA | 120 |
| 3/MS4-3 | CUUGAGUGAAGGUGGGCUGCUUGCAUCAGCCUAAUGU | |
| | CGAGAAGUGCUUUCUUCGGAAAGUAACCCUCGAAACA | |
| | AA | |
| MF/MS4- | CUUCACUGAUAAAGUGGAGAACCGCUUCACCAAAAGC | 121 |
| 3/MS5-1 | UGUUUAGAUUAGAACUUGAGUGAAGGUGGGCUGCUU | |
| | GCAUCAGCCUAAUGUCGAGAAGUGCUUUCUUCGGAAA | |
| | GUAACCCUCGAAACAAA | |
| MF/MS4- | CUUCACUGAUAAAGUGGAGAACCGCUUCACCAAAAGC | 122 |
| 3/MS5-2 | UUUAGAGAACUUGAGUGAAGGUGGGCUGCUUGCAUC | |
| | AGCCUAAUGUCGAGAAGUGCUUUCUUCGGAAAGUAAC | |
| | CCUCGAAACAAA | |
| MF/MS4- | CUUCACUGAUAAAGUGGAGAACCGCUUCACCAAUUAG | 123 |
| 3/MS5-3 | UUGAGUGAAGGUGGGCUGCUUGCAUCAGCCUAAUGUC | |
| | GAGAAGUGCUUUCUUCGGAAAGUAACCCUCGAAACAA | |
| | A | |
| MF/MS4-3/MS5 | CUUCACUGAUAAAGUGGAGAACCGCUUCACUUAGAGU | 124 |
| | GAAGGUGGGCUGCUUGCAUCAGCCUAAUGUCGAGAAG | |
| | UGCUUUCUUCGGAAAGUAACCCUCGAAACAAA | |
| MF/MS3- | ACCGCUUCACCAAAAGCUGUUUAGAUUAGAACUUGAG | 125 |
| 3/MS5-1 | UGAAGGUGGGCUGCUUGCAUCAGCCUAAUGUCGAGAA | |
| | GUGCUUUCUUCGGAAAGUAACCCUCGAAACAAAUUCA | |
| | UUU | |
| MF/MS3- | ACCGCUUCACCAAAAGCUUUAGAGAACUUGAGUGAAG | 126 |
| 3/MS5-2 | GUGGGCUGCUUGCAUCAGCCUAAUGUCGAGAAGUGCU | |
| | UUCUUCGGAAAGUAACCCUCGAAACAAAUUCAUUU | |
| MF/MS3- | ACCGCUUCACCAAUUAGUUGAGUGAAGGUGGGCUGCU | 127 |
| 3/MS5-3 | UGCAUCAGCCUAAUGUCGAGAAGUGCUUUCUUCGGAA | |
| | AGUAACCCUCGAAACAAAUUCAUUU | |
| MF/MS3-3/MS5 | ACCGCUUCACUUAGAGUGAAGGUGGGCUGCUUGCAUC | 128 |
| | AGCCUAAUGUCGAGAAGUGCUUUCUUCGGAAAGUAAC | |
| | CCUCGAAACAAAUUCAUUU | |
| MF/MS3- | UGCAUCAGCCUAAUGUCGAGAAGUGCUUUCUUCGGAA | 129 |
| 3/MS4-3/MS5-3 | ACCGCUUCACCAAUUAGUUGAGUGAAGGUGGGCUGCU | |
| | AGUAACCCUCGAAACAAA | |
| MF/MS3- | ACCGCUUCACUUAGAGUGAAGGUGGGCUGCUUGCAUC | 130 |
| 3/MS4-1/MS5 | AGCCUAAUGUCGAGAAGUGCUUUCUUCGGAAAGUAAC | |
| | CCUCGAAACAAAUUCAU | |
| MF/MS3- | ACCGCUUCACUUAGAGUGAAGGUGGGCUGCUUGCAUC | 131 |
| 3/MS4-2/MS5 | AGCCUAAUGUCGAGAAGUGCUUUCUUCGGAAAGUAAC | |
| | CCUCGAAACAAAUUC | |
| MF/MS3- | ACCGCUUCACUUAGAGUGAAGGUGGGCUGCUUGCAUC | 132 |
| 3/MS4-3/MS5 | AGCCUAAUGUCGAGAAGUGCUUUCUUCGGAAAGUAAC | |
| | CCUCGAAACAAA | |

In addition, as an example of crRNA to which modifications at the plurality of modification sites (MS) as described above have been applied, there is provided an engineered crRNA comprising the nucleotide sequence of any one of SEQ ID NOs: 133 to 148.

Specifically, the engineered crRNA of the present disclosure may comprise or consist of the nucleotide sequence of SEQ ID NO: 133 (MS1), SEQ ID NO: 134 (MS1/MS4*-1), SEQ ID NO: 135 (MS1/MS4*-2), SEQ ID NO: 136 (MS1/MS4*-3), SEQ ID NO: 137 (mature form; MF), SEQ ID NO: 138 (MF/MS4-1), SEQ ID NO: 139 (MF/1M54-2), SEQ ID NO: 140 (MF/MS4-3), SEQ ID NO: 141 (MS1/MS2), SEQ ID NO: 142 (MS1/MS2/MS4*-1), SEQ ID NO: 143 (MS1/MS2/MS4*-2), SEQ ID NO: 144 (MS1/MS2/MS4*-3), SEQ ID NO: 145 (MF/MS2), SEQ ID NO: 146 (MF/MS2/1M54-1), SEQ ID NO: 147 (MF/MS2/MS4-2), or SEQ ID NO: 148 (MF/MS2/MS4-3).

In some embodiments, exemplary sequences of the engineered crRNA, which has one or more modifications at any one or more modification sites selected from MS1, MS2, and MS4, are provided in Table 3.

TABLE 3

| | | SEQ ID |
|---|---|---|
| crRNA | Nucleotide sequence | NO |
| MS1 | GUUGCAGAACCCGAAUAGAGCAAUGAAGGAAUGCAAC | 133 |
| MS1/MS4*-1 | GAACCCGAAUAGAGCAAUGAAGGAAUGCAAC | 134 |
| MS1/MS4*-2 | GAAUAGAGCAAUGAAGGAAUGCAAC | 135 |
| MS1/MS4*-3 | AGCAAUGAAGGAAUGCAAC | 136 |
| MF | GAAUGAAGGAAUGCAAC | 137 |
| MF/MS4-1 | AUGAAGGAAUGCAAC | 138 |
| MF/MS4-2 | GAAGGAAUGCAAC | 139 |
| MF/MS4-3 | GGAAUGCAAC | 140 |
| MS1/MS2 | GUUGCAGAACCCGAAUAGAGCAAUGAAGGAAUGCAAC | 141 |
| | NNNNNNNNNNNNNNNNNNNNUUUUAUUUUUU | |
| MS1/MS2/MS4*- | GAACCCGAAUAGAGCAAUGAAGGAAUGCAACNNNNN | 142 |
| 1 | NNNNNNNNNNNNNNNUUUUAUUUUUU | |
| MS1/MS2/MS4*- | GAAUAGAGCAAUGAAGGAAUGCAACNNNNNNNNNNN | 143 |
| 2 | NNNNNNNNNUUUUAUUUUUU | |
| MS1/MS2/MS4*- | AGCAAUGAAGGAAUGCAACNNNNNNNNNNNNNNNNN | 144 |
| 3 | NNNUUUUAUUUUUU | |
| MF/MS2 | GAAUGAAGGAAUGCAACNNNNNNNNNNNNNNNNNNN | 145 |
| | NUUUUAUUUUUU | |
| MF/MS2/MS4-1 | AUGAAGGAAUGCAACNNNNNNNNNNNNNNNNNNNNU | 146 |
| | UUUAUUUUUU | |
| MF/MS2/MS4-2 | GAAGGAAUGCAACNNNNNNNNNNNNNNNNNNNNUUU | 147 |
| | UAUUUUUU | |
| MF/MS2/MS4-3 | GGAAUGCAACNNNNNNNNNNNNNNNNNNNNUUUUAU | 148 |
| | UUUUU | |

In Table 3, indication of a guide sequence (spacer) is omitted from all crRNA sequences unless necessary, and the sequence indicated by ‘NNNNNNNNNNNNNNNNNNNN’ indicates any guide sequence (spacer) that can hybridize to a target sequence in a target gene. The guide sequence may be appropriately designed by those skilled in the art depending on a desired target gene and/or a target sequence in the target gene as described above, and therefore is not limited to a specific sequence of a particular length. In another embodiment, the engineered gRNA may comprise tracrRNA comprising or consisting of any one nucleotide sequence selected from the group consisting of SEQ ID NOs: 87 to 132; and crRNA comprising or consisting of any one nucleotide sequence selected from the group consisting of SEQ ID NOs: 133 to 148.

In another embodiment, when the engineered gRNA of the present disclosure is in the form of a single guide RNA (sgRNA), the engineered sgRNA may be sgRNA comprising or consisting of any one nucleotide sequence selected from the group consisting of SEQ ID NOs: 149 to 186.

Specifically, the engineered sgRNA may be sgRNA of SEQ ID NO: 149 comprising a modification at MS1, sgRNA of SEQ ID NO: 150 comprising modifications at MS1/MS2, sgRNA of SEQ ID NO: 151 comprising modifications at MS1/MS2/MS3, sgRNA of SEQ ID NO: 152 comprising modifications at MS2/MS3/MS4, or sgRNA of SEQ ID NO: 153 comprising modifications at MS2/MS3/MS4/MS5.

In another specific example, the engineered sgRNA may be sgRNA comprising or consisting of the nucleotide sequence of SEQ ID NO: 154 (MS1/MS3-1), SEQ ID NO: 155 (MS1/MS3-2), SEQ ID NO: 156 (MS1/MS3-3), SEQ ID NO: 157 (MS1/MS4*-1), SEQ ID NO: 158 (MS1/MS4*-2), SEQ ID NO: 159 (MS1/MS4*-3), SEQ ID NO: 160 (MS1/MS5-1), SEQ ID NO: 161 (MS1/MS5-2), SEQ ID NO: 162 (MS1/MS5-3), SEQ ID NO: 163 (MS1/MS2/MS4*-2), SEQ ID NO: 164 (MS1/MS3-3/MS4*-2), SEQ ID NO: 165 (MS1/MS2/MS5-3), SEQ ID NO: 166 (MS1/MS3-3/MS5-3), SEQ ID NO: 167 (MS1/MS4*-2/MS5-3), SEQ ID NO: 168 (MS1/MS2/MS3-3/MS4*-2), SEQ ID NO: 169 (MS1/MS2/MS3-3/MS5-3), SEQ ID NO: 170 (MS1/MS2/MS4*-2/MS5-3), SEQ ID NO: 171 (MS1/MS3-3/MS4*-2/MS5-3), or SEQ ID NO: 172 (MS1/MS2/MS3-3/MS4*-2/MS5-3).

In addition, the sgRNA may be sgRNA comprising or consisting of the nucleotide sequence of SEQ ID NO: 173, which is a mature form (abbreviated as MF) of sgRNA.

In another embodiment, there is provided an exemplary sgRNA which comprises partial modification of the nucleotide sequence of the MF sgRNA. Specifically, the MF sgRNA may be an sgRNA comprising or consisting of the nucleotide sequence of SEQ ID NO: 174 (MS3-1), SEQ ID NO: 175 (MS3-2), SEQ ID NO: 176 (MS3-3), SEQ ID NO: 177 (MS4-1), SEQ ID NO: 178 (MS4-2), SEQ ID NO: 179 (MS4-3), SEQ ID NO: 180 (MS5-1), SEQ ID NO: 181 (MS5-2), SEQ ID NO: 182 (MS5-3), SEQ ID NO: 183 (MS3-3/MS4-3), SEQ ID NO: 184 (MS3-3/MS5-3), SEQ ID NO: 185 (MS4-3/MS5-3), or SEQ ID NO: 186 (MS3-3/MS4-3/MS5-3).

In a preferred embodiment, the engineered sgRNA may consist of the nucleotide sequence of SEQ ID NO: 151 (Cas12f_ge3.0), SEQ ID NO: 152 (Cas12f_ge4.0), or SEQ ID NO: 153 (Cas12f_ge4.1).

### (7) Additional Sequence

The engineered tracrRNA of the present disclosure may optionally further comprise an additional sequence. The additional sequence may be located at the 3′-end of the engineered tracrRNA. In addition, the additional sequence may be located at the 5′-end of the engineered tracrRNA. For example, the additional sequence may be located at the 5′-end of the first stem region.

The additional sequence may consist of 1 to 40 nucleotides. In an embodiment, the additional sequence may be any nucleotide sequence or a randomly arranged nucleotide sequence. For example, the additional sequence may be 5′-AUAAAGGUGA-3′ (SEQ ID NO: 187).

In addition, the additional sequence may be a known nucleotide sequence. For example, the additional sequence may be a hammerhead ribozyme nucleotide sequence. Here, the hammerhead ribozyme nucleotide sequence may be 5′-CUGAUGAGUCCGUGAGGACGAAACGAGUAAGCUCGUC-3′ (SEQ ID NO: 188) or 5′-CUGCUCGAAUGAGCAAAGCAGGAGUGCCUGAGUAGUC-3′ (SEQ ID NO: 189). The sequences listed above are merely examples, and the additional sequence is not limited thereto.

### (8) Chemical Modification

In some embodiments, the engineered tracrRNA or engineered crRNA included in the engineered gRNA may have chemical modification in at least one nucleotide, if necessary. Here, the chemical modification may be a modification in various covalent bonds that may occur in a nucleotide base and/or sugar portion.

For example, the chemical modification may be methylation, halogenation, acetylation, phosphorylation, phosphorothioate (PS) linkage, locked nucleic acid (LNA), 2′-O-methyl 3′phosphorothioate (MS), or 2′-O-methyl 3′thioPACE (MSP). The above example is a simple example and the modification is not limited thereto.

In a case of using the hypercompact gene editing system comprising a complex of the engineered gRNA with engineered Cas12f1 (CWCas12f1 or UnCas12f1) of the present disclosure, indel efficiency for a target gene or target nucleic acid in a cell is significantly improved compared to a case of using the guide RNA or Cas12f1 found in nature.

Above all, the engineered gRNA may involve optimized length for high efficiency and resulting cost reduction in gRNA synthesis, creation of additional space or capacity in a case of being inserted into a viral vector, normal expression of tracrRNA, increased expression of operable gRNA, increased gRNA stability, increased stability of complex of gRNA with nucleic acid editing protein, induction of formation of complex of gRNA with nucleic acid editing protein at high efficiency, increased cleavage efficiency of target nucleic acid by hypercompact nucleic acid editing system comprising complex of gRNA with nucleic acid editing protein, and increased homology-directed repair efficiency for target nucleic acid caused by such a system. Accordingly, in a case of using the above-described engineered gRNA for Cas12f1 or an engineered Cas12f1 protein, it is possible to overcome the limitations of the above-mentioned prior art, thereby cleaving or editing a gene with high efficiency in a cell.

In addition, the engineered gRNA has a short length compared to gRNA found in nature, and thus has high applicability in the field of gene editing technology. Using the engineered gRNA, the hypercompact gene editing system comprising a complex of the gRNA with nucleic acid editing protein has advantages of being very small in size and having excellent editing efficiency, which allows the system to be utilized in various gene editing technologies.

### 3. Factors Capable of Reducing Non-Homologous End Joining Activity

As disclosed herein, the Cas12f1 system may further comprise a factor capable of reducing non-homologous end joining activity, for example, a molecule that inhibits expression of a gene involved in non-homologous end joining, or a nucleic acid encoding the molecule. Without being bound by any particular theory, for example, reduction of NHEJ activity may result in promotion of a homology-directed repair (HDR) mediated pathway. The inhibitory molecule may be used to decrease NHEJ activity or increase or decrease HDR activity. In an embodiment, the present inventors have confirmed that addition of a molecule, which inhibits expression of a gene involved in non-homologous end joining to the Cas12f1 system of the present disclosure for deleting a nucleic acid segment comprising exon 51 in a dystrophin gene, significantly increases deletion efficiency of the segment comprising exon 51.

In an embodiment, the inhibitory molecule may be a small molecule or an inhibitory nucleic acid. The inhibitory molecule may be, for example, but is not limited to, an interfering nucleic acid (for example, short interfering RNA (siRNA), double-stranded RNA (dsRNA), micro-RNA (miRNA), short hairpin RNA (shRNA) specific for a gene transcript) or an antisense oligonucleotide.

In another embodiment, the inhibitory molecule may be targeted to enzymes involved in NHEJ, HDR, or upstream regulation thereof by post translational modification, for example, through phosphorylation, ubiquitination, and/or sumoylation.

In mammalian cells, the “canonical” or “classical” NHEJ pathway (C-NHEJ) requires several factors, including DNA-PK, Ku70-80, Artemis, ligase IV (Lig4), XRCC4, CLF, and Pol p, to repair double-strand breaks (see Kasparek & Humphrey Seminars in Cell & Dev. Biol. 22:886-897, 2011).

In an embodiment, to inhibit the C-NHEJ pathway in a cell, the Cas12f1 system of the present disclosure may be modified to reduce or eliminate expression or activity of a factor involved in the NHEJ pathway. For example, the Cas12f1 system may further comprise a factor capable of reducing or eliminating expression or activity of one or more selected from the group consisting of MRE11, RAD50, NBS1, DNA-PK, CtIP, Ku70, Ku80, Artemis (DCLRE1C), Ligase IV (Lig4), PNKP, XRCC4, XLF (XRCC4-like factor), ATM (ATM Serine/Threonine Kinase), CHK1/CHK2, CURLY LEAF (CLF), and Pol Mu (POLM).

In mammals, in addition to C-NHEJ, an alternative NHEJ (A-NHEJ) pathway exists, which is known to require different factors.

In another embodiment, to inhibit the A-NHEJ pathway in a cell, the Cas12f1 system of the present disclosure may be modified to reduce or eliminate expression or activity of a factor involved in the NHEJ pathway. For example, the Cas12f1 system may further comprise a factor capable of reducing or eliminating expression or activity of one or more selected from the group consisting of XRCC1, PARP (for example, PARP1), Lig1, and Lig3.

In an embodiment, the gene involved in non-homologous end joining may be one or more selected from the group consisting of ATM1, XRCC4, XLF, XRCC6, LIG4, and DCLRE1C.

In another embodiment, the gene involved in non-homologous end joining may be one or more selected from the group consisting of XRCC6 and DCLRE1C.

In an embodiment, the inhibitory molecule may be shRNA, siRNA, miRNA, or an antisense oligonucleotide.

In another embodiment, the inhibitory molecule may be shRNA.

In yet another embodiment, the shRNA molecule may be a molecule that inhibits expression of one or more genes selected from the group consisting of XRCC6 and DCLRE1C. Specifically, the shRNA molecule may be one or more selected from the group consisting of shXRCC6 and shDCLRE1C.

In still yet another embodiment, the shRNA molecule may be one or more selected from the group consisting of SEQ ID NOs: 360 to 389 and SEQ ID NO: 403.

In still yet another embodiment, the shRNA molecule may be one or more selected from the group consisting of SEQ ID NOs: 375 to 379 and SEQ ID NOs: 385 to 389.

In an embodiment, the system or composition disclosed herein may comprise two or more molecules that inhibit expression of genes involved in the non-homologous end joining pathway, or nucleic acids (nucleic acid constructs) encoding the molecules.

In an embodiment, the two or more inhibitory molecules may each inhibit expression of identical or different genes.

### 4. Nucleic Acid or Polynucleotide Encoding Each Component of Cas12f1 System

Since each component of the gene editing system provided in the present disclosure is intended to be expressed within a cell, according to another aspect, there is provided a nucleic acid or polynucleotide encoding each component of the gene editing system. The nucleic acid or polynucleotide may be a synthetic nucleotide sequence.

Specifically, the nucleic acid or polynucleotide is provided as a nucleotide sequence encoding a nucleic acid editing protein (engineered endonuclease), a guide RNA, and/or a molecule that inhibits expression of a gene involved in non-homologous end joining, included in a gene editing system to be expressed. In an embodiment, the nucleotide sequence may be DNA or RNA (for example, mRNA). The nucleic acid or polynucleotide encoding each component of the gene editing system is disclosed herein as a representative example, or the nucleotide sequence thereof may be readily determined by those skilled in the art by referring to the specific sequence of each component.

In an embodiment, the nucleic acid or polynucleotide may comprise a human codon-optimized nucleotide sequence encoding a Cas12f1 protein. The term “codon optimization” refers to a process of modifying a native nucleic acid sequence for enhanced expression in a cell of interest by replacing at least one codon in the native sequence with a codon that is used more frequently or most frequently in a gene of the target cell, while maintaining its native amino acid sequence. Different species have specific biases for specific codons for specific amino acids, and codon bias (differences in codon usage between organisms) is often correlated with translation efficiency of an mRNA, which is considered to be dependent on the nature of codons being translated and availability of specific tRNA molecules. Predominance of tRNA selected in a cell generally reflects the most frequently used codon in peptide synthesis. Thus, genes may be tailored for optimal gene expression in a given organism based on codon optimization.

For example, the nucleic acid encoding the human codon optimized CWCas12f1 protein or a variant thereof may comprise or consist of a sequence selected from SEQ ID NOs: 6 to 9. In addition, the nucleic acid encoding the human codon optimized UnCas12f1 protein may comprise or consist of the sequence of SEQ ID NO: 10.

In another embodiment, the nucleic acid or polynucleotide may be DNA or RNA that exists in nature, or may be a modified nucleic acid in which a chemical modification has occurred in at least a part of the nucleic acid or polynucleotide. For example, the nucleic acid or polynucleotide may be one in which one or more nucleotides have been chemically modified. Here, the chemical modification may include any modification of nucleic acids known to those skilled in the art.

### IV. Vector System for Editing Dystrophin Gene

As disclosed herein, there is provided a vector system for editing or modifying a dystrophin gene (for example, a human dystrophin gene). Since the disclosed vector system allows each component of the above-described Cas12f1 system to be expressed in a cell, the nucleic acid construct (for example, nucleotide sequence) included in the vector system comprises at least one nucleotide sequence encoding each component of the Cas12f1 system. In addition, since the disclosed vector system allows each component of the Cas12f1 system to be expressed in a cell, all effects and advantages that are achieved by the Cas12f1 system are applied as is.

In the disclosed vector system, each nucleic acid construct is capable of expressing each component of the Cas12f1 system in a cell. The vector system enables editing of the dystrophin gene (for example, deletion of a segment comprising exon 51) in a cell.

In the vector system disclosed herein, for the nucleotide sequence of each nucleic acid construct and the components expressed thereby, see the section “III. CRISPR/Cas system for editing dystrophin gene.”

In order to use the Cas12f1 system disclosed herein for editing the dystrophin gene (for example, deletion of a segment comprising exon 51), a method may be used in which one or more vectors comprising nucleotide sequences encoding respective components of the above-described Cas21f1 system may be introduced directly or through an appropriate delivery means, such as a virus, into a target cell, and the respective components of the gene editing system are allowed to be expressed in the target cell. Preferably, for editing the dystrophin gene (for example, deletion of a segment comprising exon 51), nucleotide sequences encoding respective components of the above-described Cas21f1 system may be operably linked and contained in a single vector.

In an embodiment, the nucleotide sequences encoding one or more components of the above-described Cas21f1 system may be present in two or more vectors.

In another embodiment, the nucleotide sequences encoding one or more components of the above-described Cas21f1 system may be present in a single vector.

In addition, the vector system of the present disclosure may comprise, in addition to the above-described components of the Cas21f1 system, a nucleotide sequence encoding an additional expression element that is desired to be expressed as needed by those skilled in the art. For example, the additional expression element may be a tag. Specifically, the additional expression element may be a herbicide resistance gene such as glyphosate, glufosinate ammonium, or phosphinothricin, or an antibiotic resistance gene such as ampicillin, kanamycin, G418, bleomycin, hygromycin, or chloramphenicol.

In another embodiment, the vector system needs to comprise one or more regulatory and/or control components so that it is directly expressed in a cell. Specifically, the regulatory and/or control components may include, but are not limited to, a promoter, an enhancer, an intron, a polyadenylation signal, a Kozak consensus sequence, an internal ribosome entry site (IRES), a splice acceptor, a 2A sequence, and/or a replication origin. The replication origin may be, but is not limited to, an f1 origin of replication, an SV40 origin of replication, a pMB1 origin of replication, an adeno origin of replication, an AAV origin of replication, and/or a BBV origin of replication.

In another embodiment, in order to express, in a cell, the nucleic acid sequence encoding the nucleic acid editing system of the present disclosure included in the vector system, a promoter sequence may need to be operably linked to the sequence encoding each component so that an RNA transcription factor can be activated in the cell. The promoter sequence may be designed differently depending on the corresponding RNA transcription factor or expression environment, and is not limited as long as it can properly express the components of the nucleic acid editing system (TaRGET system) of the present disclosure in a cell.

For example, the promoter sequence may be a promoter that promotes transcription of RNA polymerase RNA Pol I, Pol II, or Pol III. Specifically, the promoter may be one of U6 promoter, EFS promoter, EF1-α promoter, H1 promoter, 7SK promoter, CMV promoter, LTR promoter, Ad MLP promoter, HSV promoter, SV40 promoter, CBA promoter, or RSV promoter.

In another embodiment, when a sequence of the vector comprises the promoter sequence, transcription of a sequence operably linked to the promoter is induced by an RNA transcription factor, and the vector may comprise a termination signal that induces termination of transcription of the RNA transcription factor. The termination signal may vary depending on the type of the promoter sequence. Specifically, when the promoter is a U6 or H1 promoter, the promoter recognizes a TTTTT (T5) or TTTTTT (T6) sequence, which is a thymidine (T) repeat sequence, as a termination signal.

The sequence of the engineered guide RNA provided herein may comprise a U-rich tail sequence at its 3′-end. Accordingly, the sequence encoding the engineered guide RNA comprises a T-rich sequence corresponding to the U-rich tail sequence at its 3′-end. As described above, some promoter sequences recognize a thymidine (T) repeat sequence, for example, a sequence consisting of five or more consecutive thymidine (T) residues, as a termination signal, and therefore, in some cases, the T-rich sequence may be recognized as a termination signal. In other words, when the vector sequence provided herein comprises a sequence encoding the engineered guide RNA, a sequence encoding the U-rich tail sequence included in the engineered gRNA sequence may be used as a termination signal.

In an embodiment, when the vector sequence comprises a U6 or H1 promoter sequence and a sequence encoding the engineered guide RNA operably linked thereto, a sequence portion that encodes the U-rich tail sequence included in the guide RNA sequence may be recognized as a termination signal. Specifically, the U-rich tail sequence may comprise a sequence consisting of five or more consecutive uridine (U) residues.

In an embodiment, the vector may be a viral vector. Specifically, the viral vector may be at least one selected from the group consisting of a retrovirus vector, a lentivirus vector, an adenovirus vector, an adeno-associated virus vector, a vaccinia virus vector, a poxvirus vector, a herpes simplex virus vector, and a phagemid vector. Preferably, the viral vector may be an adeno-associated virus vector (AAV). In addition, the viral vector includes, but is not limited to, a SIN lentivirus vector, a retrovirus vector, a foamy virus vector, an adenovirus vector, an adeno-associated virus (AAV) vector, a hybrid vector and/or a plasmid transposon (for example, the Sleeping Beauty transposon system), or an integrase-based vector system.

In another embodiment, the vector may be a non-viral vector. Specifically, the non-viral vector may be at least one selected from the group consisting of, but not limited to, plasmid, naked DNA, DNA complex, mRNA (transcript), and amplicon. For example, the plasmid may be selected from the group consisting of pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, and pUC19.

The term “naked DNA” refers to DNA (for example, histone-free DNA) that encodes a protein, such as Cas12f1 or a variant thereof of the present disclosure, cloned into a suitable expression vector (for example, plasmid) in an appropriate orientation for expression.

The term “amplicon,” when used with respect to a nucleic acid, means a product obtained by copying the nucleic acid, wherein the product has a nucleotide sequence that is identical with or complementary to at least a portion of the nucleotide sequence of the nucleic acid. For example, an amplicon may be produced by any of a variety of amplification methods, which use a nucleic acid or an amplicon thereof as a template, including polymerase extension, polymerase chain reaction (PCR), rolling circle amplification (RCA), multi-displacement amplification (MDA), ligation extension, or ligation chain reaction. The amplicon may be a nucleic acid molecule having a single copy of a particular nucleotide sequence (for example, a PCR product) or multiple copies of the nucleotide sequence (for example, a concatemeric product of RCA).

The vector disclosed herein may be designed in the form of a linear or circular vector. In a case where the vector is a linear vector, RNA transcription is terminated at the 3′-end even if a sequence of the linear vector does not separately comprise a termination signal. However, in a case where the vector is a circular vector, RNA transcription is not terminated unless a sequence of the circular vector separately comprises a termination signal. Therefore, when using a circular vector, a termination signal corresponding to a transcription factor related to each promoter sequence has to be included in order for the vector to express an intended target.

In an embodiment, the viral vector or non-viral vector may be delivered by a delivery system such as liposomes, polymeric nanoparticles (for example, lipid nanoparticles), oil-in-water nanoemulsions, or combinations thereof, or in the form of a virus.

### V. Virus Produced by Vector System of Present Disclosure

There is provided a virus particle produced by the vector system disclosed herein.

In an embodiment, the viral vector may be, for example, at least one selected from the group consisting of a retrovirus vector, a lentivirus vector, an adenovirus vector, an adeno-associated virus vector, a vaccinia virus vector, a poxvirus vector, a herpes simplex virus vector, and a phagemid vector. Preferably, the viral vector may be an adeno-associated virus vector.

In another embodiment, the virus may be selected from the group consisting of a retrovirus, a lentivirus, an adenovirus, an adeno-associated virus, a vaccinia virus, a poxvirus, a herpes simplex virus, and a phage.

In yet another embodiment, the phage may be selected from the group consisting of λgt4λB, λ-charon, λΔz1, and M13.

In order to efficiently deliver the nucleic acid editing system of the present disclosure into a target cell or target site via a virus, in particular, an adeno-associated virus (AAV), it is important to design a size of the nucleotide sequence encoding all components of the editing system to be within 4.7 kb that is a packaging limit of AAV. This has an advantage in that in a case where the Cas12f1 system of the present disclosure is used, a very small size of the hypercompact nucleic acid editing protein and two engineered gRNAs included in the system allows sufficient packaging by AAV even if an additional regulatory molecule is further included.

### VI. Composition for Editing Dystrophin Gene

As disclosed herein, there is provided a composition comprising each component of the above-described system, one or more vectors of the above-described vector system, or the above-described virus. The disclosed composition may be a pharmaceutical composition.

In an embodiment, the pharmaceutical composition may be for editing a dystrophin gene (for example, deleting a segment comprising exon 51 in a dystrophin gene). In addition, the pharmaceutical composition may be for treating or delaying onset or progression of Duchenne muscular dystrophy.

In an embodiment, the pharmaceutical composition may be formulated according to the mode of administration to be used. For example, in a case where the pharmaceutical composition is an injectable pharmaceutical composition, it may be desirable to use an isotonic agent. An additive for isotonicity may generally include sodium chloride, dextrose, mannitol, sorbitol, and lactose. In an embodiment, isotonic solutions such as phosphate buffered saline are preferred. A stabilizer may include gelatin and albumin. In an embodiment, a vasoconstrictor is added to the formulation.

In another embodiment, the composition may further comprise a pharmaceutically acceptable excipient. The pharmaceutically acceptable excipient may be a functional molecule that acts as a vehicle, an adjuvant, a carrier, or a diluent. The pharmaceutically acceptable excipient may be a gene transfer enhancer (which may include a surfactant) such as an immune stimulating complex (ISCOMS), Freund's incomplete adjuvant, a LPS analogue (including monophosphoryl lipid A), a muramyl peptide, a quinone analogue, a vesicle, squalene, hyaluronic acid, a lipid, a liposome, a calcium ion, a viral protein, a polyanion, a polycation, or a nanoparticle, or another known gene transfer facilitating agent.

In another embodiment, the composition may comprise a gene transfer enhancer. The gene transfer enhancer may be a polyanion, a polycation (including poly-L-glutamic acid (LGS)), or a lipid. The gene transfer enhancer is poly-L-glutamic acid, and more preferably, the poly-L-glutamic acid may be present in the composition for genome editing of skeletal muscle or cardiac muscle at a concentration of less than 6 mg/ml. The gene transfer enhancer may also include a surfactant, such as an immune stimulating complex (ISCOMS), Freund's incomplete adjuvant, a LPS analogue (including monophosphoryl lipid A), a muramyl peptide, a quinone analogue, and a vesicle, such as squalene, and hyaluronic acid may also be used.

In an embodiment, the composition comprising one or more vectors included in the above-described vector system may comprise a gene transfer enhancer, such as a lipid, a liposome (including lecithin liposomes, or other liposomes known in the art), a DNA-liposome mixture, a calcium ion, a viral protein, a polyanion, a polycation, or a nanoparticle, or another known gene transfer enhancer. Preferably, the gene transfer enhancer is a polyanion, a polycation (for example, poly-L-glutamic acid (LGS)), or a lipid.

An actual dosage of the (pharmaceutical) composition may vary greatly depending on various factors, such as the choice of vector, the target cell, organism, or tissue, the condition of the subject to be treated, the degree of transformation/modification sought, the route of administration, the method of administration, the form of transformation/modification sought, and the like. The administration may be performed by a route of administration selected from subretinal administration, subcutaneous administration, intradermal administration, intraocular administration, intravitreal administration, intratumoral administration, intranodal administration, intramedullary administration, intramuscular administration, intravenous administration, intralymphatic administration, and intraperitoneal administration. The pharmaceutical composition may further comprise a carrier (for example, water, saline, ethanol, glycerol, lactose, sucrose, calcium phosphate, gelatin, dextran, agar, pectin, peanut oil, sesame oil, and the like), a diluent, a pharmaceutically acceptable carrier (for example, phosphate buffered saline), a pharmaceutically acceptable excipient, and/or other compounds known in the art.

For example, delivery for treatment of a disease may be via AAV. A therapeutically effective dosage for in vivo delivery of AAV to a human may be a saline solution in a range of about 20 ml to about 50 ml containing about 1×10¹⁰ to about 1×10¹⁰⁰ AAV per ml of solution. The dosage may be adjusted to balance the therapeutic benefit against any adverse effects.

### VII. Method for Editing Dystrophin Gene in Cell

As disclosed herein, there is provided a method for editing a dystrophin gene using the Cas12f1 system, vector system, composition, or virus of the present disclosure. Specifically, editing a dystrophin gene may involve generating deletion of a segment comprising exon 51 in the dystrophin gene.

In an embodiment, a length of the segment comprising exon 51 may be from 230 bp to 9 kbp, for example, from 230 bp to 8 kbp, from 230 bp to 7 kbp, from 230 bp to 6 kbp, from 230 bp to 5 kbp, from 230 bp to 4 kbp, from 230 bp to 3 kbp, from 230 bp to 2 kbp, from 230 bp to 1 kbp; from 1 kbp to 9 kbp, from 2 kbp to 8 kbp, from 3 kbp to 7 kbp, from 4 kbp to 6 kbp; from 230 bp to 1000 bp, from 300 bp to 1000 bp, from 400 bp to 900 bp, from 500 bp to 800 bp, from 500 bp to 700 bp, or from 500 bp to 600 bp, but is not limited thereto. It is clear that it may be appropriately determined or understood by those skilled in the relevant art.

The disclosed method comprises bringing into contact with a cell the Cas12f1 system, vector system, composition, or virus of the present disclosure.

In an embodiment, the bringing-into-contact with the cell may comprise delivering or introducing, into the cell, the Cas12f1 system, vector system, composition, or virus of the present disclosure.

The nucleic acid or nucleic acid construct (for example, a vector) of the present disclosure may be delivered or introduced, for example, by in vivo electroporation, liposomes, nanoparticles, or DNA injection or DNA vaccination, with or without a recombinant vector.

The vector system of the present disclosure may be delivered or introduced by a virus, such as a retrovirus, a lentivirus, an adenovirus, an adeno-associated virus, a vaccinia virus, a poxvirus, a herpes simplex virus, or a phage. Specifically, the vector system may be contained in a packaging virus and delivered into a cell in the form of a virus produced by the packaging virus.

Specifically, the bringing-into-contact, delivery, or introduction may be made by electroporation, gene gun, sonoporation, magnetofection, nanoparticles, and/or transient cell compression or squeezing method. When the cell is a eukaryotic cell, cationic liposome method, lithium acetate-DMSO, lipid-mediated transfection, calcium phosphate precipitation, lipofection, polyethyleneimine (PEI)-mediated transfection, DEAE-dextran-mediated transfection, and/or nanoparticle-mediated nucleic acid delivery (see Panyam et al., Adv Drug Deliv Rev. 2012 Sep. 13. pii: 50169-409X(12)00283-9) may be used.

In another embodiment, the bringing-into-contact, delivery, or introduction may be performed in vitro, in vivo, or ex vivo.

In an embodiment, the cell may be a plant cell, a non-human animal cell, or a human cell. In addition, the cell may be a eukaryotic cell or a prokaryotic cell. In addition, the cell may be a cell of a patient with muscular dystrophy.

Additionally, as described herein, there is provided a method for treating Duchenne muscular dystrophy comprising administering to a subject the Cas12f1 system, vector system, composition, or virus of the present disclosure.

In an embodiment, the subject may be a subject, such as mammal, including a human, having Duchenne muscular dystrophy.

In another embodiment, the Cas12f1 system, vector system, composition, or virus of the present disclosure may be administered to a muscle, such as skeletal muscle, cardiac muscle, or tibialis muscle, of the subject.

### EXEMPLARY EMBODIMENTS

### Embodiment 1

An editing system for a dystrophin gene, comprising:
an endonuclease comprising Cas12f1 or a variant protein thereof, or a nucleic acid encoding the endonuclease;
an engineered guide RNA comprising a first guide sequence that hybridizes to a target sequence in a dystrophin gene, or a nucleic acid encoding the guide RNA; and
an engineered guide RNA comprising a second guide sequence that hybridizes to a target sequence in a dystrophin gene, or a nucleic acid encoding the guide RNA,
wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and
the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

### Embodiment 2

A composition, comprising:
an endonuclease comprising Cas12f1 or a variant protein thereof, or a nucleic acid encoding the endonuclease;
an engineered guide RNA comprising a first guide sequence that hybridizes to a target sequence in a dystrophin gene, or a nucleic acid encoding the guide RNA; and
an engineered guide RNA comprising a second guide sequence that hybridizes to a target sequence in a dystrophin gene, or a nucleic acid encoding the guide RNA,
wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and
the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

### Embodiment 3

The system or composition of any one of the above-described embodiments,
wherein the system or composition is applied to a cell to cause deletion of dystrophin exon 51.

### Embodiment 4

The system or composition of any one of the above-described embodiments,
wherein the system or composition is for treatment of Duchenne muscular dystrophy.

### Embodiment 5

The system or composition of any one of the above-described embodiments,
wherein the first guide sequence is a sequence hybridizable to a target sequence that is complementary to a nucleotide sequence selected from the group consisting of SEQ ID NOs: 190 to 217 and SEQ ID NOs: 255 to 280, wherein the nucleotide sequence is located in a non-target strand of a region 5000 bp upstream of dystrophin exon 51, and
the second guide sequence is a sequence hybridizable to a target sequence that is complementary to a nucleotide sequence selected from the group consisting of SEQ ID NOs: 218 to 254 and SEQ ID NOs: 281 to 311, wherein the nucleotide sequence is located in a non-target strand of a region 5000 bp downstream of dystrophin exon 51.

### Embodiment 6

The system or composition of any one of the above-described embodiments,
wherein the first guide sequence comprises a sequence of contiguous 15 to 20 nucleotides from a nucleotide sequence selected from the group consisting of SEQ ID NOs: 190 to 217 and SEQ ID NOs: 255 to 280, wherein thymine (T) in the contiguous nucleotide sequence is substituted with uracil (U) and/or,
the second guide sequence comprises a sequence of contiguous 15 to 20 nucleotides from a nucleotide sequence selected from the group consisting of SEQ ID NOs: 218 to 254 and SEQ ID NOs: 281 to 311, wherein thymine (T) in the contiguous nucleotide sequence is substituted with uracil (U).

### Embodiment 7

The system or composition of any one of the above-described embodiments,
wherein the first guide sequence comprises a nucleotide sequence selected from the group consisting of SEQ ID NOs: 312 to 323 and/or,
the second guide sequence comprises a nucleotide sequence selected from the group consisting of SEQ ID NOs: 324 to 335.

### Embodiment 8

The system or composition of any one of the above-described embodiments,
wherein the engineered guide RNA comprises a U-rich tail sequence linked to the 3′-end of the first or second guide sequence, in which the U-rich tail is represented by 5′-(UₘV)ₙUₒ-3′, wherein V is each independently A, C, or G, m and o are integers between 1 and 20, and n is an integer between 0 and 5.

### Embodiment 9

The system or composition of any one of the above-described embodiments,
wherein the engineered guide RNA comprises a nucleotide sequence having at least 50% sequence identity to a scaffold region of a wild-type Cas12f1 guide RNA sequence, in which the scaffold region of the wild-type Cas12f1 guide RNA sequence sequentially comprises, from the 5′-end, a first stem-loop region, a second stem-loop region, a third stem-loop region, a fourth stem-loop region, and a tracrRNA-crRNA complementarity region, and
the engineered guide RNA comprises at least one modification selected from the group consisting of the following (1) to (5) with respect to the wild-type Cas12f1 guide RNA sequence:
(1) deletion of at least a part of the first stem-loop region;
(2) deletion of at least a part of the second stem-loop region;
(3) deletion of at least a part of the tracrRNA-crRNA complementarity region;
(4) replacement of one or more uracil (U) residues with A, G, or C in three or more consecutive U residues when the consecutive U residues are present in the tracrRNA-crRNA complementarity region; and
(5) addition of a U-rich tail to the 3′-end of the crRNA sequence (in which a sequence of the U-rich tail is represented by 5′-(UₘV)ₙUₒ-3′, wherein V is each independently A, C, or G, m and o are integers between 1 and 20, and n is an integer between 0 and 5).

### Embodiment 10

The system or composition of any one of the above-described embodiments,
wherein the wild-type Cas12f1 guide RNA comprises tracrRNA comprising the nucleotide sequence of SEQ ID NO: 11 and crRNA comprising the nucleotide sequence of SEQ ID NO: 12.

### Embodiment 11

The system or composition of any one of the above-described embodiments,
wherein the engineered guide RNA comprises at least one modification selected from (5) addition of a U-rich tail to the 3′-end of the crRNA sequence and (4) replacement of one or more uracil (U) residues with A, G, or C in three or more consecutive U residues when the consecutive U residues are present in the tracrRNA-crRNA complementarity region.

### Embodiment 12

The system or composition of any one of the above-described embodiments,
wherein the engineered guide RNA comprises at least one modification selected from (1) deletion of at least a part of the first stem-loop region; (2) deletion of at least a part of the second stem-loop region; and (3) deletion of at least a part of the tracrRNA-crRNA complementarity region.

### Embodiment 13

The system or composition of any one of the above-described embodiments,
wherein the engineered guide RNA comprises (3) deletion of a part of the tracrRNA-crRNA complementarity region, wherein the part of the complementarity region consists of 1 to 54 nucleotides.

### Embodiment 14

The system or composition of any one of the above-described embodiments,
wherein the engineered guide RNA comprises (3) deletion of the entire tracrRNA-crRNA complementarity region, wherein the entire complementarity region consists of 55 nucleotides.

### Embodiment 15

The system or composition of any one of the above-described embodiments,
wherein the engineered guide RNA comprises (1) deletion of at least a part of the first stem-loop region, wherein the at least a part of the stem-loop region consists of 1 to 20 nucleotides.

### Embodiment 16

The system or composition of any one of the above-described embodiments,
wherein the engineered guide RNA comprises (2) deletion of at least a part of the second stem-loop region, wherein the at least a part of the stem-loop region consists of 1 to 27 nucleotides.

### Embodiment 17

The system or composition of any one of the above-described embodiments,
wherein the engineered guide RNA comprises at least one modification selected from (1) deletion of at least a part of the first stem-loop region; and (5) addition of a U-rich tail to the 3′-end of the crRNA sequence.

### Embodiment 18

The system or composition of any one of the above-described embodiments,
wherein the engineered guide RNA consists of a sequence represented by following Formula (I) or has at least 80% sequence identity to the sequence:

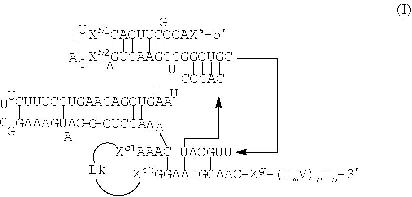

in Formula (I),
X^{a}, X^{b1}, X^{b2}, X^{c1}, and X^{c2} each independently consist of 0 to 35 (poly)nucleotides,
X^{g} is the first or second guide sequence,
Lk is a polynucleotide linker of 2 to 20 nucleotides in length or is absent, and
(UₘV)ₙUₒ is present as a U-rich or absent, and in a case where the U-rich tail is present, U is uridine, V is each independently A, C or G, m and o are each independently an integer between 1 and 20, and n is an integer between 0 and 5.

### Embodiment 19

The system or composition of any one of the above-described embodiments,
wherein X^{a} comprises the nucleotide sequence of SEQ ID NO: 14 or a deleted form of the sequence of SEQ ID NO: 14 with 1 to 20 nucleotides deleted therefrom.

### Embodiment 20

The system or composition of any one of the above-described embodiments,
wherein X^{b1} comprises the nucleotide sequence of SEQ ID NO: 25 or a deleted form of the sequence of SEQ ID NO: 25 with 1 to 13 nucleotides deleted therefrom.

### Embodiment 21

The system or composition of any one of the above-described embodiments,
wherein X^{b2} comprises the nucleotide sequence of SEQ ID NO: 29 or a deleted form of the sequence of SEQ ID NO: 29 with 1 to 14 nucleotides deleted therefrom.

### Embodiment 22

The system or composition of any one of the above-described embodiments,
wherein the sequence 5′-X^{b1}UUAGX^{b2}-3′ in Formula (I) is a nucleotide sequence selected from the group consisting of SEQ ID NOs: 34 to 38.

### Embodiment 23

The system or composition of any one of the above-described embodiments,
wherein X^{c1} comprises the nucleotide sequence of SEQ ID NO: 39 or a deleted form of the sequence of SEQ ID NO: 39 with 1 to 28 nucleotides deleted therefrom.

### Embodiment 24

The system or composition of any one of the above-described embodiments,
wherein in a case where three or more consecutive uracil (U) residues are present in a sequence of X^{c1}, the sequence of X^{c1} comprises a modification in which at least one U residue thereof is replaced with A, G or C.

### Embodiment 25

The system or composition of any one of the above-described embodiments,
wherein X^{c2} comprises the nucleotide sequence of SEQ ID NO: 58 or a deleted form of the sequence of SEQ ID NO: 58 with 1 to 27 nucleotides deleted therefrom.

### Embodiment 26

The system or composition of any one of the above-described embodiments,
wherein in a case where the sequence 5′-ACGAA-3′ is present in X^{c2}, the sequence is replaced with 5′-NGNNN-3′, wherein N is each independently A, C, G, or U.

### Embodiment 27

The system or composition of any one of the above-described embodiments,
wherein the sequence 5′-X^{c1}-Lk-X^{c2}-3′ in Formula (I) is a nucleotide sequence selected from the group consisting of SEQ ID NOs: 80 to 86.

### Embodiment 28

The system or composition of any one of the above-described embodiments,
wherein Lk comprises a nucleotide sequence selected from the group consisting of 5′-GAAA-3′, 5′-UUAG-3′, 5′-UGAAAA-3′, 5′-UUGAAAAA-3′, 5′-UUCGAAAGAA-3′ (SEQ ID NO: 76), 5′-UUCAGAAAUGAA-3′ (SEQ ID NO: 77), 5′-UUCAUGAAAAUGAA-3′ (SEQ ID NO: 78), and 5′-UUCAUUGAAAAAUGAA-3′ (SEQ ID NO: 79).

### Embodiment 29

The system or composition of any one of the above-described embodiments,
wherein (UₘV)ₙUₒ is such that (i) n is 0 and o is an integer between 1 and 6, or (ii) V is A or G, m and o are each independently an integer between 3 and 6, and n is an integer between 1 and 3.

### Embodiment 30

The system or composition of any one of the above-described embodiments,
wherein the engineered guide RNA comprises an engineered tracrRNA consisting of a nucleotide sequence selected from the group consisting of SEQ ID NOs: 87 to 132.

### Embodiment 31

The system or composition of any one of the above-described embodiments,
wherein the engineered guide RNA comprises an engineered crRNA, wherein the engineered crRNA comprises a nucleotide sequence selected from the group consisting of SEQ ID NOs: 133 to 148.

### Embodiment 32

The system or composition of any one of the above-described embodiments,
wherein the engineered guide RNA is a dual guide RNA or a single guide RNA.

### Embodiment 33

The system or composition of any one of the above-described embodiments,
wherein the engineered single guide RNA consists of a nucleotide sequence selected from the group consisting of SEQ ID NOs: 149 to 186.

### Embodiment 34

The system or composition of any one of the above-described embodiments,
wherein the Cas12f1 or variant protein thereof induces a double-strand break in or outside the target sequence.

### Embodiment 35

The system or composition of any one of the above-described embodiments,
wherein the Cas12f1 or variant protein thereof comprises an amino acid sequence having at least 70% sequence identity to an amino acid sequence selected from the group consisting of SEQ ID NOs: 1 to 5.

### Embodiment 36

The system or composition of any one of the above-described embodiments,
wherein the Cas12f1 or variant protein thereof has at least 70% sequence identity to the amino acid sequence of SEQ ID NO: 1 or 5.

### Embodiment 37

The system or composition of any one of the above-described embodiments,
wherein the endonuclease comprising Cas12f1 or a variant protein thereof, and the engineered guide RNA comprising a first guide sequence or the engineered guide RNA comprising a second guide sequence are in the form of a ribonucleoprotein (RNP).

### Embodiment 38

The system or composition of any one of the above-described embodiments,
wherein the system or composition further comprises a molecule that inhibits expression of a gene involved in non-homologous end joining (NHEJ) or a nucleic acid encoding the molecule.

### Embodiment 39

The system or composition of any one of the above-described embodiments,
wherein the gene involved in NHEJ is at least one selected from the group consisting of ATM1, XRCC4, XLF, XRCC6, LIG4, and DCLRE1C.

### Embodiment 40

The system or composition of any one of the above-described embodiments,
wherein the gene involved in NHEJ is at least one selected from the group consisting of XRCC6 and DCLRE1C.

### Embodiment 41

The system or composition of any one of the above-described embodiments,
wherein the molecule is shRNA, siRNA, miRNA, or an antisense oligonucleotide.

### Embodiment 42

The system or composition of any one of the above-described embodiments,
wherein the shRNA molecule is at least one selected from the group consisting of shXRCC6 and shDCLRE1C.

### Embodiment 43

The system or composition of any one of the above-described embodiments,
wherein the shRNA molecule is at least one selected from the group consisting of SEQ ID NOs: 360 to 389 and 403.

### Embodiment 44

A vector system, comprising at least one vector that comprises:
a first nucleic acid construct to which a nucleotide sequence encoding an endonuclease is operably linked, the endonuclease comprising Cas12f1 or a variant protein thereof;
a second nucleic acid construct to which an engineered guide RNA or a nucleotide sequence encoding the engineered guide RNA is operably linked, the engineered guide RNA comprising a first guide sequence that hybridizes to a target sequence in a dystrophin gene; and
a third nucleic acid construct to which an engineered guide RNA or a nucleotide sequence encoding the engineered guide RNA is operably linked, the engineered guide RNA comprising a second guide sequence that hybridizes to a target sequence in a dystrophin gene,
wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and
the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

### Embodiment 45

The vector system of any one of the above-described embodiments,
wherein the vector system is for treatment of Duchenne muscular dystrophy.

### Embodiment 46

The vector system of any one of the above-described embodiments,
wherein the first guide sequence is a sequence hybridizable to a target sequence that corresponds to a nucleotide sequence selected from the group consisting of SEQ ID NOs: 190 to 217 and SEQ ID NOs: 255 to 280, wherein the nucleotide sequence is located in a non-target strand of a region 5000 bp upstream of dystrophin exon 51, and
the second guide sequence is a sequence hybridizable to a target sequence that corresponds to a nucleotide sequence selected from the group consisting of SEQ ID NOs: 218 to 254 and SEQ ID NOs: 281 to 311, wherein the nucleotide sequence is located in a non-target strand of a region 5000 bp downstream of dystrophin exon 51.

### Embodiment 47

The vector system of any one of the above-described embodiments,
wherein the first guide sequence consists of a sequence of contiguous 15 to 20 nucleotides from a nucleotide sequence selected from the group consisting of SEQ ID NOs: 190 to 217 and SEQ ID NOs: 255 to 280, wherein thymine (T) in the contiguous nucleotide sequence is substituted with uracil (U) and/or,
the second guide sequence consists of a sequence of contiguous 15 to 20 nucleotides from a nucleotide sequence selected from the group consisting of SEQ ID NOs: 218 to 254 and SEQ ID NOs: 281 to 311, wherein thymine (T) in the contiguous nucleotide sequence is substituted with uracil (U).

### Embodiment 48

The vector system of any one of the above-described embodiments,
wherein the first guide sequence is a nucleotide sequence selected from the group consisting of SEQ ID NOs: 312 to 323 and/or,
the second guide sequence is a nucleotide sequence selected from the group consisting of SEQ ID NOs: 324 to 335.

### Embodiment 49

The vector system of any one of the above-described embodiments,
wherein the engineered guide RNA comprises a U-rich tail sequence linked to the 3′-end of the first or second guide sequence, in which the U-rich tail is represented by 5′-(UₘV)ₙUₒ-3′, wherein V is each independently A, C, or G, m and o are integers between 1 and 20, and n is an integer between 0 and 5.

### Embodiment 50

The vector system of any one of the above-described embodiments,
wherein the engineered guide RNA comprises a nucleotide sequence having at least 50% sequence identity to a wild-type Cas12f1 guide RNA sequence, in which the engineered guide RNA sequentially comprises, from the 5′-end, a first stem-loop region, a second stem-loop region, a third stem-loop region, a fourth stem-loop region, a tracrRNA-crRNA complementarity region, and a guide sequence, and
the engineered guide RNA comprises at least one modification selected from the group consisting of the following (1) to (5) with respect to the wild-type Cas12f1 guide RNA sequence:
(1) deletion of at least a part of the first stem-loop region;
(2) deletion of at least a part of the second stem-loop region;
(3) deletion of at least a part of the tracrRNA-crRNA complementarity region;
(4) replacement of one or more uracil (U) residues with A, G, or C in three or more consecutive U residues when the consecutive U residues are present in the tracrRNA-crRNA complementarity region; and
(5) addition of a U-rich tail to the 3′-end of the crRNA sequence (in which a sequence of the U-rich tail is represented by 5′-(UₘV)ₙUₒ-3′, wherein V is each independently A, C, or G, m and o are integers between 1 and 20, and n is an integer between 0 and 5).

### Embodiment 51

The vector system of any one of the above-described embodiments,
wherein the wild-type Cas12f1 guide RNA comprises tracrRNA comprising the nucleotide sequence of SEQ ID NO: 11 and crRNA comprising the nucleotide sequence of SEQ ID NO: 12.

### Embodiment 52

The vector system of any one of the above-described embodiments,
wherein the engineered guide RNA comprises at least one modification selected from (5) addition of a U-rich tail to the 3′-end of the crRNA sequence and (4) replacement of one or more uracil (U) residues with A, G, or C in three or more consecutive U residues when the consecutive U residues are present in the tracrRNA-crRNA complementarity region.

### Embodiment 53

The vector system of any one of the above-described embodiments,
wherein the engineered guide RNA comprises at least one modification selected from the group consisting of (1) deletion of at least a part of the first stem-loop region; (2) deletion of at least a part of the second stem-loop region; and (3) deletion of at least a part of the tracrRNA-crRNA complementarity region.

### Embodiment 54

The vector system of any one of the above-described embodiments,
wherein the engineered guide RNA comprises (3) deletion of a part of the tracrRNA-crRNA complementarity region, wherein the part of the complementarity region consists of 1 to 54 nucleotides.

### Embodiment 55

The vector system of any one of the above-described embodiments,
wherein the engineered guide RNA comprises (3) deletion of the entire tracrRNA-crRNA complementarity region, wherein the entire complementarity region consists of 55 nucleotides.

### Embodiment 56

The vector system of any one of the above-described embodiments,
wherein the engineered guide RNA comprises (1) deletion of at least a part of the first stem-loop region, wherein the at least a part of the stem-loop region consists of 1 to 20 nucleotides.

### Embodiment 57

The vector system of any one of the above-described embodiments,
wherein the engineered guide RNA comprises (2) deletion of at least a part of the second stem-loop region, wherein the at least a part of the stem-loop region consists of 1 to 27 nucleotides.

### Embodiment 58

The vector system of any one of the above-described embodiments,
wherein the engineered guide RNA comprises at least one modification selected from (1) deletion of at least a part of the first stem-loop region; and (5) addition of a U-rich tail to the 3′-end of the crRNA sequence.

### Embodiment 59

The vector system of any one of the above-described embodiments,
wherein the engineered guide RNA consists of a sequence represented by following Formula (I) or has at least 80% sequence identity to the sequence:

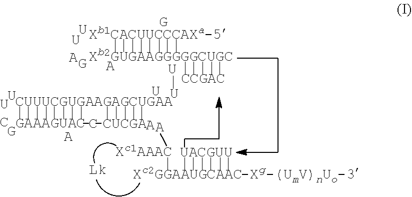

in Formula (I),
X^{a}, X^{b1}, X^{b2}, X^{c1}, and X^{c2} each independently consist of 0 to 35 (poly)nucleotides,
X^{g} is the first or second guide sequence,
Lk is a polynucleotide linker of 2 to 20 nucleotides in length or is absent, and
(UₘV)ₙUₒ is a U-rich tail and is present or absent, and in a case where the U-rich tail is present, U is uridine, V is each independently A, C or G, m and o are each independently an integer between 1 and 20, and n is an integer between 0 and 5.

### Embodiment 60

The vector system of any one of the above-described embodiments,
wherein X^{a} comprises the nucleotide sequence of SEQ ID NO: 14 or a deleted form of the sequence of SEQ ID NO: 14 with 1 to 20 nucleotides deleted therefrom.

### Embodiment 61

The vector system of any one of the above-described embodiments,
wherein X^{b1} comprises the nucleotide sequence of SEQ ID NO: 25 or a deleted form of the sequence of SEQ ID NO: 25 with 1 to 13 nucleotides deleted therefrom.

### Embodiment 62

The vector system of any one of the above-described embodiments,
wherein X^{b2} comprises the nucleotide sequence of SEQ ID NO: 29 or a deleted form of the sequence of SEQ ID NO: 29 with 1 to 14 nucleotides deleted therefrom.

### Embodiment 63

The vector system of any one of the above-described embodiments,
wherein the sequence 5′-X^{b1}UUAGX^{b2}-3′ in Formula (I) is a nucleotide sequence selected from the group consisting of SEQ ID NOs: 34 to 38.

### Embodiment 64

The vector system of any one of the above-described embodiments,
wherein X^{c1} comprises the nucleotide sequence of SEQ ID NO: 39 or a deleted form of the sequence of SEQ ID NO: 39 with 1 to 28 nucleotides deleted therefrom.

### Embodiment 65

The vector system of any one of the above-described embodiments,
wherein in a case where three or more consecutive uracil (U) residues are present in a sequence of X^{c1}, the sequence of X^{c1} comprises a modification in which at least one U residue thereof is replaced with A, G or C.

### Embodiment 66

The vector system of any one of the above-described embodiments,
wherein X^{c2} comprises the nucleotide sequence of SEQ ID NO: 58 or a deleted form of the sequence of SEQ ID NO: 58 with 1 to 27 nucleotides deleted therefrom.

### Embodiment 67

The vector system of any one of the above-described embodiments,
wherein in a case where the sequence 5′-ACGAA-3′ is present in X^{c2}, the sequence is replaced with 5′-NGNNN-3′, wherein N is each independently A, C, G or U.

### Embodiment 68

The vector system of any one of the above-described embodiments,
wherein the sequence 5′-X^{c1}-Lk-X^{c2}-3′ in Formula (I) is a nucleotide sequence selected from the group consisting of SEQ ID NOs: 80 to 86.

### Embodiment 69

The vector system of any one of the above-described embodiments,
wherein Lk comprises a nucleotide sequence selected from the group consisting of 5′-GAAA-3′, 5′-UUAG-3′, 5′-UGAAAA-3′, 5′-UUGAAAAA-3′, 5′-UUCGAAAGAA-3′ (SEQ ID NO: 76), 5′-UUCAGAAAUGAA-3′ (SEQ ID NO: 77), 5′-UUCAUGAAAAUGAA-3′ (SEQ ID NO: 78), and 5′-UUCAUUGAAAAAUGAA-3′ (SEQ ID NO: 79).

### Embodiment 70

The vector system of any one of the above-described embodiments,
wherein (UₘV)ₙUₒ is such that (i) n is 0 and o is an integer between 1 and 6, or (ii) V is A or G, m and o are each independently an integer between 3 and 6, and n is an integer between 1 and 3.

### Embodiment 71

The vector system of any one of the above-described embodiments,
wherein the engineered guide RNA comprises an engineered tracrRNA consisting of a nucleotide sequence selected from the group consisting of SEQ ID NOs: 87 to 132.

### Embodiment 72

The vector system of any one of the above-described embodiments,
wherein the engineered guide RNA comprises an engineered crRNA, wherein the engineered crRNA comprises a nucleotide sequence selected from the group consisting of SEQ ID NOs: 133 to 148.

### Embodiment 73

The vector system of any one of the above-described embodiments,
wherein the engineered guide RNA is a dual guide RNA or a single guide RNA.

### Embodiment 74

The vector system of any one of the above-described embodiments,
wherein the engineered single guide RNA consists of a nucleotide sequence selected from the group consisting of SEQ ID NOs: 149 to 186.

### Embodiment 75

The vector system of any one of the above-described embodiments,
wherein the Cas12f1 or variant protein thereof comprises an amino acid sequence having at least 70% sequence identity to an amino acid sequence selected from the group consisting of SEQ ID NOs: 1 to 5.

### Embodiment 76

The vector system of any one of the above-described embodiments,
wherein the Cas12f1 or variant protein thereof has at least 70% sequence identity to the amino acid sequence of SEQ ID NO: 1 or 5.

### Embodiment 77

The vector system of any one of the above-described embodiments,
wherein the endonuclease comprising Cas12f1 or a variant protein thereof, and the engineered guide RNA comprising a first guide sequence or the engineered guide RNA comprising a second guide sequence are in the form of a ribonucleoprotein (RNP).

### Embodiment 78

The vector system of any one of the above-described embodiments,
wherein the system further comprises a molecule that inhibits expression of a gene involved in non-homologous end joining (NHEJ) or a nucleic acid encoding the molecule.

### Embodiment 79

The vector system of any one of the above-described embodiments,
wherein the gene involved in NHEJ is at least one selected from the group consisting of ATM1, XRCC4, XLF, XRCC6, LIG4, and DCLRE1C.

### Embodiment 80

The vector system of any one of the above-described embodiments,
wherein the gene involved in NHEJ is at least one selected from the group consisting of XRCC6 and DCLRE1C.

### Embodiment 81

The vector system of any one of the above-described embodiments,
wherein the molecule is shRNA, siRNA, miRNA, or an antisense oligonucleotide.

### Embodiment 82

The vector system of any one of the above-described embodiments,
wherein the nucleic acid constructs included in the vector system are located in same or different vectors.

### Embodiment 83

The vector system of any one of the above-described embodiments,
wherein respective components in the vector are included in one vector.

### Embodiment 84

The vector system of any one of the above-described embodiments,
wherein the vector further comprises a promoter or an enhancer.

### Embodiment 85

The vector system of any one of the above-described embodiments,
wherein the promoter is U6 promoter, EFS promoter, EF1-α promoter, H1 promoter, 7SK promoter, CMV promoter, LTR promoter, Ad MLP promoter, HSV promoter, SV40 promoter, CBA promoter, or RSV promoter.

### Embodiment 86

The vector system of any one of the above-described embodiments,
wherein the vector is at least one viral vector selected from the group consisting of a retroviral (retrovirus) vector, a lentiviral (lentivirus) vector, an adenoviral (adenovirus) vector, an adeno-associated viral (adeno-associated virus) vector, a vaccinia viral (vaccinia virus) vector, a poxviral (poxvirus) vector, a herpes simplex viral (herpes simplex virus) vector, and a phagemid vector.

### Embodiment 87

The vector system of any one of the above-described embodiments,
wherein the vector is an adeno-associated viral vector, and the adeno-associated viral vector is capable of comprising, in a single vector, all components within the vector.

### Embodiment 88

The vector system of any one of the above-described embodiments,
wherein the vector is at least one non-viral vector selected from the group consisting of plasmid, naked DNA, DNA complex, mRNA (transcript), and amplicon.

### Embodiment 89

The vector system of any one of the above-described embodiments,
wherein the plasmid is at least one selected from the group consisting of pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, and pUC19.

### Embodiment 90

An engineered guide RNA, comprising a spacer region, which comprises a guide sequence capable of hybridizing to a target sequence in a dystrophin gene, and a scaffold region,
wherein the guide sequence is capable of hybridizing to a target sequence of contiguous 15 to 30 bp in length, wherein the target sequence is adjacent to the 5′-end or the 3′-end of a protospacer-adjacent motif (PAM) sequence which is present in a region 5000 bp upstream or downstream of dystrophin exon 51 and is recognized by Cas12f1 or a variant protein thereof.

### Embodiment 91

The engineered guide RNA of any one of the above-described embodiments,
wherein the PAM sequence is 5′-TTTA-3′ or 5′-TTTG-3′.

### Embodiment 92

The engineered guide RNA of any one of the above-described embodiments,
wherein the guide sequence comprises a sequence capable of hybridizing to a target sequence complementary to a nucleotide sequence selected from the group consisting of SEQ ID NOs: 190 to 311 present in a non-target strand of a region 5000 bp upstream or downstream of dystrophin exon 51.

### Embodiment 93

The engineered guide RNA of any one of the above-described embodiments,
wherein the guide sequence comprises a sequence of contiguous 15 to 20 nucleotides from a nucleotide sequence selected from the group consisting of SEQ ID NOs: 190 to 311, wherein thymine (T) in the contiguous nucleotide sequence is substituted with uracil (U).

### Embodiment 94

The engineered guide RNA of any one of the above-described embodiments,
wherein the guide sequence comprises a nucleotide sequence selected from the group consisting of SEQ ID NOs: 311 to 335.

### Embodiment 95

The engineered guide RNA of any one of the above-described embodiments,
wherein the engineered guide RNA comprises a U-rich tail sequence linked to the 3′-end of the guide sequence, in which the U-rich tail is represented by 5′-(UₘV)ₙUₒ-3′, wherein V is each independently A, C, or G, m and o are integers between 1 and 20, and n is an integer between 0 and 5.

### Embodiment 96

The engineered guide RNA of any one of the above-described embodiments,
wherein the scaffold region comprises a nucleotide sequence having at least 50% sequence identity to a scaffold region of a wild-type Cas12f1 guide RNA sequence, in which the scaffold region of the wild-type Cas12f1 guide RNA sequence sequentially comprises, from the 5′-end, a first stem-loop region, a second stem-loop region, a third stem-loop region, a fourth stem-loop region, and a tracrRNA-crRNA complementarity region, and
the engineered guide RNA comprises at least one modification selected from the group consisting of the following (1) to (5) with respect to the wild-type Cas12f1 guide RNA sequence:
(1) deletion of at least a part of the first stem-loop region;
(2) deletion of at least a part of the second stem-loop region;
(3) deletion of at least a part of the tracrRNA-crRNA complementarity region;
(4) replacement of one or more uracil (U) residues with A, G, or C in three or more consecutive U residues when the consecutive U residues are present in the tracrRNA-crRNA complementarity region; and
(5) addition of a U-rich tail to the 3′-end of the crRNA sequence (in which a sequence of the U-rich tail is represented by 5′-(UₘV)ₙUₒ-3′, wherein V is each independently A, C, or G, m and o are integers between 1 and 20, and n is an integer between 0 and 5).

### Embodiment 97

The engineered guide RNA of any one of the above-described embodiments,
wherein the wild-type Cas12f1 guide RNA comprises tracrRNA comprising the nucleotide sequence of SEQ ID NO: 11 and crRNA comprising the nucleotide sequence of SEQ ID NO: 12.

### Embodiment 98

The engineered guide RNA of any one of the above-described embodiments,
wherein the engineered guide RNA comprises at least one modification selected from (5) addition of a U-rich tail to the 3′-end of the crRNA sequence and (4) replacement of one or more uracil (U) residues with A, G, or C in three or more consecutive U residues when the consecutive U residues are present in the tracrRNA-crRNA complementarity region.

### Embodiment 99

The engineered guide RNA of any one of the above-described embodiments,
wherein the engineered guide RNA comprises at least one modification selected from (1) deletion of at least a part of the first stem-loop region; (2) deletion of at least a part of the second stem-loop region; and (3) deletion of at least a part of the tracrRNA-crRNA complementarity region.

### Embodiment 100

The engineered guide RNA of any one of the above-described embodiments,
wherein the engineered guide RNA comprises (3) deletion of a part of the tracrRNA-crRNA complementarity region, wherein the part of the complementarity region consists of 1 to 54 nucleotides.

### Embodiment 101

The engineered guide RNA of any one of the above-described embodiments,
wherein the engineered guide RNA comprises (3) deletion of the entire tracrRNA-crRNA complementarity region, wherein the entire complementarity region consists of 55 nucleotides.

### Embodiment 102

The engineered guide RNA of any one of the above-described embodiments,
wherein the engineered guide RNA comprises (1) deletion of at least a part of the first stem-loop region, wherein the at least a part of the stem-loop region consists of 1 to 20 nucleotides.

### Embodiment 103

The engineered guide RNA of any one of the above-described embodiments,
wherein the engineered guide RNA comprises (2) deletion of at least a part of the second stem-loop region, wherein the at least a part of the stem-loop region consists of 1 to 27 nucleotides.

### Embodiment 104

The engineered guide RNA of any one of the above-described embodiments,
wherein the engineered guide RNA comprises at least one modification selected from (1) deletion of at least a part of the first stem-loop region; and (5) addition of a U-rich tail to the 3′-end of the crRNA sequence.

### Embodiment 105

The engineered guide RNA of any one of the above-described embodiments,
wherein the engineered guide RNA is represented by following Formula (I):

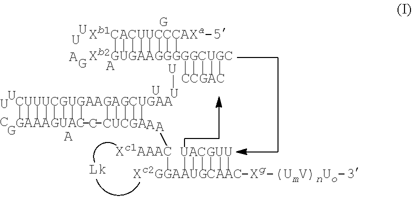

in Formula (I),
X^{a}, X^{b1}, X^{b2}, X^{c1}, and X^{c2} each independently consist of 0 to 35 (poly)nucleotides,
X^{g} is a guide sequence,
Lk is a polynucleotide linker of 2 to 20 nucleotides in length or is absent, and
(UₘV)ₙUₒ is present as a U-rich or absent, and in a case where the U-rich tail is present, U is uridine, V is each independently A, C or G, m and o are each independently an integer between 1 and 20, and n is an integer between 0 and 5.

### Embodiment 106

The engineered guide RNA of any one of the above-described embodiments,
wherein X^{a} comprises the nucleotide sequence of SEQ ID NO: 14 or a deleted form of the sequence of SEQ ID NO: 14 with 1 to 20 nucleotides deleted therefrom.

### Embodiment 107

The engineered guide RNA of any one of the above-described embodiments,
wherein X^{b1} comprises the nucleotide sequence of SEQ ID NO: 25 or a deleted form of the sequence of SEQ ID NO: 25 with 1 to 13 nucleotides deleted therefrom.

### Embodiment 108

The engineered guide RNA of any one of the above-described embodiments,
wherein X^{b2} comprises the nucleotide sequence of SEQ ID NO: 29 or a deleted form of the sequence of SEQ ID NO: 29 with 1 to 14 nucleotides deleted therefrom.

### Embodiment 109

The engineered guide RNA of any one of the above-described embodiments,
wherein the sequence 5′-X^{b1}UUAGX^{b2}-3′ in Formula (I) is a nucleotide sequence selected from the group consisting of SEQ ID NOs: 34 to 38.

### Embodiment 110

The engineered guide RNA of any one of the above-described embodiments,
wherein X^{c1} comprises the nucleotide sequence of SEQ ID NO: 39 or a deleted form of the sequence of SEQ ID NO: 39 with 1 to 28 nucleotides deleted therefrom.

### Embodiment 111

The engineered guide RNA of any one of the above-described embodiments,
wherein in a case where three or more consecutive uracil (U) residues are present in a sequence of X^{c1}, the sequence of X^{c1} sequence comprises a modification in which at least one U residue thereof is replaced with A, G or C.

### Embodiment 112

The engineered guide RNA of any one of the above-described embodiments,
wherein X^{c2} comprises the nucleotide sequence of SEQ ID NO: 58 or a deleted form of the sequence of SEQ ID NO: 58 with 1 to 27 nucleotides deleted therefrom.

### Embodiment 113

The engineered guide RNA of any one of the above-described embodiments,
wherein in a case where the sequence 5′-ACGAA-3′ is present in X^{c2}, the sequence is replaced with 5′-NGNNN-3′, wherein N is each independently A, C, G or U.

### Embodiment 114

The engineered guide RNA of any one of the above-described embodiments,
wherein the sequence 5′-X^{c1}-Lk-X^{c2}-3′ in Formula (I) is a nucleotide sequence selected from the group consisting of SEQ ID NOs: 80 to 86.

### Embodiment 115

The engineered guide RNA of any one of the above-described embodiments,
wherein Lk comprises a nucleotide sequence selected from the group consisting of 5′-GAAA-3′, 5′-UUAG-3′, 5′-UGAAAA-3′, 5′-UUGAAAAA-3′, 5′-UUCGAAAGAA-3′ (SEQ ID NO: 76), 5′-UUCAGAAAUGAA-3′ (SEQ ID NO: 77), 5′-UUCAUGAAAAUGAA-3′ (SEQ ID NO: 78), and 5′-UUCAUUGAAAAAUGAA-3′ (SEQ ID NO: 79).

### Embodiment 116

The engineered guide RNA of any one of the above-described embodiments,
wherein (UₘV)ₙUₒ is such that (i) n is 0 and o is an integer between 1 and 6, or (ii) V is A or G, m and o are each independently an integer between 3 and 6, and n is an integer between 1 and 3.

### Embodiment 117

The engineered guide RNA of any one of the above-described embodiments,
wherein the engineered sgRNA comprises an engineered tracrRNA consisting of a nucleotide sequence selected from the group consisting of SEQ ID NOs: 87 to 132.

### Embodiment 118

The engineered guide RNA of any one of the above-described embodiments,
wherein the engineered guide RNA comprises an engineered crRNA, wherein the engineered crRNA comprises a nucleotide sequence selected from the group consisting of SEQ ID NOs: 133 to 148.

### Embodiment 119

The engineered guide RNA of any one of the above-described embodiments,
wherein the engineered guide RNA consists of a nucleotide sequence selected from the group consisting of SEQ ID NOs: 149 to 186.

### Embodiment 120

The engineered guide RNA of any one of the above-described embodiments,
wherein the engineered guide RNA is a sgRNA.

### Embodiment 121

A nucleic acid encoding the engineered guide RNA of any one of the above-described embodiments.

### Embodiment 122

A virus produced by the vector system of any one of the above-described embodiments.

### Embodiment 123

The virus of any one of the above-described embodiments,
wherein the virus is selected from the group consisting of retrovirus, lentivirus, adenovirus, adeno-associated virus, vaccinia virus, poxvirus, herpes simplex virus, and phage.

### Embodiment 124

A composition comprising the virus of any one of the above-described embodiments.

### Embodiment 125

A method for deleting a segment comprising exon 51 in a dystrophin gene in a cell,
comprising bringing into contact with the cell the system, composition, guide RNA, or vector system of any one of the above-described embodiments.

### Embodiment 126

The method of any one of the above-described embodiments,
wherein the cell is a prokaryotic or eukaryotic cell.

### Embodiment 127

The method of any one of the above-described embodiments,
wherein the eukaryotic cell is a yeast, an insect cell, a plant cell, a non-human animal cell, or a human cell.

### Embodiment 128

The method of any one of the above-described embodiments,
wherein the vector system is delivered into a prokaryotic or eukaryotic cell by electroporation, gene gun, sonication, magnetofection, transient cell compression or squeezing, cationic liposome, lithium acetate-DMSO, lipid-mediated transfection, calcium phosphate precipitation, lipofectamine, PEI (polyethyleneimine)-mediated transfection, DEAE-dextran-mediated transfection, or nanoparticle-mediated nucleic acid delivery.

### Embodiment 129

The method of any one of the above-described embodiments,
wherein the vector system is delivered directly into a prokaryotic cell or eukaryotic cell through at least one lipid nanoparticle (LNP).

### Embodiment 130

The method of any one of the above-described embodiments,
wherein the bringing-into-contact occurs in vivo or ex vivo.

### Form for Carrying Out the Disclosure

Hereinafter, the present disclosure will be described in more detail by the following examples. However, these examples are only intended to illustrate the present disclosure, and the scope of the present disclosure is not limited to these examples.

### Example 1. Construction of Nucleic Acid Editing System for Deletion of Dystrophin Exon 51

### Example 1.1. Production of Engineered gRNA

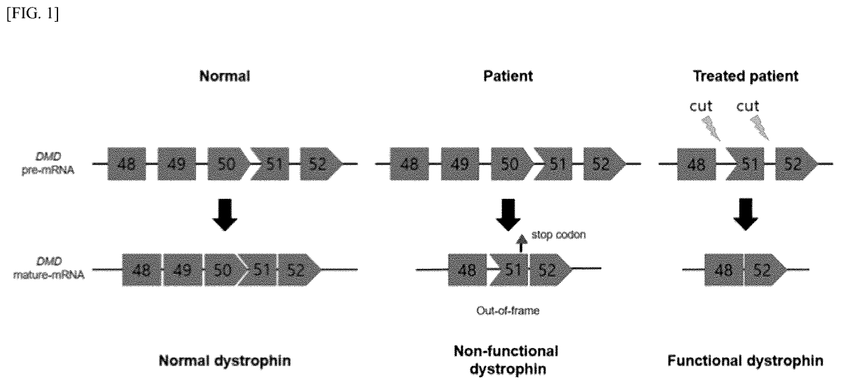

The most common type among patients with Duchenne muscular dystrophy (DMD) is a type in which a stop codon occurs in dystrophin exon 51. Referring to FIG. 1, deletion of exons 49 and 50 causes a stop codon, which serves as a signal to stop protein synthesis, to occur in exon 51, thereby preventing production of dystrophin protein. Here, deletion of exon 51 prevents production of the stop codon, which allows for production of a dystrophin protein that is shorter than normal and has normal function.

The CRISPR/Cas12f1 system and the TaRGET system for deletion of dystrophin exon 51 were constructed. In the systems, for the gRNAs having a guide sequence that hybridizes to a target sequence for deletion of exon 51, engineered gRNAs having at least one of the five modification sites (MS1, MS2, MS3, MS4, and MS5) as shown in FIG. 2 were produced, and the specific sequences thereof are shown in Table 4.

TABLE 4

| | | SEQ ID |
|---|---|---|
| gRNA | Sequence (5′ to 3′) | NO |
| Canonical | CUUCACUGAUAAAGUGGAGAACCGCUUCACCAAAAGCUG | 13 |
| sgRNA | UCCCuuagGGGAUUAGAACUUGAGUGAAGGUGGGCUGCUU | |
| | GCAUCAGCCUAAUGUCGAGAAGUGCUUUCUUCGGAAAG | |
| | UAACCCUCGAAACAAAUUCAUUUUUCCUCUCCAAUUCUG | |
| | CACAAgaaaGUUGCAGAACCCGAAUAGacgaaUGAAGGAAUG | |
| | CAACNNNNNNNNNNNNNNNNNNNN | |
| MS1 | CUUCACUGAUAAAGUGGAGAACCGCUUCACCAAAAGCUG | 149 |
| | UCCCUUAGGGGAUUAGAACUUGAGUGAAGGUGGGCUGC | |
| | UUGCAUCAGCCUAAUGUCGAGAAGUGCUUUCUUCGGAA | |
| | AGUAACCCUCGAAACAAAUUCAGUGCUCCUCUCCAAUUC | |
| | UGCACAAgaaaGUUGCAGAACCCGAAUAGAGCAAUGAAGG | |
| | AAUGCAACNNNNNNNNNNNNNNNNNNNN | |
| MS1/MS2 | CUUCACUGAUAAAGUGGAGAACCGCUUCACCAAAAGCUG | 150 |
| | UCCCUUAGGGGAUUAGAACUUGAGUGAAGGUGGGCUGC | |
| | UUGCAUCAGCCUAAUGUCGAGAAGUGCUUUCUUCGGAA | |
| | AGUAACCCUCGAAACAAAUUCAGUGCUCCUCUCCAAUUC | |
| | UGCACAAgaaaGUUGCAGAACCCGAAUAGAGCAAUGAAGG | |
| | AAUGCAACNNNNNNNNNNNNNNNNNNNNUUUUAUUUUU | |
| | U | |
| MS1/MS2/MS3 | ACCGCUUCACCAAAAGCUGUCCCUUAGGGGAUUAGAACU | 151 |
| (ge3.0) | UGAGUGAAGGUGGGCUGCUUGCAUCAGCCUAAUGUCGA | |
| | GAAGUGCUUUCUUCGGAAAGUAACCCUCGAAACAAAUUC | |
| | AGUGCUCCUCUCCAAUUCUGCACAAgaaaGUUGCAGAACC | |
| | CGAAUAGAGCAAUGAAGGAAUGCAACNNNNNNNNNNNN | |
| | NNNNNNNNUUUUAUUUUUU | |
| MS2/MS3/MS4 | ACCGCUUCACCAAAAGCUGUCCCUUAGGGGAUUAGAACU | 152 |
| (ge4.0) | UGAGUGAAGGUGGGCUGCUUGCAUCAGCCUAAUGUCGA | |
| | GAAGUGCUUUCUUCGGAAAGUAACCCUCGAAACAAAgaaa | |
| | GGAAUGCAACNNNNNNNNNNNNNNNNNNNNUUUUAUUU | |
| | UUU | |
| MS2/MS3/MS4/ | ACCGCUUCACUUAGAGUGAAGGUGGGCUGCUUGCAUCAG | 153 |
| MS5 | CCUAAUGUCGAGAAGUGCUUUCUUCGGAAAGUAACCCUC | |
| (ge4.1) | GAAACAAAgaaaGGAAUGCAACNNNNNNNNNNNNNNNNNN | |
| | NNUUUUAUUUUUU | |
| MS1/MS3-1 | GAUAAAGUGGAGAACCGCUUCACCAAAAGCUGUCCCUUA | 154 |
| | GGGGAUUAGAACUUGAGUGAAGGUGGGCUGCUUGCAUC | |
| | AGCCUAAUGUCGAGAAGUGCUUUCUUCGGAAAGUAACCC | |
| | UCGAAACAAAUUCAGUGCUCCUCUCCAAUUCUGCACAAg | |
| | aaaGUUGCAGAACCCGAAUAGAGCAAUGAAGGAAUGCAAC | |
| | NNNNNNNNNNNNNNNNNNNN | |
| MS1/MS3-2 | UGGAGAACCGCUUCACCAAAAGCUGUCCCUUAGGGGAUU | 155 |
| | AGAACUUGAGUGAAGGUGGGCUGCUUGCAUCAGCCUAA | |
| | UGUCGAGAAGUGCUUUCUUCGGAAAGUAACCCUCGAAAC | |
| | AAAUUCAGUGCUCCUCUCCAAUUCUGCACAAgaaaGUUGC | |
| | AGAACCCGAAUAGAGCAAUGAAGGAAUGCAACNNNNNN | |
| | NNNNNNNNNNNNNN | |
| MS1/MS3-3 | ACCGCUUCACCAAAAGCUGUCCCUUAGGGGAUUAGAACU | 156 |
| | UGAGUGAAGGUGGGCUGCUUGCAUCAGCCUAAUGUCGA | |
| | GAAGUGCUUUCUUCGGAAAGUAACCCUCGAAACAAAUUC | |
| | AGUGCUCCUCUCCAAUUCUGCACAAgaaaGUUGCAGAACC | |
| | CGAAUAGAGCAAUGAAGGAAUGCAACNNNNNNNNNNNN | |
| | NNNNNNNN | |
| MS1/MS4*-1 | CUUCACUGAUAAAGUGGAGAACCGCUUCACCAAAAGCUG | 157 |
| | UCCCUUAGGGGAUUAGAACUUGAGUGAAGGUGGGCUGC | |
| | UUGCAUCAGCCUAAUGUCGAGAAGUGCUUUCUUCGGAA | |
| | AGUAACCCUCGAAACAAAUUCAGUGCUCCUCUCCAAUUC | |
| | gaaaGAACCCGAAUAGAGCAAUGAAGGAAUGCAACNNNNN | |
| | NNNNNNNNNNNNNNN | |
| MS1/MS4*-2 | CUUCACUGAUAAAGUGGAGAACCGCUUCACCAAAAGCUG | 158 |
| | UCCCUUAGGGGAUUAGAACUUGAGUGAAGGUGGGCUGC | |
| | UUGCAUCAGCCUAAUGUCGAGAAGUGCUUUCUUCGGAA | |
| | AGUAACCCUCGAAACAAAUUCAGUGCUCCUCUCgaaaGAA | |
| | UAGAGCAAUGAAGGAAUGCAACNNNNNNNNNNNNNNNN | |
| | NNNN | |
| MS1/MS4*-3 | CUUCACUGAUAAAGUGGAGAACCGCUUCACCAAAAGCUG | 159 |
| | UCCCUUAGGGGAUUAGAACUUGAGUGAAGGUGGGCUGC | |
| | UUGCAUCAGCCUAAUGUCGAGAAGUGCUUUCUUCGGAA | |
| | AGUAACCCUCGAAACAAAUUCAGUGCUgaaaAGCAAUGAA | |
| | GGAAUGCAACNNNNNNNNNNNNNNNNNNNN | |
| MS1/MS5-1 | CUUCACUGAUAAAGUGGAGAACCGCUUCACCAAAAGCUG | 160 |
| | UuuagAUUAGAACUUGAGUGAAGGUGGGCUGCUUGCAUCA | |
| | GCCUAAUGUCGAGAAGUGCUUUCUUCGGAAAGUAACCCU | |
| | CGAAACAAAUUCAGUGCUCCUCUCCAAUUCUGCACAAgaa | |
| | aGUUGCAGAACCCGAAUAGAGCAAUGAAGGAAUGCAACN | |
| | NNNNNNNNNNNNNNNNNNN | |
| MS1/MS5-2 | CUUCACUGAUAAAGUGGAGAACCGCUUCACCAAAAGCuua | 161 |
| | gGAACUUGAGUGAAGGUGGGCUGCUUGCAUCAGCCUAAU | |
| | GUCGAGAAGUGCUUUCUUCGGAAAGUAACCCUCGAAACA | |
| | AAUUCAGUGCUCCUCUCCAAUUCUGCACAAgaaaGUUGCA | |
| | GAACCCGAAUAGAGCAAUGAAGGAAUGCAACNNNNNNN | |
| | NNNNNNNNNNNNN | |
| MS1/MS5-3*- | CUUCACUGAUAAAGUGGAGAACCGCUUCACCAAuuagUUG | 162 |
| 2 | AGUGAAGGUGGGCUGCUUGCAUCAGCCUAAUGUCGAGA | |
| | AGUGCUUUCUUCGGAAAGUAACCCUCGAAACAAAUUCAG | |
| | UGCUCCUCUCCAAUUCUGCACAAgaaaGUUGCAGAACCCG | |
| | AAUAGAGCAAUGAAGGAAUGCAACNNNNNNNNNNNNNN | |
| | NNNNNN | |
| MS1/MS2/MS4 | CUUCACUGAUAAAGUGGAGAACCGCUUCACCAAAAGCUG | 163 |
| | UCCCUUAGGGGAUUAGAACUUGAGUGAAGGUGGGCUGC | |
| | UUGCAUCAGCCUAAUGUCGAGAAGUGCUUUCUUCGGAA | |
| | AGUAACCCUCGAAACAAAUUCAGUGCUCCUCUCgaaaGAA | |
| | UAGAGCAAUGAAGGAAUGCAACNNNNNNNNNNNNNNNN | |
| | NNNNUUUUAUUUU | |
| MS1/MS3- | ACCGCUUCACCAAAAGCUGUCCCUUAGGGGAUUAGAACU | 164 |
| 3/MS4*-2 | UGAGUGAAGGUGGGCUGCUUGCAUCAGCCUAAUGUCGA | |
| | GAAGUGCUUUCUUCGGAAAGUAACCCUCGAAACAAAUUC | |
| | AGUGCUCCUCUCgaaaGAAUAGAGCAAUGAAGGAAUGCAA | |
| | CNNNNNNNNNNNNNNNNNNNN | |
| MS1/MS2/MS5- | CUUCACUGAUAAAGUGGAGAACCGCUUCACCAAuuagUUG | 165 |
| 3 | AGUGAAGGUGGGCUGCUUGCAUCAGCCUAAUGUCGAGA | |
| | AGUGCUUUCUUCGGAAAGUAACCCUCGAAACAAAUUCAG | |
| | UGCUCCUCUCCAAUUCUGCACAAgaaaGUUGCAGAACCCG | |
| | AAUAGAGCAAUGAAGGAAUGCAACNNNNNNNNNNNNNN | |
| | NNNNNNUUUUAUUUU | |
| MS1/MS3- | ACCGCUUCACCAAuuagUUGAGUGAAGGUGGGCUGCUUGC | 166 |
| 3/MS5-3 | AUCAGCCUAAUGUCGAGAAGUGCUUUCUUCGGAAAGUA | |
| | ACCCUCGAAACAAAUUCAGUGCUCCUCUCCAAUUCUGCA | |
| | CAAgaaaGUUGCAGAACCCGAAUAGAGCAAUGAAGGAAUG | |
| | CAACNNNNNNNNNNNNNNNNNNNN | |
| MS1/MS4*- | CUUCACUGAUAAAGUGGAGAACCGCUUCACCAAuuagUUG | 167 |
| 2/MS5-3 | AGUGAAGGUGGGCUGCUUGCAUCAGCCUAAUGUCGAGA | |
| | AGUGCUUUCUUCGGAAAGUAACCCUCGAAACAAAUUCAG | |
| | UGCUCCUCUCgaaaGAAUAGAGCAAUGAAGGAAUGCAACN | |
| | NNNNNNNNNNNNNNNNNNN | |
| MS1/MS2/MS3- | ACCGCUUCACCAAAAGCUGUCCCuuagGGGAUUAGAACUU | 168 |
| 3/MS4*-2 | GAGUGAAGGUGGGCUGCUUGCAUCAGCCUAAUGUCGAG | |
| | AAGUGCUUUCUUCGGAAAGUAACCCUCGAAACAAAUUCA | |
| | GUGCUCCUCUCgaaaGAAUAGAGCAAUGAAGGAAUGCAAC | |
| | NNNNNNNNNNNNNNNNNNNNUUUUAUUUU | |
| MS1/MS2/MS3- | ACCGCUUCACCAAuuagUUGAGUGAAGGUGGGCUGCUUGC | 169 |
| 3/MS5-3 | AUCAGCCUAAUGUCGAGAAGUGCUUUCUUCGGAAAGUA | |
| | ACCCUCGAAACAAAUUCAGUGCUCCUCUCCAAUUCUGCA | |
| | CAAgaaaGUUGCAGAACCCGAAUAGAGCAAUGAAGGAAUG | |
| | CAACNNNNNNNNNNNNNNNNNNNNUUUUAUUUU | |
| MS1/MS2/MS4*- | CUUCACUGAUAAAGUGGAGAACCGCUUCACCAAuuagUUG | 170 |
| 2/MS5-3 | AGUGAAGGUGGGCUGCUUGCAUCAGCCUAAUGUCGAGA | |
| | AGUGCUUUCUUCGGAAAGUAACCCUCGAAACAAAUUCAG | |
| | UGCUCCUCUCgaaaGAAUAGAGCAAUGAAGGAAUGCAACN | |
| | NNNNNNNNNNNNNNNNNNNUUUUAUUUU | |
| MS1/MS3- | ACCGCUUCACCAAuuagUUGAGUGAAGGUGGGCUGCUUGC | 171 |
| 3/MS4*- | AUCAGCCUAAUGUCGAGAAGUGCUUUCUUCGGAAAGUA | |
| 2/MS5-3 | ACCCUCGAAACAAAUUCAGUGCUCCUCUCgaaaGAAUAGA | |
| | GCAAUGAAGGAAUGCAACNNNNNNNNNNNNNNNNNNNN | |
| MS1/MS2/MS3- | ACCGCUUCACCAAuuagUUGAGUGAAGGUGGGCUGCUUGC | 172 |
| 3/MS4*- | AUCAGCCUAAUGUCGAGAAGUGCUUUCUUCGGAAAGUA | |
| 2/MS5-3 | ACCCUCGAAACAAAUUCAGUGCUCCUCUCgaaaGAAUAGA | |
| | GCAAUGAAGGAAUGCAACNNNNNNNNNNNNNNNNNNNN | |
| | UUUUAUUUU | |

In addition, a mature form gRNA was produced by removing the modification site MS1 from the canonical gRNA, and the specific sequences thereof are shown in Table 5.

TABLE 5

| | | SEQ ID |
|---|---|---|
| gRNA | Sequence (5′ to 3′) | NO |
| Mature form | CUUCACUGAUAAAGUGGAGAACCGCUUCACCAAAAGCUG | 173 |
| gRNA | UCCCuuagGGGAUUAGAACUUGAGUGAAGGUGGGCUGCUU | |
| | GCAUCAGCCUAAUGUCGAGAAGUGCUUUCUUCGGAAAG | |
| | UAACCCUCGAAACAAAUUCAUUUgaaaGAAUGAAGGAAUG | |
| | CAACNNNNNNNNNNNNNNNNNNNN | |
| MS3-1 | GAUAAAGUGGAGAACCGCUUCACCAAAAGCUGUCCCuuag | 174 |
| | GGGAUUAGAACUUGAGUGAAGGUGGGCUGCUUGCAUCA | |
| | GCCUAAUGUCGAGAAGUGCUUUCUUCGGAAAGUAACCCU | |
| | CGAAACAAAUUCAUUUgaaaGAAUGAAGGAAUGCAACNNN | |
| | NNNNNNNNNNNNNNNNN | |
| MS3-2 | UGGAGAACCGCUUCACCAAAAGCUGUCCCuuagGGGAUUA | 175 |
| | GAACUUGAGUGAAGGUGGGCUGCUUGCAUCAGCCUAAU | |
| | GUCGAGAAGUGCUUUCUUCGGAAAGUAACCCUCGAAACA | |
| | AAUUCAUUUgaaaGAAUGAAGGAAUGCAACNNNNNNNNNN | |
| | NNNNNNNNNN | |
| MS3-3 | ACCGCUUCACCAAAAGCUGUCCCuuagGGGAUUAGAACUU | 176 |
| | GAGUGAAGGUGGGCUGCUUGCAUCAGCCUAAUGUCGAG | |
| | AAGUGCUUUCUUCGGAAAGUAACCCUCGAAACAAAUUCA | |
| | UUUgaaaGAAUGAAGGAAUGCAACNNNNNNNNNNNNNNN | |
| | NNNNN | |
| MS4-1 | CUUCACUGAUAAAGUGGAGAACCGCUUCACCAAAAGCUG | 177 |
| | UCCCuuagGGGAUUAGAACUUGAGUGAAGGUGGGCUGCUU | |
| | GCAUCAGCCUAAUGUCGAGAAGUGCUUUCUUCGGAAAG | |
| | UAACCCUCGAAACAAAUUCAUgaaaAUGAAGGAAUGCAAC | |
| | NNNNNNNNNNNNNNNNNNNN | |
| MS4-2 | CUUCACUGAUAAAGUGGAGAACCGCUUCACCAAAAGCUG | 178 |
| | UCCCuuagGGGAUUAGAACUUGAGUGAAGGUGGGCUGCUU | |
| | GCAUCAGCCUAAUGUCGAGAAGUGCUUUCUUCGGAAAG | |
| | UAACCCUCGAAACAAAUUCgaaaGAAGGAAUGCAACNNNN | |
| | NNNNNNNNNNNNNNNN | |
| MS4-3 | CUUCACUGAUAAAGUGGAGAACCGCUUCACCAAAAGCUG | 179 |
| | UCCCuuagGGGAUUAGAACUUGAGUGAAGGUGGGCUGCUU | |
| | GCAUCAGCCUAAUGUCGAGAAGUGCUUUCUUCGGAAAG | |
| | UAACCCUCGAAACAAAgaaaGGAAUGCAACNNNNNNNNNN | |
| | NNNNNNNNNN | |
| MS5-1 | CUUCACUGAUAAAGUGGAGAACCGCUUCACCAAAAGCUG | 180 |
| | UuuagAUUAGAACUUGAGUGAAGGUGGGCUGCUUGCAUCA | |
| | GCCUAAUGUCGAGAAGUGCUUUCUUCGGAAAGUAACCCU | |
| | CGAAACAAAUUCAUUUgaaaGAAUGAAGGAAUGCAACNNN | |
| | NNNNNNNNNNNNNNNNN | |
| MS5-2 | CUUCACUGAUAAAGUGGAGAACCGCUUCACCAAAAGCUu | 181 |
| | uagAGAACUUGAGUGAAGGUGGGCUGCUUGCAUCAGCCU | |
| | AAUGUCGAGAAGUGCUUUCUUCGGAAAGUAACCCUCGA | |
| | AACAAAUUCAUUUgaaaGAAUGAAGGAAUGCAACNNNNNN | |
| | NNNNNNNNNNNNNN | |
| MS5-3 | CUUCACUGAUAAAGUGGAGAACCGCUUCACCAAuuagUUG | 182 |
| | AGUGAAGGUGGGCUGCUUGCAUCAGCCUAAUGUCGAGA | |
| | AGUGCUUUCUUCGGAAAGUAACCCUCGAAACAAAUUCAU | |
| | UUgaaaGAAUGAAGGAAUGCAACNNNNNNNNNNNNNNNN | |
| | NNNN | |
| MS3-3/MS4-3 | ACCGCUUCACCAAAAGCUGUCCCuuagGGGAUUAGAACUU | 183 |
| | GAGUGAAGGUGGGCUGCUUGCAUCAGCCUAAUGUCGAG | |
| | AAGUGCUUUCUUCGGAAAGUAACCCUCGAAACAAAgaaaG | |
| | GAAUGCAACNNNNNNNNNNNNNNNNNNNN | |
| MS3-3/MS5-3 | ACCGCUUCACCAAuuagUUGAGUGAAGGUGGGCUGCUUGC | 184 |
| | AUCAGCCUAAUGUCGAGAAGUGCUUUCUUCGGAAAGUA | |
| | ACCCUCGAAACAAAUUCAUUUgaaaGAAUGAAGGAAUGCA | |
| | ACNNNNNNNNNNNNNNNNNNNN | |
| MS4-3/MS5-3 | CUUCACUGAUAAAGUGGAGAACCGCUUCACCAAuuagUUG | 185 |
| | AGUGAAGGUGGGCUGCUUGCAUCAGCCUAAUGUCGAGA | |
| | AGUGCUUUCUUCGGAAAGUAACCCUCGAAACAAAgaaaGG | |
| | AAUGCAACNNNNNNNNNNNNNNNNNNNN | |
| MS3-3/MS4- | ACCGCUUCACCAAuuagUUGAGUGAAGGUGGGCUGCUUGC | 186 |
| 3/MS5-3 | AUCAGCCUAAUGUCGAGAAGUGCUUUCUUCGGAAAGUA | |
| | ACCCUCGAAACAAAgaaaGGAAUGCAACNNNNNNNNNNNN | |
| | NNNNNNNN | |

The sequence indicated by NNNNNNNNNNNNNNNNNNNN in Tables 4 and 5 refers to any guide sequence (spacer sequence) that can hybridize to the target sequence. The guide sequence may be appropriately designed by those skilled in the art depending on a desired target gene and/or a target sequence, and is not limited to a specific sequence of a particular length.

### Example 1.2. Cas12f1 and TARGET Systems

UnCas12f1 and CWCas21f1 were used together with the guide RNA of Example 1.1. PCR amplification was performed using the human codon-optimized nucleic acid sequence (SEQ ID NOs: 10 and 6) of the protein as a template, and cloning was performed according to a desired cloning sequence into a vector having a promoter capable of expression in a eukaryotic system and a poly(A) signal sequence using the Gibson assembly method. After cloning, the sequence of the obtained recombinant plasmid vector was finally identified by the Sanger sequencing method. The nucleic acid construct thus produced was cloned into the pMAL-c2 plasmid vector, and transformed into BL21(DE3) E. coli cells. The transformed E. coli colonies were grown in LB broth at 37° C. until the optical density reached 0.7. The transformed E. coli cells were cultured at 18° C. overnight in the presence of 0.1 mM isopropylthio-β-D-galactoside. Thereafter, the cultured cells were collected by centrifugation at 3,500 g for 30 minutes, and the collected cells were resuspended in 20 mM Tris-HCl (pH 7.6), 500 mM NaCl, 5 mM β-mercaptoethanol, and 5% glycerol. The cells were lysed in a lysis buffer and then disrupted by sonication. The sample containing the disrupted cells was centrifuged at 15,000 g for 30 minutes, and the supernatant obtained was filtered through a 0.45 μm syringe filter (Millipore). The filtered supernatant was loaded onto a Ni²⁺-affinity column using an FPLC purification system (KTA Purifier, GE Healthcare). The bound fractions were eluted with a gradient of 80 to 400 mM imidazole, 20 mM Tris-HCl (pH 7.5).

The eluted proteins were cleaved by treatment with TEV protease for 16 hours. The cleaved proteins were purified on a heparin column with a linear gradient of 0.15-1.6 M NaCl. The recombinant Cas12f1 variant protein purified on the heparin column was dialyzed against a solution of 20 mM Tris (pH 7.6), 150 mM NaCl, 5 mM β-mercaptoethanol, and 5% glycerol. The dialyzed protein was purified by passing it through an MBP column, and then repurified on a monoS column (GE Healthcare) or EnrichS with a linear gradient of 0.5-1.2 M NaCl.

The repurified proteins were collected and dialyzed against a solution of 20 mM Tris (pH 7.6), 150 mM NaCl, 5 mM β-mercaptoethanol, and 5% glycerol to purify the hypercompact gene editing proteins (miniature endonucleases) used in the present disclosure. The concentration of the produced hypercompact gene editing proteins was quantified by the Bradford quantitative method using bovine serum albumin (BSA) as a standard and measured electrophoretically on a coomassie blue-stained SDS-PAGE gel.

### Comparative Example 1. SaCas9 System

Each guide sequence was cloned into a plasmid containing U6 promoter and SaCas9 scaffold. Then, each sequence from the U6 promoter to the guide RNA was inserted into the plasmid encoding SaCas9 to produce a one-vector module. Information on the protospacer sequence and PAM sequence of F68 and R84 in the SaCas9 system are as follows. For F68, the protospacer sequence is 5′-GTGTATTGCTTGTACTACTCA-3′ (SEQ ID NO: 401), and the PAM sequence is 5′-CTGAAT-3′. In addition, for R84, the protospacer sequence is 5′-GTGTTATTACTTGCTACTGCA-3′ (SEQ ID NO: 402), and the PAM sequence is 5′-GAGAGT-3′.

### Example 1.3. Selection of Target Sequence

The regions 2000 bp upstream and 2000 bp downstream of exon 51 were set as target regions for deletion of exon 51, and the target regions were referred to as the front region (F region) and the rear region (R region), respectively. Protospacer sequences were selected from the above regions, and the selected protospacer sequences are summarized in Table 6. The protospacer regions in the F region were numbered with F, and the protospacer regions in the R region were numbered with R. The PAM sequence adjacent to each protospacer sequence is also shown therein.

TABLE 6

| | | PAM | Protospacer sequence | |
|---|---|---|---|---|
| No. | Name | (TTTR) | (5′ to 3′) | SEQ ID NO |
| 1 | F102 | TTTA | AGGTTGTCTCCTCATTAGAG | 190 |
| 2 | F104 | TTTA | ATATTCTTAGAATCGTTCAC | 191 |
| 3 | F108 | TTTG | CCACCAATCTTCTGGTTATA | 192 |
| 4 | F109 | TTTA | TAGACAACAGACTCAAGAGC | 193 |
| 5 | F110 | TTTG | ACTGACTACTCCCCAAGTAT | 194 |
| 6 | F111 | TTTG | GGGCCTTATCTCCAGTTTCT | 195 |
| 7 | F114 | TTTA | TAAACGCCTGGCTAGTAAGA | 196 |
| 8 | F115 | TTTA | TAAAAACAGTAATACCTAAC | 197 |
| 9 | F118 | TTTG | CCTTGCTTACTGCTTATTGC | 198 |
| 10 | F119 | TTTG | TTCAGTACTAGCAATAAGCA | 199 |
| 11 | F121 | TTTA | TTTAATGACTTTGAAACAGT | 200 |
| 12 | F123 | TTTG | AAACAGTATTTCATGTCTAA | 201 |
| 13 | F124 | TTTA | GACATGAAATACTGTTTCAA | 202 |
| 14 | F128 | TTTA | AGAAAATATTGTATCTTGGT | 203 |
| 15 | F130 | TTTA | TTTCAGATTGTTAGTAAACT | 204 |
| 16 | F131 | TTTA | TACTGGGAGCAGTTTATCAT | 205 |
| 17 | F133 | TTTG | ATTTCCCTAGGGTCCAGCTT | 206 |
| 18 | F134 | TTTG | AAGCTGGACCCTAGGGAAAT | 207 |
| 19 | F135 | TTTA | AAATTCCCTTGAATAGGAAG | 208 |
| 20 | F136 | TTTA | AATCAGAAAGAAGATCTTAT | 209 |
| 21 | F138 | TTTA | TTCAAGAAAAAACAAAGGCA | 210 |
| 22 | F140 | TTTA | CCACTTCCACAATGTATATG | 211 |
| 23 | F141 | TTTA | ACTTAAGTTACTTGTCCAGG | 212 |
| 24 | F142 | TTTG | CTCATTCTCATGCCTGGACA | 213 |
| 25 | F143 | TTTA | AAAAATTGTTAAATGTATAT | 214 |
| 26 | F147 | TTTA | ATTGAAGAGTAACAATTTGA | 215 |
| 27 | F148 | TTTG | ACTTATTGTTATTGAAATTG | 216 |
| 28 | F43 | TTTA | GTATCAATTCACACCAGCAA | 217 |
| 29 | R52 | TTTG | CTCTCCTAGACCATTTCCCA | 218 |
| 30 | R55 | TTTA | CTGAGAGAGAAACAGTTGCC | 219 |
| 31 | R56 | TTTG | TATCCTTGATTATACTTAGG | 220 |
| 32 | R57 | TTTG | CATTAATTTATATCCTTGAT | 221 |
| 33 | R58 | TTTA | TTATTTGCATTAATTTATAT | 222 |
| 34 | R59 | TTTA | ACTGAGACAACTATTCTTGT | 223 |
| 35 | R60 | TTTA | GTAAATTTAACTGAGACAAC | 224 |
| 36 | R61 | TTTA | AAATACCAGGTTGTTTAGTA | 225 |
| 37 | R62 | TTTA | TACCAAATAAGTCACTCAAC | 226 |
| 38 | R63 | TTTG | AATGTAAATAGCTCAGTTGA | 227 |
| 39 | R64 | TTTG | GCACTACGCAGCCCAACATA | 228 |
| 40 | R65 | TTTG | ATTCTGCAATATGTTGGGCT | 229 |
| 41 | R66 | TTTA | CCTTCTCCAATGATAAGGAT | 230 |
| 42 | R68 | TTTA | AGTTATAGCTCTCTTTCAAT | 231 |
| 43 | R69 | TTTA | AAATTACCCTAGATCTTAAA | 232 |
| 44 | R70 | TTTG | TGCAATGCCATGTTCAAATG | 233 |
| 45 | R71 | TTTA | AACATGGCATTGCATAAATG | 234 |
| 46 | R73 | TTTG | CCAAATGGATTACACTGAGG | 235 |
| 47 | R74 | TTTA | GTAAAATTATGACATCAACT | 236 |
| 48 | R75 | TTTG | CCTAGCTAGATCAAATATAC | 237 |
| 49 | R76 | TTTA | ATCTAGCTAGGTAAACCATA | 238 |
| 50 | R77 | TTTA | TATTTGTGAATGATTAAGAA | 239 |
| 51 | R79 | TTTA | ATGTATAACAATTCCAACAT | 240 |
| 52 | R82 | TTTG | GAAAGGCTTGAAAGCTGTTA | 241 |
| 53 | R84 | TTTA | TATTTCTTTAGAAAGGCTTG | 242 |
| 54 | R85 | TTTG | ATACCTAAATACCTTCAGCA | 243 |
| 55 | R86 | TTTG | AAAAAACAAGAAGTGAGGCA | 244 |
| 56 | R89 | TTTA | TAAAATAAACTTCACCAATT | 245 |
| 57 | R90 | TTTA | CAAAGAAATTCCCCTTACTC | 246 |
| 58 | R91 | TTTA | TTCCTATGGAATTGGTGAAG | 247 |
| 59 | R92 | TTTA | AAGGCCTTGAGCTTGAATAC | 248 |
| 60 | R93 | TTTG | AATGCAGGTTGTTTACTATC | 249 |
| 61 | R94 | TTTG | CAAATGATGATTGTGGTCAA | 250 |
| 62 | R95 | TTTA | CAAATGGCACTGAATTCAGG | 251 |
| 63 | R97 | TTTA | TATACATCCATATCCATAAC | 252 |
| 64 | R98 | TTTA | CATATATTTATATACATCCA | 253 |
| 65 | R99 | TTTA | CTTCTGCTTTAAAAAAAGTA | 254 |

In addition, the regions 2000 bp to 3000 bp upstream of exon 51 and the regions 2000 bp to 3000 bp downstream of exon 51 were set as additional target regions for deletion of exon 51. The specific sequences are shown in Table 7 below. Similarly, the protospacer regions in the F region were numbered with F, and the protospacer regions in the R region was numbered with R. The PAM sequence adjacent to each protospacer sequence is also shown therein.

TABLE 7

| | | PAM | Protospacer sequence | |
|---|---|---|---|---|
| No. | Name | (TTTR) | (5′ to 3′) | SEQ ID NO |
| 1 | F1 | TTTA | TCATCCCAATGGCATATTTA | 255 |
| 2 | F2 | TTTA | GTCATCATGAACCATTCTCT | 256 |
| 3 | F3 | TTTG | AGTCTCAGGCCCTGGCCTTG | 257 |
| 4 | F4 | TTTG | GAAAAAGACAGAAAGGAAGA | 258 |
| 5 | F5 | TTTA | TTGAAATTTAGTGTGACATG | 259 |
| 6 | F6 | TTTA | GTGTGACATGATCATTTCCA | 260 |
| 7 | F7 | TTTG | ACCCTCAAATAAGAGCAGAG | 261 |
| 8 | F8 | TTTG | AGGGTCAAATTTTCAATAAG | 262 |
| 9 | F9 | TTTA | AAAAGTGACTTATTGAAAAT | 263 |
| 10 | F10 | TTTA | AAAAAGCATTTTCTGACACT | 264 |
| 11 | F11 | TTTA | GGAGGATTTCAAGCTTATGG | 265 |
| 12 | F12 | TTTA | GGAACACTCCATCCACATTT | 266 |
| 13 | F13 | TTTG | CCAGTCAGCCTGGTGTTCTG | 267 |
| 14 | F14 | TTTA | ATTTAGCTCTCTTTTCATCC | 268 |
| 15 | F15 | TTTA | GCTCTCTTTTCATCCTCACA | 269 |
| 16 | F16 | TTTA | GCTCCTTTCCTGGAATCCCT | 270 |
| 17 | F17 | TTTG | CTTCCATGATTTCTTCTCCT | 271 |
| 18 | F18 | TTTG | TGTCACAGTCCAGGAGAAGA | 272 |
| 19 | F19 | TTTA | CATTACATTTAATTCTTTCT | 273 |
| 20 | F20 | TTTA | ATTCTTTCTAGAAAGAGCCT | 274 |
| 21 | F21 | TTTG | TTTCTCAAAGCTATCTGACT | 275 |
| 22 | F22 | TTTA | TTGCCTCACTGTTACTGCCT | 276 |
| 23 | F23 | TTTG | TGTCACGAAACAATGATTGA | 277 |
| 24 | F24 | TTTA | ATTGTCAGAGAGAATAAAAA | 278 |
| 25 | F25 | TTTA | TTCTCTCTGACAATTAAAAC | 279 |
| 26 | F26 | TTTG | ATCAATGCAGACAGAAAAAA | 280 |
| 27 | R1 | TTTG | ACATCTATGAGCCTCAGTTA | 281 |
| 28 | R2 | TTTG | AATCCTTGTTCTGCTACTTA | 282 |
| 29 | R3 | TTTG | ATTCCTAAGCTTGTGTTATT | 283 |
| 30 | R4 | TTTA | GTGATTTGTATGTAGATGTA | 284 |
| 31 | R5 | TTTG | TATGTAGATGTAGATGTAGT | 285 |
| 32 | R6 | TTTA | TGGTTGCTATGTACTGATAC | 286 |
| 33 | R7 | TTTA | CAATACCATATTGAGTTATA | 287 |
| 34 | R8 | TTTG | GTAAATAAAAGTCCTGGGAG | 288 |
| 35 | R9 | TTTA | TTTACCAAAGGAAACAATAT | 289 |
| 36 | R10 | TTTA | CCAAAGGAAACAATATTTTA | 290 |
| 37 | R11 | TTTA | AACATTATAAAATATTGTTT | 291 |
| 38 | R12 | TTTA | TAATGTTTAAAGCCCAGGTT | 292 |
| 39 | R13 | TTTA | AAGCCCAGGTTTTGAAGTTA | 293 |
| 40 | R14 | TTTG | AACCCAGACAATGTAACTTC | 294 |
| 41 | R15 | TTTG | AAGTTACATTGTCTGGGTTC | 295 |
| 42 | R16 | TTTA | TAGCTAGATAAACTTGGGCT | 296 |
| 43 | R17 | TTTA | TCTAGCTATAAAATGGGGAT | 297 |
| 44 | R18 | TTTG | TCATCAGGATTAAGTTGGTT | 298 |
| 45 | R19 | TTTG | TTCAATACTAGCAGTAAGCA | 299 |
| 46 | R20 | TTTG | CCTTGCTTACTGCTTACTGC | 300 |
| 47 | R21 | TTTG | GTTCCACCACGAACTCTAGA | 301 |
| 48 | R22 | TTTG | CATAATACAAATGCCATCAT | 302 |
| 49 | R23 | TTTG | TATTATGCAAACTGTATATC | 303 |
| 50 | R24 | TTTG | TGAGTCCAGCATTTAGGGAA | 304 |
| 51 | R25 | TTTA | GGGAAGCCATTGATGTGCTC | 305 |
| 52 | R26 | TTTA | GAGAACTCTGGAGACTACTG | 306 |
| 53 | R27 | TTTA | TTTAGAGAACTCTGGAGACT | 307 |
| 54 | R28 | TTTG | ATGCCCCCTCACAGAGATCG | 308 |
| 55 | R29 | TTTA | AAGACGATCTCTGTGAGGGG | 309 |
| 56 | R30 | TTTG | ACCTATATTTAAAGACGATC | 310 |
| 57 | R31 | TTTA | AATATAGGTCAAAAACTAAT | 311 |

Guide RNA was designed based on the selected protospacer sequences, and indel efficiency thereof was analyzed. Specifically, a cassette (U6 Promoter—Direct Repeat—gRNA; PCR amplicon) was constructed based on each of the selected protospacer sequences and the engineered gRNA (ge4.1 of SEQ ID NO: 153) in Table 4, and transfected into previously prepared HEK293 cells using FuGENE HD together with a Cas12f1 vector. Human codon-optimized nucleic acid encoding Un1 Cas12f1 was used for construction of the Cas12f1 vector. The transfected cells were incubated for 5 days, and gDNA (genomic DNA) was extracted therefrom. Then, PCR amplification was performed using the designed primers. The indel efficiency was measured therefrom, and is shown in Tables 8 and 9. The untreated group was used as a control group.

TABLE 8

| No. | Name | % Indel |
|---|---|---|
| 1 | F102 | 15.3 |
| 2 | F104 | 14.3 |
| 3 | F108 | 1.1 |
| 4 | F109 | 11 |
| 5 | F110 | 0 |
| 6 | F111 | 0.8 |
| 7 | F114 | 0 |
| 8 | F115 | 0 |
| 9 | F118 | 0 |
| 10 | F119 | 5.6 |
| 11 | F121 | 0.9 |
| 12 | F123 | 0.8 |
| 13 | F124 | 4.2 |
| 14 | F128 | 0.6 |
| 15 | F130 | 0.5 |
| 16 | F131 | 5.7 |
| 17 | F133 | 4.9 |
| 18 | F134 | 13 |
| 19 | F135 | 0 |
| 20 | F136 | 0 |
| 21 | F138 | 6.7 |
| 22 | F140 | 0 |
| 23 | F141 | 1.1 |
| 24 | F142 | 20.16 |
| 25 | F143 | 0 |
| 26 | F147 | 1.9 |
| 27 | F148 | 0 |
| 28 | F43 | 0 |
| 29 | R52 | 22.61 |
| 30 | R55 | 2.15 |
| 31 | R56 | 4.2 |
| 32 | R57 | 6.05 |
| 33 | R58 | 6.25 |
| 34 | R59 | 0.7 |
| 35 | R60 | 0.85 |
| 36 | R61 | 0.85 |
| 37 | R62 | 1 |
| 38 | R63 | 0.95 |
| 39 | R64 | 17.69 |
| 40 | R65 | 0.11 |
| 41 | R66 | 8.06 |
| 42 | R68 | 3.51 |
| 43 | R69 | 2.67 |
| 44 | R70 | 7.35 |
| 45 | R71 | 0.85 |
| 46 | R73 | 3.52 |
| 47 | R74 | 2.39 |
| 48 | R75 | 3.63 |
| 49 | R76 | 8.1 |
| 50 | R77 | 0.43 |
| 51 | R79 | 0.84 |
| 52 | R82 | 2.67 |
| 53 | R84 | 2.39 |
| 54 | R85 | 0.17 |
| 55 | R86 | 0.19 |
| 56 | R89 | 0.15 |
| 57 | R90 | 0.05 |
| 58 | R91 | 1.59 |
| 59 | R92 | 0.67 |
| 60 | R93 | 5.78 |
| 61 | R94 | 6.14 |
| 62 | R95 | 1.21 |
| 63 | R97 | 1.25 |
| 64 | R98 | 5.97 |
| 65 | R99 | 2.94 |

TABLE 9

| No. | Name | % Indel |
|---|---|---|
| 1 | F1 | 6.48 |
| 2 | F2 | 11.69 |
| 3 | F3 | 0.94 |
| 4 | F4 | 0 |
| 5 | F5 | 0.69 |
| 6 | F6 | 0 |
| 7 | F7 | 0.66 |
| 8 | F8 | 0.05 |
| 9 | F9 | 0.26 |
| 10 | F10 | 0 |
| 11 | F11 | 6.77 |
| 12 | F12 | 0 |
| 13 | F13 | 0.84 |
| 14 | F14 | 0 |
| 15 | F15 | 0.69 |
| 16 | F16 | 6.91 |
| 17 | F17 | 4.92 |
| 18 | F18 | 6.21 |
| 19 | F19 | 0 |
| 20 | F20 | 0.19 |
| 21 | F21 | 0.41 |
| 22 | F22 | 0.73 |
| 23 | F23 | 2.3 |
| 24 | F24 | 0.02 |
| 25 | F25 | 0.11 |
| 26 | F26 | 0.56 |
| 27 | R1 | 0.91 |
| 28 | R2 | 0.17 |
| 29 | R3 | 9.34 |
| 30 | R4 | 14.69 |
| 31 | R5 | 15.55 |
| 32 | R6 | 15.08 |
| 33 | R7 | 4.75 |
| 34 | R8 | 6.33 |
| 35 | R9 | 10.24 |
| 36 | R10 | 8.62 |
| 37 | R11 | 0 |
| 38 | R12 | 0 |
| 39 | R13 | 0 |
| 40 | R14 | 2.33 |
| 41 | R15 | 3.57 |
| 42 | R16 | 21.51 |
| 43 | R17 | 19.72 |
| 44 | R18 | 1.12 |
| 45 | R19 | 0.1 |
| 46 | R20 | 3.55 |
| 47 | R21 | 12.44 |
| 48 | R22 | 6.68 |
| 49 | R23 | 27.42 |
| 50 | R24 | 0.21 |
| 51 | R25 | 0.36 |
| 52 | R26 | 9.86 |
| 53 | R27 | 11.74 |
| 54 | R28 | 9.08 |
| 55 | R29 | 0.06 |
| 56 | R30 | 0 |
| 57 | R31 | 4.34 |

Subsequent experiments were performed by selecting target sequences that were confirmed to have high indel efficiency.

### Example 1.4. Analysis of Indel Efficiency Using CRISPR/Cas12f1 System

For the selected target sequences, the indel efficiency was analyzed using the CRISPR/Cas12f1 system comprising the engineered gRNA. Each of the cassettes (2 ug) constructed using ge4.0 and ge4.1 in Tables 4 and 5 was transfected into AC16 cells and HEK293 cells, and the indel efficiency was analyzed by repeating the experiment independently three times. The average indel efficiency (%) thereof is shown in Table 10.

TABLE 10

| | | HEK293 | AC16 |
|---|---|---|---|
| Name | gRNA version | Average indel (%) | Average indel (%) |
| F142 | ge4.0 | 53.98 | 48.08 |
| | ge4.1 | 49.98 | 19.18 |
| | wt | 0 | 0 |
| R52 | ge4.0 | 46.06 | 36.47 |
| | ge4.1 | 34.83 | 11.73 |
| | wt | 0 | 0 |
| F102 | ge4.1 | 40.13 | 14.46 |
| | wt | 0 | 0 |
| F104 | ge4.1 | 27.99 | 2.62 |
| | wt | 0 | 0 |
| F109 | ge4.1 | 24.87 | 7.09 |
| | wt | 0 | 0 |
| F131 | ge4.1 | 24.26 | 3.98 |
| | wt | 0 | 0 |
| F134 | ge4.1 | 19.54 | 2.11 |
| | wt | 0 | 0 |
| R57 | ge4.1 | 2.21 | 0.53 |
| | wt | 0 | 0 |
| R64 | ge4.1 | 36.41 | 5.33 |
| | wt | 0 | 0 |
| R66 | ge4.1 | 4.38 | 0.36 |
| | wt | 0 | 0 |
| R76 | ge4.1 | 41.89 | 14.33 |
| | wt | 0 | 0 |
| F2 | ge4.0 | 35.17 | 9.34 |
| | ge4.1 | 36.88 | 10.18 |
| | wt | 0 | 0 |
| R4 | ge4.0 | 0.00 | 0.00 |
| | ge4.1 | 43.65 | 12.25 |
| | wt | 0 | 0 |
| R5 | ge4.0 | 37.67 | 8.73 |
| | ge4.1 | 55.68 | 22.08 |
| | wt | 0 | 0 |
| R6 | ge4.0 | 21.60 | 5.96 |
| | ge4.1 | 57.97 | 13.18 |
| | wt | 0 | 0 |
| R16 | ge4.0 | 44.34 | 18.85 |
| | ge4.1 | 58.83 | 29.76 |
| | wt | 0 | 0 |
| R17 | ge4.0 | 4.04 | 0.35 |
| | ge4.1 | 31.66 | 7.40 |
| | wt | 0 | 0 |
| R23 | ge4.0 | 4.43 | 0.46 |
| | ge4.1 | 232.98 | 6.85 |
| | wt | 0 | 0 |

### Example 1.5. Optimization of Spacer and Analysis of Indel Efficiency

Optimization of spacer was performed to increase indel efficiency. Based on the PAM adjacent to the protospacer sequences of F142 and R52, vectors were constructed to have 19- to 25-mer guide sequences as shown in Table 11 and the relative indel efficiency depending on length of the guide sequence was analyzed. Here, relative means a relative value under the same conditions, as indel efficiency varies depending on the transfection time, vector type, and concentration. In order to stabilize the guide RNA and increase indel efficiency, U-rich tail (UR) and U6 were positioned at the 3′-end of each guide RNA, and the indel efficiency was analyzed. The results are shown in Table 11.

TABLE 11
F142

| | | SED ID | Indel |
|---|---|---|---|
| Name | Spacer sequence | NO | (%) |
| Control | — | — | 0.04 |
| 19-bp_UR | CUCAUUCUCAUGCCUGGACUUUUAUUUU | 336 | 20.38 |
| 20-bp_UR | CUCAUUCUCAUGCCUGGACAUUUUAUUUU | 337 | 30.01 |
| 21-bp_UR | CUCAUUCUCAUGCCUGGACAAUUUUAUUUU | 338 | 23.94 |
| 22-23- | CUCAUUCUCAUGCCUGGACAAGUUUUAUUUU | 339 | 15.56 |
| bp_UR | | | |
| 24_UR | CUCAUUCUCAUGCCUGGACAAGUAUUUUAUUUU | 340 | 13.61 |
| 25-bp_UR | CUCAUUCUCAUGCCUGGACAAGUAAUUUUAUUUU | 341 | 9.59 |
| 19-bp_U6 | CUCAUUCUCAUGCCUGGACUUUU | 342 | 13.41 |
| 20-bp_U6 | CUCAUUCUCAUGCCUGGACAUUUU | 343 | 14.36 |
| 21-bp_U6 | CUCAUUCUCAUGCCUGGACAAUUUU | 344 | 19.84 |
| 22-23- | CUCAUUCUCAUGCCUGGACAAGUUUU | 345 | 16.42 |
| bp_U6 | | | |
| 24-bp_U6 | CUCAUUCUCAUGCCUGGACAAGUAUUUU | 346 | 12.00 |
| 25-bp_U6 | CUCAUUCUCAUGCCUGGACAAGUAAUUUU | 347 | 9.87 |

R52

| | | SED ID | Indel |
|---|---|---|---|
| Name | Spacer sequence | NO | (%) |
| Control | — | — | 0.21 |
| 19-bp_UR | CUCUCCUAGACCAUUUCCCUUUUAUUUU | 348 | 16.18 |
| 20-bp_UR | CUCUCCUAGACCAUUUCCCAUUUUAUUUU | 349 | 15.72 |
| 21-bp_UR | CUCUCCUAGACCAUUUCCCACUUUUAUUUU | 350 | 17.98 |
| 22-bp_UR | CUCUCCUAGACCAUUUCCCACCUUUUAUUUU | 351 | 13.49 |
| 23 UR | CUCUCCUAGACCAUUUCCCACCAUUUUAUUUU | 352 | 3.54 |
| 24-25- | CUCUCCUAGACCAUUUCCCACCAGUUUUAUUUU | 353 | 9.92 |
| bp_UR | | | |
| 19-bp_U6 | CUCUCCUAGACCAUUUCCCUUUU | 354 | 18.90 |
| 20-bp_U6 | CUCUCCUAGACCAUUUCCCAUUUU | 355 | 20.51 |
| 21-bp U6 | CUCUCCUAGACCAUUUCCCACUUUU | 356 | 23.38 |
| 22bp_U6 | CUCUCCUAGACCAUUUCCCACCUUUU | 357 | 13.44 |
| 23-bp_U6 | CUCUCCUAGACCAUUUCCCACCAUUUU | 358 | 6.03 |
| 24-25- | CUCUCCUAGACCAUUUCCCACCAGUUUU | 359 | 16.15 |
| bp_U6 | | | |

As a result, 20-bp_UR showed the highest indel efficiency for the F142 spacer sequence, and 21-bp U6 showed the highest indel efficiency for the R52 spacer sequence. Next, the indel efficiency was analyzed in HEK293 and AC16 cells by the same method as Example 1.4 using the optimized spacer (20-bp_UR of F142 and 21-bp U6 of R52) and engineered gRNA (ge4.0 and ge4.1) or canonical gRNA. The results are shown in Table 12.

TABLE 12

| | | HEK293 | AC16 |
|---|---|---|---|
| Name | gRNA version | Average indel (%) | Average indel (%) |
| F142 | ge4.0 | 40.6 | 21.1 |
| (3 days) | ge4.1 | 41.5 | 7.0 |
| | wt | 0 | 0 |
| F142 | ge4.0 | 45.6 | 22.6 |
| (5 days) | ge4.1 | 44.5 | 9.0 |
| | wt | 0 | 0 |
| R52 | ge4.0 | 38.9 | 23.0 |
| (3 days) | ge4.1 | 34.4 | 5.5 |
| | wt | 0 | 0 |
| R52 | ge4.0 | 43.3 | 26.3 |
| (5 days) | ge4.1 | 38.0 | 6.3 |
| | wt | 0 | 0 |

### Example 2. Deletion (Skipping) of Dystrophin Exon 51

It was confirmed whether skipping of exon 51 was caused by the CRISPR/Cas12f1 (Cas14) system. A pair of gRNAs (ge4.0-F142 and ge4.0-R52, 3 ug each) and the Cas12f1 vector (5 ug) were transfected into AC16 cells and HEK293 cells in the same manner as Example 1.4. Four days after transfection, the cells were harvested, gDNA was extracted therefrom, and the fragments were identified by PCR. Next, the relative amount of exon 51, which remains undeleted, was quantified by qPCR using exon 51-targeting primers (LD1 and LD5). SaCas9 was used as a comparative control. The results are shown in Tables 13 (AC16 cells) and 14 (HEK293 cells).

TABLE 13

| | | |
|---|---|---|
| Cas12f1 | SaCas9 | No treatment |

LD1 Primer

| | | | |
|---|---|---|---|
| Deletion efficiency (%) of | 30.9 | 41.5 | 0 |
| exon 51 | | | |

LD5 Primer

| | | | |
|---|---|---|---|
| Deletion efficiency (%) of | 17.3 | 43.4 | 0 |
| exon 51 | | | |

TABLE 14

| | | |
|---|---|---|
| Cas12f1 | SaCas9 | No treatment |

LD1 Primer

| | | | |
|---|---|---|---|
| Deletion efficiency (%) of | 37.8 | 52.4 | 0 |
| exon 51 | | | |

LD5 Primer

| | | | |
|---|---|---|---|
| Deletion efficiency (%) of | 49 | 53.9 | 0 |
| exon 51 | | | |

Referring to Tables 13 and 14, it was found that deletion of exon 51 was caused by the system according to an embodiment of the present disclosure, and the rate was slightly lower in Cas12f1 than in SaCas9. In addition, in order to identify deletion efficiency overtime after transfecting the CRISPR/Cas12f1 system into cells, the SaCas9 system and the Cas12f1 system were transfected into AC16 cells and the deletion efficiency was checked on day 3, day 5, and day 7 after transfection. The primer used was LD1 primer, and the results are shown in Table 15.

TABLE 15

| | Day 3 | | | Day 5 | | | Day 7 | | |
|---|---|---|---|---|---|---|---|---|---|
| | | | No | | | No | | | No |
| | Cas12f1 | SaCas9 | treatment | Cas12f1 | SaCas9 | treatment | Cas12f1 | SaCas9 | treatment |
| Deletion | 28.0 | 55.0 | 0 | 37.1 | 55.6 | 0 | 56.2 | 60.8 | 0 |
| efficiency | | | | | | | | | |
| (%) of | | | | | | | | | |
| exon 51 | | | | | | | | | |

The CRISPR/Cas12f1 (Cas14) system showed increased large deletion efficiency of exon 51 over time after transfection (day 3: 28%, day 5: 37.1%, and day 7: 56.2%). In addition, Cas12f1 showed a greater increase in deletion efficiency than SaCas9. Since Cas12f1 has the characteristic of cleaving an outer part of the target sequence, it is thought that upon cleavage of the outer part of the target sequence, no changes occur in the target sequence, thereby allowing for additional cleavage, which can significantly increase the deletion efficiency over time. That is, this means that the Cas12f1 system can cause deletion of exon 51 with higher efficiency compared to other CRISPR systems that cleave the target sequence. This also applies to TnpB (CWCas12f1), which is also known to cleave an outer part of a target sequence (results not shown). Given that the effect of AAV, which can be used for delivery of the CRISPR/Cas12f1 system or the TaRGET system utilizing TnpB(CWCas12f1), lasts for about a month, it is expected that a greater increase in deletion efficiency can be obtained.

### Example 3. Deletion of Dystrophin Exon 51 Depending on Combination of Target Sequences

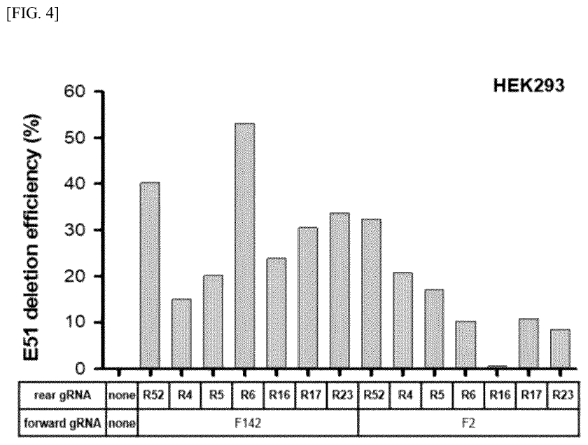

Deletion of exon 51 was confirmed in the same manner as in Example 1.4 using combinations of the spacer sequences that exhibited high indel efficiency in Example 1.3 (see Tables 8 and 9). The results of this experiment are shown in FIGS. 4 (HEK293 cells) and 5 (AC16 cells).

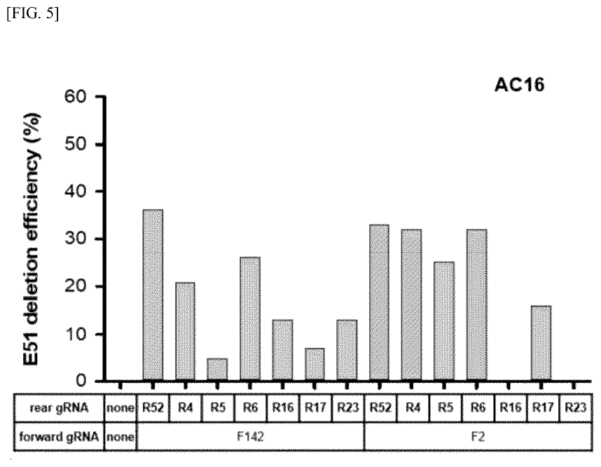

Referring to FIGS. 4 and 5, among the combinations of the spacer sequences, the combinations of F142/R52, F142/R6, and F2/R52 exhibited high deletion efficiency of exon 51.

### Example 4. Deletion of Dystrophin Exon 51 Depending on Promoter Type

To analyze the deletion efficiency of exon 51 by the CRISPR/Cas12f1 system or TaRGET system depending on promoter type, two gRNAs (upstream target ge4.0-F142 and downstream target ge4.0-R52) and either system (Cas12f1 or TaRGET system) were included in a single vector (one vector system), and large deletion of exon 51 caused thereby was checked depending on the promoter type.

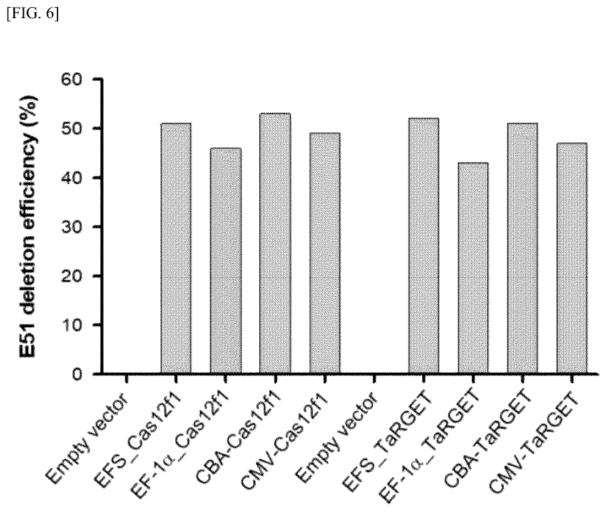

EFS (212 nt), EF-1-α (1182 nt), CMV (584 nt), and CBA (793 nt) were used as promoters, and the deletion efficiency of exon 51 was measured by the same method as in Example 1.4 using HEK293 cells and LD1 primer (FIG. 6).

Referring to FIG. 6, the Cas12f1 system showed the highest deletion efficiency of exon 51 in a case of using the CBA promoter (4th bar from the left in FIG. 6), while the TaRGET system showed high deletion efficiency of exon 51 in a case of using the EFS promoter that is the shortest and therefore is suitable for one vector system (4th bar from the right in FIG. 6).

It was confirmed that the Cas12f1 system or TaRGET system according to an embodiment of the present disclosure has a sufficiently small size to allow other elements to be further added even when included in a single vector (for example, AAV) system, given the size of TnpB (CWCas12f1) or Cas12f1 (UnCas12f1) and the length of the promoter. Accordingly, in the following examples, shRNA, which is considered as an additional element that can increase the deletion efficiency of exon 51, was introduced into the system to check deletion of exon 51.

### Example 5. Deletion of Exon 51 Through Inhibited Expression of Genes Involved in Non-Homologous End Joining (NHEJ) Repair Pathway

### Example 5.1. Selection of shRNA

In order to increase gene editing efficiency, expression of genes known to be involved in NHEJ repair pathway was inhibited using shRNA (short hairpin RNA).

Specifically, shRNAs (SEQ ID NOs: 360 to 389 and 403), which target each of six genes (ATM1, XRCC4, XLF-1, XRCC6, LIG4, and DCLRE1C) known to be involved in NHEJ, and a control scrambled shRNA (SEQ ID NO: 400) were produced. The shRNA molecules were produced in five types for each gene (six types of shRNA molecules for the ATM1 gene), and the specific sequence information thereof is shown in Table 16.

TABLE 16

| Gene | No | shRNA | SEQ ID NO |
|---|---|---|---|
| ATM1 | 1 | GGAGCCAGAUAGUUUGUAUUUCAAGAGAAUACAA | 360 |
| | | ACUAUCUGGCUCC | |
| | 2 | GCAAGCAGCUGAAACAAAUUUCAAGAGAAUUUGU | 361 |
| | | UUCAGCUGCUUGC | |
| | 3 | GGAGCUGAUUGUAGCAACAUUCAAGAGAUGUUGC | 362 |
| | | UACAAUCAGCUCC | |
| | 4 | GCACAGAAGUGCCUCCAAUUUCAAGAGAAUUGGA | 363 |
| | | GGCACUUCUGUGC | |
| | 5 | GGACAUAGUUUCUGGGAGAUUCAAGAGAUCUCCC | 364 |
| | | AGAAACUAUGUCC | |
| | 6 | GAACUUCAGUGGACCUUCAUUCAAGAGAUGAAGG | 403 |
| | | UCCACUGAAGUUC | |
| XRCC4 | 1 | GGAUGACACUGGCACAUUAUUCAAGAGAUAAUGU | 365 |
| | | GCCAGUGUCAUCC | |
| | 2 | GGAGAGUACUGAUGAGGAAUUCAAGAGAUUCCUC | 366 |
| | | AUCAGUACUCUCC | |
| | 3 | GAAUCCACCUUGUUUCUGAUUCAAGAGAUCAGAA | 367 |
| | | ACAAGGUGGAUUC | |
| | 4 | GUACAAGUAUCUUGGGAGAUUCAAGAGAUCUCCC | 368 |
| | | AAGAUACUUGUAC | |
| | 5 | GAAUGCAGCUCAAGAACGAUUCAAGAGAUCGUUC | 369 |
| | | UUGAGCUGCAUUC | |
| XLF-1 | 1 | GCAUGAGUCUGGCAUUACAUUCAAGAGAUGUAAU | 370 |
| | | GCCAGACUCAUGC | |
| | 2 | GAAAGCCCUUUGUCAUGAAUUCAAGAGAUUCAUG | 371 |
| | | ACAAAGGGCUUUC | |
| | 3 | GAACAGUGCUUCCCUGCAAUUCAAGAGAUUGCAG | 372 |
| | | GGAAGCACUGUUC | |
| | 4 | GGAAAGACCUAGAGAUCCAUUCAAGAGAUGGAUC | 373 |
| | | UCUAGGUCUUUCC | |
| | 5 | GUAUGGCAGUCACCACACAUUCAAGAGAUGUGUG | 374 |
| | | GUGACUGCCAUAC | |
| XRCC6 | 1 | GCAGCAUUGUGCAGAUACAUUCAAGAGAUGUAUC | 375 |
| | | UGCACAAUGCUGC | |
| | 2 | GCAGGAACAUCCCUCCUUAUUCAAGAGAUAAGGA | 376 |
| | | GGGAUGUUCCUGC | |
| | 3 | GCAGUGCUCUGCUCAUCAAUUCAAGAGAUUGAUG | 377 |
| | | AGCAGAGCACUGC | |
| | 4 | GGAUCAUGCUGUUCACCAAUUCAAGAGAUUGGUG | 378 |
| | | AACAGCAUGAUCC | |
| | 5 | GGAUCUGACUACUCACUCAUUCAAGAGAUGAGUG | 379 |
| | | AGUAGUCAGAUCC | |
| LIG4 | 1 | GCACAAAGAUGGAGAUGUAUUCAAGAGAUACAUC | 380 |
| | | UCCAUCUUUGUGC | |
| | 2 | GCAGACACGUACUGUGUAAUUCAAGAGAUUACAC | 381 |
| | | AGUACGUGUCUGC | |
| | 3 | GGAGCAGACUCCUGAAGAAUUCAAGAGAUUCUUC | 382 |
| | | AGGAGUCUGCUCC | |
| | 4 | GGAGGAUUCUGAUCUGCAAUUCAAGAGAUUGCAG | 383 |
| | | AUCAGAAUCCUCC | |
| | 5 | GCAUGAUCCUUCUGUAGGAUUCAAGAGAUCCUAC | 384 |
| | | AGAAGGAUCAUGC | |
| DCLREIC | 1 | GGAGACUCCUACCCAGAUAUUCAAGAGAUAUCUG | 385 |
| | | GGUAGGAGUCUCC | |
| | 2 | GGACAAAGCUGACUACAGAUUCAAGAGAUCUGUA | 386 |
| | | GUCAGCUUUGUCC | |
| | 3 | GCAGAGCUCUCGUUUCACAUUCAAGAGAUGUGAA | 387 |
| | | ACGAGAGCUCUGC | |
| | 4 | GGACUCUGAUGGAGAAUCAUUCAAGAGAUGAUUC | 388 |
| | | UCCAUCAGAGUCC | |
| | 5 | GCAGAAUUCUUCCCAGUCAUUCAAGAGAUGACUG | 389 |
| | | GGAAGAAUUCUGC | |
| scrambled | 3 | CAGAGCUAACUCAGAUAGUACU | 400 |

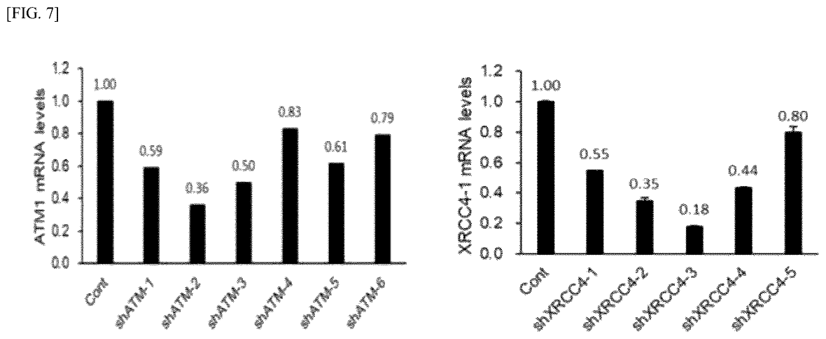

shRNA was transfected into previously prepared AC16 cells at a dose of 5 ug. After incubation for 3 days, the cells were harvested and the mRNA expression levels of the genes were measured using qRT-PCR. The results are shown in FIGS. 7 (ATM1 and XRCC4), 8 (XLF-1 and XRCC6), and 9 (LIG4 and DCLRE1C).

### Example 5.2. Deletion of Exon 51 by System Comprising shRNA

Measurement of Deletion Efficiency of Exon 51 Using One shRNA

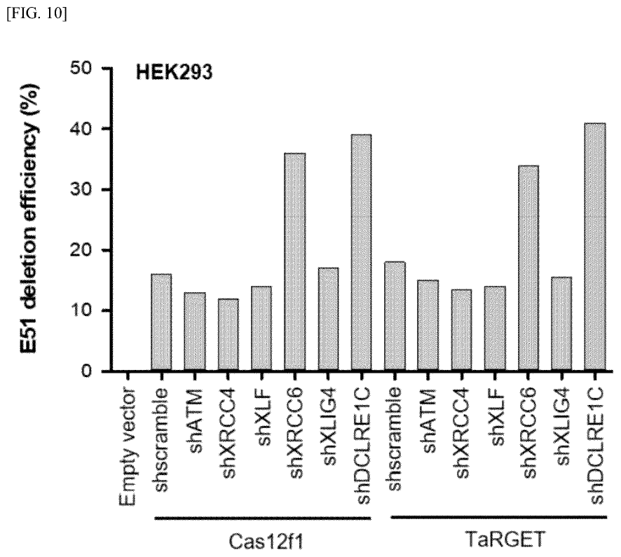

Based on the qRT-PCR results, for each gene, the shRNA with the highest inhibition efficiency for mRNA expression was selected. Nucleic acids encoding the shRNA, two guide RNAs, and TnpB (CWCas12f1) or Cas12f1 were inserted into a single vector, and transfected into HEK293 cells and AC16 cells using the same method as Example 1.4 to measure the relative deletion efficiency of exon 51. Here, relative means a relative value under the same conditions, as indel efficiency varies depending on the transfection time, vector type, and concentration. The results are shown in FIGS. 10 (HEK293 cells) and 11 (AC16 cells).

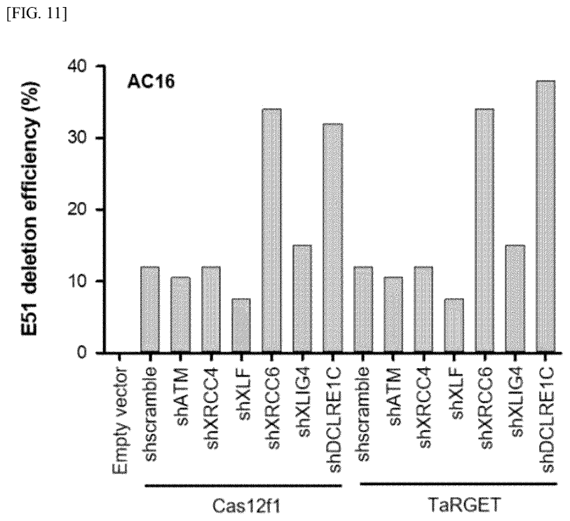

Referring to FIGS. 10 and 11, it was confirmed that exon 51 was effectively deleted through inhibited expression of NHEJ-related genes in both TnpB and Cas12f1 systems. In particular, higher deletion levels of exon 51 were observed in a case of using shXRCC6 and shDCLRE1C.

Measurement of Deletion Efficiency of Exon 51 Using Two or More shRNAs

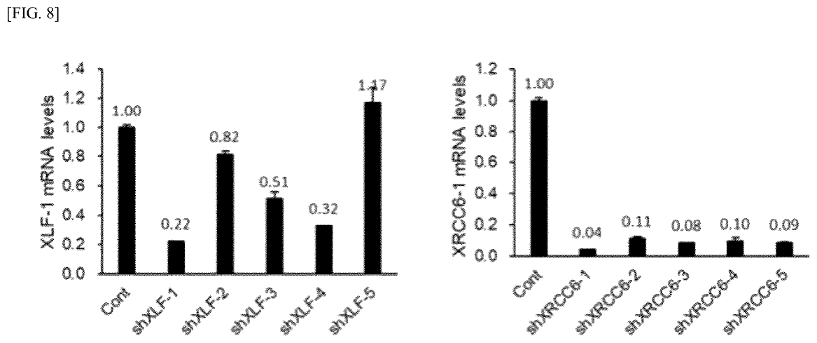

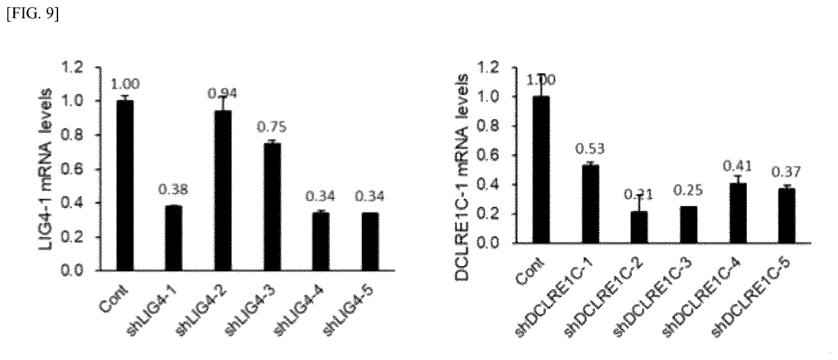

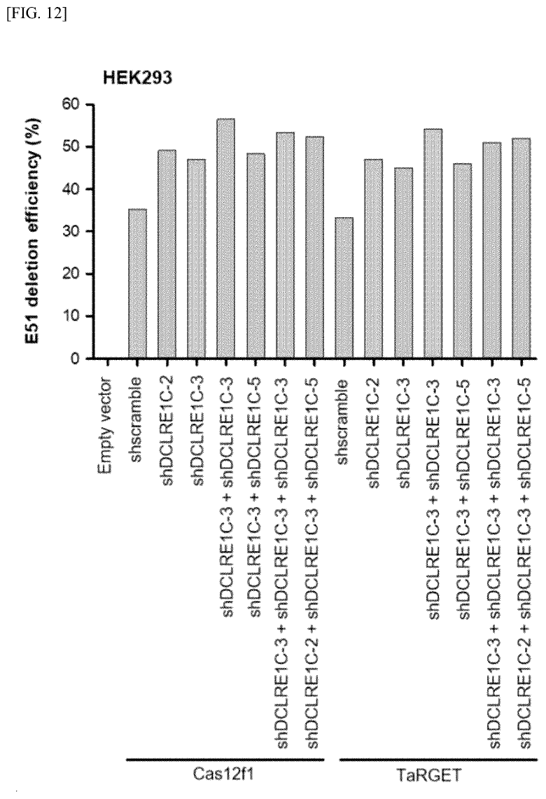

Deletion of exon 51 was induced using two or more identical or different shRNAs. Nucleotide sequences encoding two guide RNAs, TnpB or Cas12f1, and shDCLRE1C (one, two, or three selected from shDCLRE1C2, shDCLRE1C3, and shDCLRE1C5) were inserted into a single vector and transfected into HEK293 cells and AC16 cells in the same manner as in Example 1.4 to measure the relative deletion efficiency of exon 51. Here, for the shDCLRE1C used, among the five shRNAs, the three with the best inhibition efficiency for mRNA expression were selected (right one in FIG. 8). The results are shown in FIGS. 12 (HEK293 cells) and 13 (AC16 cells).

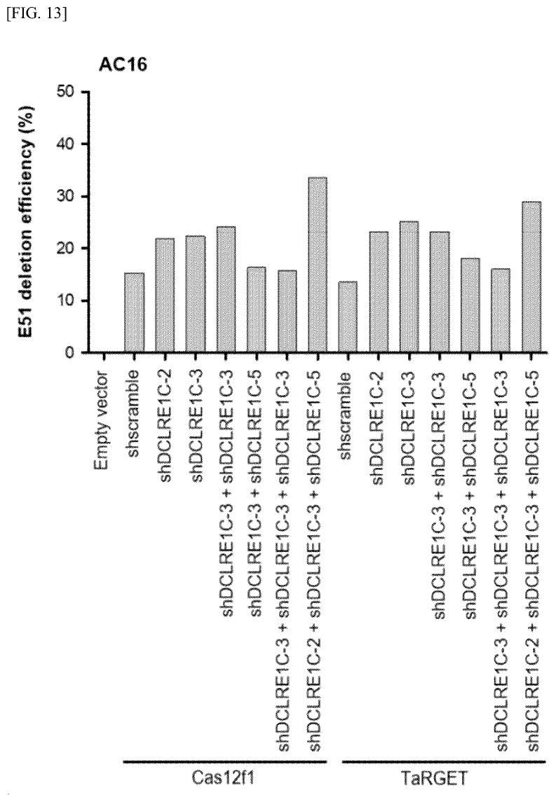

Referring to FIGS. 12 and 13, deletion of exon 51 occurred well in all experimental groups, and the deletion efficiency of exon 51 was particularly excellent when two or more shDCLRE1Cs were introduced.

### Example 5.3. Deletion of Exon 51 Over Time after Transfection with System Comprising shRNA

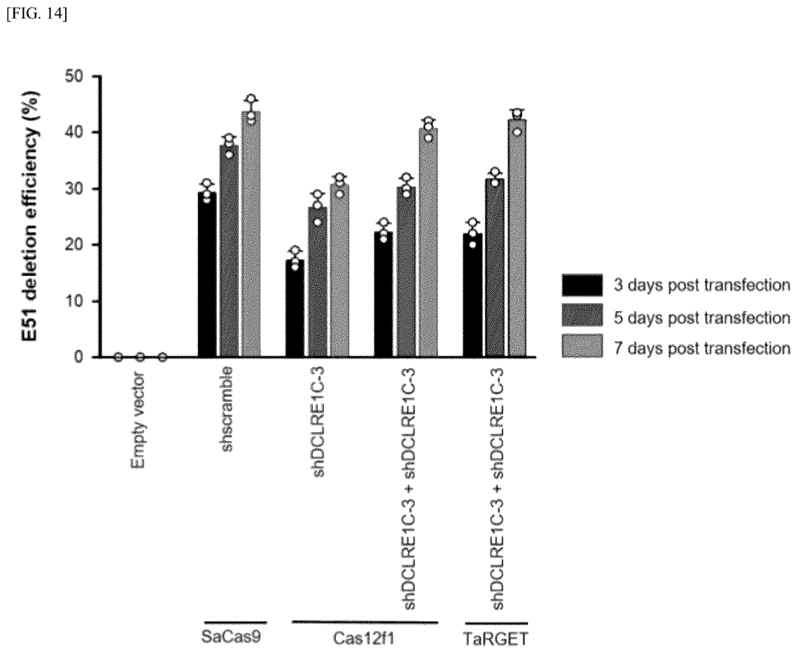

To determine the deletion efficiency of exon 51 depending on different transfection periods (after 3 days, 5 days, and 7 days) using one or more shRNAs, nucleic acids encoding two guide RNAs, TnpB or Cas12f1, and one or more shRNAs were inserted into one vector, and then the deletion efficiency of exon 51 was determined over time starting from the day of transfection. For all experiments, AC16 cells were used, and the empty vector and SaCas9 system were prepared as controls for comparison. The results are shown in FIG. 14.

Referring to FIG. 14, the deletion efficiency of exon 51 increased over time starting from the day of transfection in all experimental groups; and in particular, the Cas12f1 and TaRGET systems, in which a combination of two shDCLRE1Cs are used, showed similar deletion efficiency of exon 51 to that of SaCas9 after 7 days of transfection.

### Example 5.4. Confirmation of Deletion of Exon 51 Depending on Promoter Type in System Comprising shRNA

For vectors comprising nucleic acids that encode two guide RNAs, TnpB or Cas12f1, and one or more shRNAs, the deletion efficiency of exon 51 was checked through qRT-PCR depending on the promoter type.

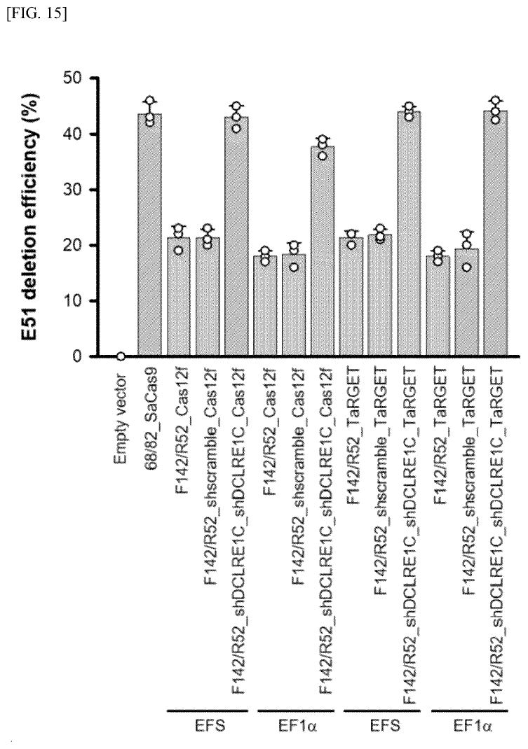

Referring to FIG. 15, when using two shDCLRE1Cs, both TaRGET and Cas12f1 systems showed high deletion efficiency of exon 51 regardless of the promoter type (EFS and EF1α), and the efficiency was similar to that of the SaCas9 system.

### CONCLUSION

As such, the CRISPR/Cas12f1 and TaRGET systems of the present disclosure, which comprise two guide RNAs with optimized spacer sequences and TnpB or Cas12f1 recognizing the target sequence, can cleave the target regions upstream and downstream of exon 51 in the dystrophin gene, thereby inducing production of normal dystrophin protein due to deletion of the exon (exon skipping strategy). In addition, the deletion efficiency of exon 51 in the dystrophin gene can be further increased by additionally using shRNA that inhibits expression of a protein involved in the NHEJ pathway.

### Experimental Method

1. Extraction of gDNA

Extraction of gDNA was performed using the Genomic DNA Prep Kit (Maxwell® RSC Tissue DNA Kit, Promega). The medium of the transfected cells was removed from the 24-well plate, and 200 μl of trypsin was added to each well to detach the cells from the bottom. Then, the cells were transferred to a 1.5 ml tube. The tube was centrifuged at 300×g for 5 minutes and the supernatant was removed. 200 μl of PBS was added to the tube containing the cells for resuspension. Then, the respective cells were transferred to well #1 of the Maxwell cartridge. A plunger was inserted into well #8 of the cartridge. An empty elution tube was placed in each position of the deck tray. 100 μl of elution buffer was added to the elution tube. The prepared deck tray was installed in the Maxwell machine and set up. Then, the machine was operated. After the extraction process was completed, the eluted gDNA was quantified and stored at 4° C.

### 2. PCR and Gel Purification

This experiment was performed using the GEL & PCR Purification System (GP104-200, Biofact). To the PCR product was added UB buffer in an amount equivalent to 3 times the volume of the PCR product and thorough mixing was performed. Then, isopropanol was added thereto in an amount equivalent to 2 times the volume of the PCR product and thorough mixing was performed. In a case of the gel, the gel of the corresponding band was cut and weighed. Then, UB buffer was added thereto in an amount equivalent to 3 times the weight of the gel. The gel was dissolved by incubation at 65° C. for 10 minutes. Then, isopropanol was added thereto in an amount equivalent to 1 time the gel volume and thorough mixing was performed. The column was prepared, 200 μl of HelpB buffer was added to the column, and centrifugation was performed at 13,000 rpm for 30 seconds. Then, the filtered solution was discarded. The reaction solution was added to the column, and centrifugation was performed at 7,000 rpm for 1 minute. Then, the filtered solution was discarded. 750 μl of 80% EtOH was added thereto, and centrifugation was performed at 13,000 rpm for 30 seconds. Then, the filtered solution was discarded. After repeating the process twice, centrifugation was performed at 13,000 rpm for 3 minutes. The centrifuged column was placed in a 1.5 ml tube, 30 μl of EB buffer was added dropwise to the center, and the reaction was allowed to occur at room temperature for 1 minute. Centrifugation was performed at 13,000 rpm for 1 minute. The DNA collected in the 1.5 ml tube was quantified and then stored at 4° C.

### 3. Collection of Plasmid Vector

For transfection or Sanger sequencing, the vector-transformed DH5α was used. Plasmid Mini prep kit (PM105-200, Biofact) was used according to the manufacturer's instructions. The culture medium of the vector-transformed DH5α was placed in a 1.5 ml tube, and centrifugation was performed at 13,000 rpm for 5 minutes. After centrifugation, the supernatant was discarded, and the pellet was sufficiently dispersed by vortexing. 350 μl of Bi buffer was added thereto, and the tube was shaken to ensure sufficient reaction. Next, 350 μl of A1 buffer containing RNase A was added thereto, and the tube was inverted until the blue color disappeared. Then, centrifugation was performed at 13,000 rpm for 5 minutes. The column was prepared, 200 μl of HelpB buffer was added thereto, and centrifugation was performed at 13,000 rpm for 30 seconds. Then, the filtered solution was discarded. 750 μl of the centrifuged supernatant was added to the prepared column, centrifugation was performed at 7,000 rpm for 1 minute, and the filtered solution was discarded. 750 μl of 80% EtOH was added thereto, centrifugation was performed at 13,000 rpm for 30 seconds, and then the filtered solution was discarded. This process was repeated twice. After repeating the process twice, centrifugation was performed at 13,000 rpm for 3 minutes. The centrifuged column was placed into a 1.5 ml tube, 30 μl of EB buffer was added dropwise to the center, and then the reaction was allowed to occur at room temperature for 1 minute. Centrifugation was performed at 13,000 rpm for 1 minute. The plasmid vectors collected in the 1.5 ml tube were quantified and then stored at −20° C.

### 4. Preparation of DNA Cassette

To check indel efficiency of the spacer sequences of Cas12f1, a cassette containing the U6 promoter, scaffold sequence, guide sequence, and U-rich tail sequence (T₄AT₆) was amplified by PCR and used. The process was performed as follows.

### 1) Selection of Spacer and Order of Oligo

The spacer was selected from the 20-mer sequences following TTTA or TTTG, which is PAM, and spacers whose sequences end with T were excluded. In addition, to decrease off-target effects, the spacers were designed using CRISPR RGEN TOOL by classifying them with less than 2 mismatches. In addition, a reverse complement sequence comprising DR (direct repeat) and U-rich sequence was custom-made to be used as an R primer.

### 2) PCR

The PCR was performed under the composition and condition shown in Table 17 below.

TABLE 17

| Reagent composition | | | PCR condition | |
|---|---|---|---|---|
| 2x pfu PCR | 200 | μl | Pre-denaturation | 95° C., 5 min |
| Master mix | | | | |
| hU6 F primer | 2 | μl | Denaturation, D | 95° C., 30 s |
| (10 P) | | | | |
| Target oligo | 2 | μl | Annealing | 58° C., 40 s |
| (10 P) | | | | |
| Template | 1 | μl (200 ng) | Extension, E | 72° C., 40 s |
| DW | 195 | μl | D-E Cycle | 35 cycles |
| Total | 400 | μl | Final extension | 72° C., 3 min |

| | | |
|---|---|---|
| Prepared in 4 PCR tubes, each | Storage | 4° C., ∞ |
| containing 100 μl each | | |

400 μl of the mixture was added to 4 PCR tubes, each containing 100 μl, and each sample was amplified.

### 3) Gel Analysis

2% agarose gel was prepared, and the size marker and PCR products were added to the gel. Electrophoresis was performed to check the amplified size.

### 4) Purification and Quantification

After checking the amplified size, the gel was purified according to Experimental Method 2 to quantify the PCR products.

### 5. Cell Culture

HEK293 cells and AC16 cells used in the experiment were cultured using DMEM (containing 10% FBS, 1% penicillin-streptomycin) medium. The frozen cells were quickly thawed at 37° C., placed in 5 ml of pre-warmed cell medium, and thoroughly dissolved. Then, centrifugation was performed at 1,500 rpm for 3 minutes. After centrifugation, the supernatant containing the remaining cryostat was quickly removed, the pellet was thoroughly resuspended in cell medium, and then divided into two 90 mm dishes for culture, each containing 10 ml of medium. The next day, the medium was replaced with new medium, and subculture was performed when the cell confluency reached 80%. Here, HEK293 was subcultured at a ratio of 1/5, and AC16 was subcultured at a ratio of 1/4.

### 6. Transfection

The day before transfection, HEK293 and AC16 cells (80% confluency) cultured in 100 mm dishes were treated with trypsin to detach the cells from the bottom of the dish. The detached cells were placed in 50 ml of pre-warmed medium and slowly resuspended with a pipette. 24-well plates were prepared according to the number of samples and repetitions, and 500 μl of cell suspension medium was added to each well (1/100 dilution). Then, incubation was performed overnight in a CO₂ incubator at 37° C. until transfection.

When the cell confluency reached approximately 70% to 80%, 200 μl out of the 500 μl medium per well was removed and the plates were placed in the incubator. 1.5 ml tubes were prepared according to the number of samples, and 200 μl of Opti-MEM was added to each tube. 1.5 μg of Cas12f1 and 0.5 μg of gRNA were added to the tube containing Opti-MEM, and vortexed for 5 seconds (nucleic acid mixture). Then, the nucleic acid mixture and FuGENE HD were added at a ratio of 1:3, and reaction was allowed to occur at room temperature for 20 minutes (that is, in a case where the nucleic acid mixture was 2 μg, 6 μl of FuGENE HD was added). The 24-well plate was taken out from the incubator, and 200 μl of the solution containing the nucleic acid mixture and FuGENE HD was gently added along the well wall. After shaking the plate sufficiently in an S shape, incubation was performed in a CO₂ incubator at 37° C. for 72 hours. After 72 hours, the cells were harvested and gDNA was extracted therefrom according to Experimental Method 1.

### 7. Construction of Vector

The following procedure was performed using the Cas12f1 ge4.0 dual gRNA vector (see Korean Patent Application Nos. 10-2021-0051552 and 10-2022-0043768). The restriction enzyme ends of the vector to be cloned were checked, and dual gRNA oligos were designed and custom-made. The custom-made oligos were diluted to a concentration of 100 pmol. 4.5 μl each of the diluted forward and reverse primers were taken and put into a PCR tube. Then, 1 μl of 10× annealing buffer was added thereto to adjust the total volume to 10 μl. Then, annealing was performed under the conditions of 95° C. for 5 minutes and −1° C./min from 95° C. to 4° C. The Cas12f1 ge4.0 dual gRNA vector was prepared and digested at 500 rpm and 37° C. for 2 hours under the conditions for digestion in Table 18 below.

TABLE 18

| Reagent | Volume | |
|---|---|---|
| NEB 10X rCutsmart buffer | 5 | μl |
| Vector | 10 | μg |
| BbsI-HF | 1 | μl |
| DW | Amount to make total volume of 50 μl | |
| Total | 50 | μl |

After digestion, the digested vector was obtained through electrophoresis and gel elution. Ligation was performed using the digested vector and annealed oligo (see Table 19).

TABLE 19

| Reagent | Volume | |
|---|---|---|
| 2X Rapid ligation buffer | 2.5 | μl |
| T4 DNA ligase (Promega) | 0.5 | μl |
| Annealed oligo | 1.5 | μg |
| Vector digested with BbsI | 0.5 | μl |
| Total | 5 | μl |

After ligation, transformation was performed on DH5α. After incubation on an LB plate, positive colonies were checked through colony PCR and then incubated in 3 ml LB medium. After miniprep, sequencing was performed to confirm whether the final sequences matched.

### 8. DH5α Transformation

The previously-produced vector was transformed into E. coli to produce the vector. DH5α competent cells were taken out and thawed on ice. The ligated vector was added up to 1/10 of the amount of DH5α, and then incubation was performed on ice for 30 minutes. After heat shock at 42° C. for 30 seconds, cooling was performed on ice for 2 minutes. Incubation was performed using 100 μl of LB medium or S.O.C medium at 37° C. for 1 hour. The cells were spread on LB plates warmed to room temperature (containing ampicillin or kanamycin depending on the vector) and incubated at 37° C. for 14 to 16 hours.

### 9. PCR of NGS Sample

A total of 3 PCRs were performed to check the indel efficiency. The first PCR produced a band of approximately 450 to 500 bp, and the second PCR was performed using this PCR product as a template. After the second PCR, the product was loaded onto a 2% agarose gel to check whether the band was properly displayed within 250 bp. If the band was not properly displayed, the cause was determined. Then, the process was restarted from the first PCR. If the correct band was confirmed, the third PCR was performed using the second PCR product as a template. Here, if the concentration of the second PCR product was high, DW was added to adjust the concentration. After completing the third PCR, the sample was loaded onto a 2% agarose gel to identify the bands. The completed PCR products were pooled in equal amounts (5 μl each) and then subjected to PCR purification.

PCR purification was performed using the GEL & PCR Purification System (GP104-200, Biofact). UB buffer was added to the PCR product in an amount equivalent to 5 times the volume of the PCR product and thorough mixing was performed. The column was prepared, and 200 μl of HelpB buffer was added to the column. Then, centrifugation was performed at 13,000 rpm for 30 seconds, and the filtered solution was discarded. The reaction solution was added to the column, and centrifugation was performed at 7,000 rpm for 1 minute. Then, the filtered solution was discarded. 750 μl of 80% EtOH was added thereto, and centrifugation was performed at 13,000 rpm for 30 seconds. Then, the filtered solution was discarded. After repeating the process twice, centrifugation was performed at 13,000 rpm for 3 minutes. The centrifuged column was placed in a 1.5 ml tube, 100 μl of EB buffer was added dropwise to the center, and then the reaction was allowed to occur at room temperature for 1 minute. Centrifugation was performed at 13,000 rpm for 1 minute. The DNA collected in the 1.5 ml tube was quantified to adjust the concentration to 15 ng/μl and stored at 4° C. until NGS analysis.

TABLE 20

| Reagent composition | | | PCR condition | |
|---|---|---|---|---|
| SUN PCR blend mix | 5 | μl | Pre-denaturation | 95° C., 3 min |
| Forward primer | 0.5 | μl | Denaturation (D) | 98° C., 20 s |
| (10 pmol/ul) | | | | |
| Reverse primer | 0.5 | μl | Annealing (A) | 60° C., 30 s |
| (10 pmol/ul) | | | | |
| Template (gDNA) | 1 | μl | Extension (E) | 72° C., 30 s |
| DW | 3 | μl | D-E Cycle | 30 cycles |
| Total | 10 | μl | Final extension | 72° C., 3 min |

| | |
|---|---|
| Storage | 4° C., ∞ |

1-130. (canceled)

131. An editing system for a dystrophin gene, comprising:
an endonuclease comprising Cas12f1 or a variant protein thereof, or a nucleic acid encoding the endonuclease;
an engineered guide RNA comprising a first guide sequence that hybridizes to a target sequence in a dystrophin gene, or a nucleic acid encoding the guide RNA; and
an engineered guide RNA comprising a second guide sequence that hybridizes to a target sequence in a dystrophin gene, or a nucleic acid encoding the guide RNA,
wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and
the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

132. The system of claim 131, wherein the system is applied to a cell to cause deletion of dystrophin exon 51.

133. The system of claim 131, wherein the system is for treatment of Duchenne muscular dystrophy.

134. The system of claim 131, wherein the first guide sequence is a sequence hybridizable to a target sequence that is complementary to a nucleotide sequence selected from the group consisting of SEQ ID NOs: 190 to 217 and SEQ ID NOs: 255 to 280, wherein the nucleotide sequence is located in a non-target strand of a region 5000 bp upstream of dystrophin exon 51, and
the second guide sequence is a sequence hybridizable to a target sequence that is complementary to a nucleotide sequence selected from the group consisting of SEQ ID NOs: 218 to 254 and SEQ ID NOs: 281 to 311, wherein the nucleotide sequence is located in a non-target strand of a region 5000 bp downstream of dystrophin exon 51.

135. The system of claim 134, wherein the first guide sequence comprises a sequence of contiguous 15 to 20 nucleotides from a nucleotide sequence selected from the group consisting of SEQ ID NOs: 190 to 217 and SEQ ID NOs: 255 to 280, wherein thymine (T) in the contiguous nucleotide sequence is substituted with uracil (U) and/or,
the second guide sequence comprises a sequence of contiguous 15 to 20 nucleotides from a nucleotide sequence selected from the group consisting of SEQ ID NOs: 218 to 254 and SEQ ID NOs: 281 to 311, wherein thymine (T) in the contiguous nucleotide sequence is substituted with uracil (U).

136. The system of claim 135, wherein the first guide sequence comprises a nucleotide sequence selected from the group consisting of SEQ ID NOs: 312 to 323 and/or,
the second guide sequence comprises a nucleotide sequence selected from the group consisting of SEQ ID NOs: 324 to 335.

137. The system of claim 131, wherein the engineered guide RNA comprises a U-rich tail sequence linked to the 3′-end of the first or second guide sequence, in which the U-rich tail is represented by 5′-(UₘV)ₙUₒ-3′, wherein V is each independently A, C, or G, m and o are integers between 1 and 20, and n is an integer between 0 and 5.

138. The system of claim 131, wherein the engineered guide RNA comprises a nucleotide sequence having at least 50% sequence identity to a scaffold region of a wild-type Cas12f1 guide RNA sequence, in which the scaffold region of the wild-type Cas12f1 guide RNA sequence sequentially comprises, from the 5′-end, a first stem-loop region, a second stem-loop region, a third stem-loop region, a fourth stem-loop region, and a tracrRNA-crRNA complementarity region, and
the engineered guide RNA comprises at least one modification selected from the group consisting of the following (1) to (5) with respect to the wild-type Cas12f1 guide RNA sequence:
(1) deletion of at least a part of the first stem-loop region;
(2) deletion of at least a part of the second stem-loop region;
(3) deletion of at least a part of the tracrRNA-crRNA complementarity region;
(4) replacement of one or more uracil (U) residues with A, G, or C in three or more consecutive U residues when the consecutive U residues are present in the tracrRNA-crRNA complementarity region; and
(5) addition of a U-rich tail to the 3′-end of the crRNA sequence (in which a sequence of the U-rich tail is represented by 5′-(UₘV)ₙUₒ-3′, wherein V is each independently A, C, or G, m and o are integers between 1 and 20, and n is an integer between 0 and 5).

139. The system of claim 138, wherein the wild-type Cas12f1 guide RNA comprises tracrRNA comprising the nucleotide sequence of SEQ ID NO: 11 and crRNA comprising the nucleotide sequence of SEQ ID NO: 12.

140. The system of claim 138, wherein the engineered guide RNA comprises at least one modification selected from (5) addition of a U-rich tail to the 3′-end of the crRNA sequence and (4) replacement of one or more uracil (U) residues with A, G, or C in three or more consecutive U residues when the consecutive U residues are present in the tracrRNA-crRNA complementarity region.

141. The system of claim 138, wherein the engineered guide RNA comprises at least one modification selected from (1) deletion of at least a part of the first stem-loop region; (2) deletion of at least a part of the second stem-loop region; and (3) deletion of at least a part of the tracrRNA-crRNA complementarity region.

142. The system of claim 138, wherein the engineered guide RNA comprises (3) deletion of a part of the tracrRNA-crRNA complementarity region, wherein the part of the complementarity region consists of 1 to 54 nucleotides, or (3) deletion of the entire tracrRNA-crRNA complementarity region, wherein the entire complementarity region consists of 55 nucleotides.

143. The system of claim 138, wherein the engineered guide RNA comprises (1) deletion of at least a part of the first stem-loop region, wherein the at least a part of the stem-loop region consists of 1 to 20 nucleotides.

144. The system of claim 138, wherein the engineered guide RNA comprises (2) deletion of at least a part of the second stem-loop region, wherein the at least a part of the stem-loop region consists of 1 to 27 nucleotides.

145. The system of claim 138, wherein the engineered guide RNA comprises at least one modification selected from (1) deletion of at least a part of the first stem-loop region; and (5) addition of a U-rich tail to the 3′-end of the crRNA sequence.

146. The system of claim 131, wherein the engineered guide RNA consists of a sequence represented by following Formula (I) or has at least 80% sequence identity to the sequence: 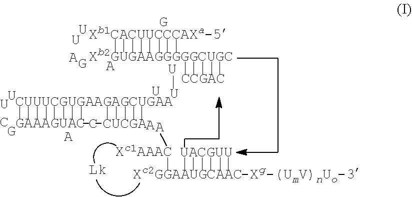
in Formula (I),
X^{a}, X^{b1}, X^{b2}, X^{c1}, and X^{c2} each independently consist of 0 to 35 (poly)nucleotides,
X^{g} is the first or second guide sequence,
Lk is a polynucleotide linker of 2 to 20 nucleotides in length or is absent, and
(UₘV)ₙUₒ is a U-rich tail and is present or absent, and in a case where the U-rich tail is present, U is uridine, V is each independently A, C or G, m and o are each independently an integer between 1 and 20, and n is an integer between 0 and 5.

147. The system of claim 146, wherein X^{a} comprises the nucleotide sequence of SEQ ID NO: 14 or a deleted form of the sequence of SEQ ID NO: 14 with 1 to 20 nucleotides deleted therefrom, or
wherein X^{b1} comprises the nucleotide sequence of SEQ ID NO: 25 or a deleted form of the sequence of SEQ ID NO: 25 with 1 to 13 nucleotides deleted therefrom, or
wherein X^{b2} comprises the nucleotide sequence of SEQ ID NO: 29 or a deleted form of the sequence of SEQ ID NO: 29 with 1 to 14 nucleotides deleted therefrom.

148. The system of claim 146, wherein the sequence 5′-X^{b1}UUAGX^{b2}-3′ in Formula (I) is a nucleotide sequence selected from the group consisting of SEQ ID NOs: 34 to 38, and UUAG.

149. The system of claim 146, wherein X^{c1} comprises the nucleotide sequence of SEQ ID NO: 39 or a deleted form of the sequence of SEQ ID NO: 39 with 1 to 28 nucleotides deleted therefrom.

150. The system of claim 149, wherein in a case where three or more consecutive uracil (U) residues are present in a sequence of X^{c1}, the sequence of X^{c1} comprises a modification in which at least one U residue thereof is replaced with A, G, or C.

151. The system of claim 146, wherein X^{c2} comprises the nucleotide sequence of SEQ ID NO: 58 or a deleted form of the sequence of SEQ ID NO: 58 with 1 to 27 nucleotides deleted therefrom.

152. The system of claim 151, wherein in a case where the sequence 5′-ACGAA-3′ is present in X^{c2}, the sequence is replaced with 5′-NGNNN-3′, wherein N is each independently A, C, G, or U.

153. The system of claim 146, wherein the sequence 5′-X^{c1}-Lk-X^{c2}-3′ in Formula (I) is a nucleotide sequence selected from the group consisting of SEQ ID NOs: 80 to 86, or is 5′-Lk-3′.

154. The system of claim 146, wherein Lk comprises a nucleotide sequence selected from the group consisting of 5′-GAAA-3′, 5′-UUAG-3′, 5′-UGAAAA-3′, 5′-UUGAAAAA-3′, 5′-UUCGAAAGAA-3′ (SEQ ID NO: 76), 5′-UUCAGAAAUGAA-3′ (SEQ ID NO: 77), 5′-UUCAUGAAAAUGAA-3′ (SEQ ID NO: 78), and 5′-UUCAUUGAAAAAUGAA-3′ (SEQ ID NO: 79).

155. The system of claim 146, wherein (UₘV)ₙUₒ is such that (i) n is 0 and o is an integer between 1 and 6, or (ii) V is A or G, m and o are each independently an integer between 3 and 6, and n is an integer between 1 and 3.

156. The system of claim 131, wherein the engineered guide RNA comprises an engineered tracrRNA consisting of a nucleotide sequence selected from the group consisting of SEQ ID NOs: 87 to 132.

157. The system of claim 131, wherein the engineered guide RNA comprises an engineered crRNA, wherein the engineered crRNA comprises a nucleotide sequence selected from the group consisting of SEQ ID NOs: 133 to 148.

158. The system of claim 131, wherein the engineered guide RNA is a dual guide RNA or a single guide RNA.

159. The system of claim 158, wherein the engineered single guide RNA consists of a nucleotide sequence selected from the group consisting of SEQ ID NOs: 149 to 186.

160. The system of claim 131, wherein the Cas12f1 or variant protein thereof comprises an amino acid sequence having at least 70% sequence identity to an amino acid sequence selected from the group consisting of SEQ ID NOs: 1 to 5.

161. The system of claim 131, wherein the endonuclease comprising Cas12f1 or a variant protein thereof, and the engineered guide RNA comprising a first guide sequence or the engineered guide RNA comprising a second guide sequence are in the form of a ribonucleoprotein (RNP).

162. The system of claim 131, wherein the system further comprises a molecule that inhibits expression of a gene involved in non-homologous end joining (NHEJ) or a nucleic acid encoding the molecule.

163. The system of claim 162, wherein the gene involved in NHEJ is at least one selected from the group consisting of ATM1, XRCC4, XLF, XRCC6, LIG4, and DCLRE1C.

164. The system of claim 162, wherein the gene involved in NHEJ is at least one selected from the group consisting of XRCC6 and DCLRE1C.

165. The system of claim 162, wherein the molecule is shRNA, siRNA, miRNA, or an antisense oligonucleotide.

166. The system of claim 165, wherein the shRNA molecule is at least one selected from the group consisting of shXRCC6 and shDCLRE1C.

167. The system of claim 163, wherein the shRNA molecule is at least one selected from the group consisting of SEQ ID NOs: 360 to 389 and 403.

168. A vector system, comprising at least one vector that comprises:
a first nucleic acid construct to which a nucleotide sequence encoding an endonuclease is operably linked, the endonuclease comprising Cas12f1 or a variant protein thereof,
a second nucleic acid construct to which an engineered guide RNA or a nucleotide sequence encoding the engineered guide RNA is operably linked, the engineered guide RNA comprising a first guide sequence that hybridizes to a target sequence in a dystrophin gene; and
a third nucleic acid construct to which an engineered guide RNA or a nucleotide sequence encoding the engineered guide RNA is operably linked, the engineered guide RNA comprising a second guide sequence that hybridizes to a target sequence in a dystrophin gene
wherein the first guide sequence is capable of hybridizing to a target sequence of contiguous 15 to 30 bp in length, wherein the target sequence is adjacent to the 5′-end or the 3′-end of a protospacer-adjacent motif (PAM) sequence present in a region 5000 bp upstream of dystrophin exon 51, and
the second guide sequence is capable of hybridizing to a target sequence of contiguous 15 to 30 bp in length, wherein the target sequence is adjacent to the 5′-end or the 3′-end of a PAM sequence present in a region 5000 bp downstream of dystrophin exon 51.

169. The vector system of claim 168, wherein the vector is at least one viral vector selected from the group consisting of a retroviral (retrovirus) vector, a lentiviral (lentivirus) vector, an adenoviral (adenovirus) vector, an adeno-associated viral (adeno-associated virus) vector, a vaccinia viral (vaccinia virus) vector, a poxviral (poxvirus) vector, a herpes simplex viral (herpes simplex virus) vector, and a phagemid vector.

170. An engineered guide RNA, comprising a spacer region, which comprises a guide sequence capable of hybridizing to a target sequence in a dystrophin gene, and a scaffold region,
wherein the guide sequence is capable of hybridizing to a target sequence of contiguous 15 to 30 bp in length, wherein the target sequence is adjacent to the 5′-end or the 3′-end of a protospacer-adjacent motif (PAM) sequence which is present in a region 5000 bp upstream or downstream of dystrophin exon 51 and is recognized by Cas12f1 or a variant protein thereof.

171. A method for deleting a segment comprising exon 51 in a dystrophin gene in a cell, comprising bringing into contact with the cell the system of claim 131.


SEQUENCE LISTING (403 sequences)

SEQ ID NO: 1 - protein, 557 aa, unidentified
  source 1..557: note = TnpB
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 2 - protein, 555 aa, synthetic construct
  source 1..555: note = TnpB-v1
MEKRINKIRKKLSADNATKPVSRSGPMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 3 - protein, 557 aa, synthetic construct
  source 1..557: note = TnpB-v2
MAGGPGAGSAAPVSSTSSLPLAALNMRVMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 4 - protein, 555 aa, synthetic construct
  source 1..555: note = TnpB-v3
MAGGPGAGSAAPVSSTSSLPLAALNMMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 5 - protein, 529 aa, synthetic construct
  source 1..529: note = Wild-type Cas12f1
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 6 - DNA, 1671 bp, synthetic construct
  source 1..1671: mol_type = other DNA; note = Human codon-optimized TnpB
atgggggagaaaagttcccgccgccgacggaatggaaaaagcggtgcgtggactgctgctataacaagctgtgttgggggtaagatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 7 - DNA, 1666 bp, synthetic construct
  source 1..1666: mol_type = other DNA; note = Human codon-optimized TnpB-V1
atggaaaagagaatcaacaagatcaggaagaagctgagcgccgacaacgccaccaagcctgtgtctaggagtggcccccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 8 - DNA, 1671 bp, synthetic construct
  source 1..1671: mol_type = other DNA; note = Human codon-optimized TnpB-V2
atggctggcggaccaggcgcaggtagtgctgcgccagtttcttcaacttcctccctgcccctggctgcgcttaacatgcgcgtgatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 9 - DNA, 1665 bp, synthetic construct
  source 1..1665: mol_type = other DNA; note = Human codon-optimized TnpB-V3
atggctggcggaccaggcgcaggtagtgctgcgccagtttcttcaacttcctccctgcccctggctgcgcttaacatgatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 10 - DNA, 1587 bp, synthetic construct
  source 1..1587: mol_type = other DNA; note = Human codon-optimized Cas12f1
atggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 11 - RNA, 161 nt, synthetic construct
  source 1..161: mol_type = other RNA; note = Wild-type tracrRNA for TnpB or Cas12f1 gRNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaa

SEQ ID NO: 12 - RNA, 37 nt, synthetic construct
  source 1..37: mol_type = other RNA; note = Wild-type crRNA for TnpB or Cas12f1 gRNA
gttgcagaacccgaatagacgaatgaaggaatgcaac

SEQ ID NO: 13 - RNA, 222 nt, synthetic construct
  source 1..222: mol_type = other RNA; note = Canonical sgRNA for TnpB or Cas12f1
  misc_feature 203..222: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacnnnnnnnnnnnnnnnnnnnn

SEQ ID NO: 14 - RNA, 20 nt, synthetic construct
  source 1..20: mol_type = other RNA; note = Xa of Formula (I)
cttcactgataaagtggaga

SEQ ID NO: 15 - RNA, 19 nt, synthetic construct
  source 1..19: mol_type = other RNA; note = Xa of Formula (I)
ttcactgataaagtggaga

SEQ ID NO: 16 - RNA, 18 nt, synthetic construct
  source 1..18: mol_type = other RNA; note = Xa of Formula (I)
tcactgataaagtggaga

SEQ ID NO: 17 - RNA, 17 nt, synthetic construct
  source 1..17: mol_type = other RNA; note = Xa of Formula (I)
cactgataaagtggaga

SEQ ID NO: 18 - RNA, 16 nt, synthetic construct
  source 1..16: mol_type = other RNA; note = Xa of Formula (I)
actgataaagtggaga

SEQ ID NO: 19 - RNA, 15 nt, synthetic construct
  source 1..15: mol_type = other RNA; note = Xa of Formula (I)
ctgataaagtggaga

SEQ ID NO: 20 - RNA, 14 nt, synthetic construct
  source 1..14: mol_type = other RNA; note = Xa of Formula (I)
tgataaagtggaga

SEQ ID NO: 21 - RNA, 13 nt, synthetic construct
  source 1..13: mol_type = other RNA; note = Xa of Formula (I)
gataaagtggaga

SEQ ID NO: 22 - RNA, 12 nt, synthetic construct
  source 1..12: mol_type = other RNA; note = Xa of Formula (I)
ataaagtggaga

SEQ ID NO: 23 - RNA, 11 nt, synthetic construct
  source 1..11: mol_type = other RNA; note = Xa of Formula (I)
taaagtggaga

SEQ ID NO: 24 - RNA, 10 nt, synthetic construct
  source 1..10: mol_type = other RNA; note = Xa of Formula (I)
aaagtggaga

SEQ ID NO: 25 - RNA, 13 nt, synthetic construct
  source 1..13: mol_type = other RNA; note = Xb1 of Formula (I)
caaaagctgtccc

SEQ ID NO: 26 - RNA, 12 nt, synthetic construct
  source 1..12: mol_type = other RNA; note = Xb1 of Formula (I)
caaaagctgtcc

SEQ ID NO: 27 - RNA, 11 nt, synthetic construct
  source 1..11: mol_type = other RNA; note = Xb1 of Formula (I)
caaaagctgtc

SEQ ID NO: 28 - RNA, 10 nt, synthetic construct
  source 1..10: mol_type = other RNA; note = Xb1 of Formula (I)
caaaagctgt

SEQ ID NO: 29 - RNA, 14 nt, synthetic construct
  source 1..14: mol_type = other RNA; note = Xb2 of Formula (I)
gggattagaacttg

SEQ ID NO: 30 - RNA, 13 nt, synthetic construct
  source 1..13: mol_type = other RNA; note = Xb2 of Formula (I)
ggattagaacttg

SEQ ID NO: 31 - RNA, 12 nt, synthetic construct
  source 1..12: mol_type = other RNA; note = Xb2 of Formula (I)
gattagaacttg

SEQ ID NO: 32 - RNA, 11 nt, synthetic construct
  source 1..11: mol_type = other RNA; note = Xb2 of Formula (I)
attagaacttg

SEQ ID NO: 33 - RNA, 10 nt, synthetic construct
  source 1..10: mol_type = other RNA; note = Xb2 of Formula (I)
ttagaacttg

SEQ ID NO: 34 - RNA, 31 nt, synthetic construct
  source 1..31: mol_type = other RNA; note = Xb1-UUAG-Xb2 of Formula (I)
caaaagctgtcccttaggggattagaacttg

SEQ ID NO: 35 - RNA, 25 nt, synthetic construct
  source 1..25: mol_type = other RNA; note = Xb1-UUAG-Xb2 of Formula (I)
caaaagctgtttagattagaacttg

SEQ ID NO: 36 - RNA, 20 nt, synthetic construct
  source 1..20: mol_type = other RNA; note = Xb1-UUAG-Xb2 of Formula (I)
caaaagctttagagaacttg

SEQ ID NO: 37 - RNA, 18 nt, synthetic construct
  source 1..18: mol_type = other RNA; note = Xb1-UUAG-Xb2 of Formula (I)
caaaagcttaggaacttg

SEQ ID NO: 38 - RNA, 10 nt, synthetic construct
  source 1..10: mol_type = other RNA; note = Xb1-UUAG-Xb2 of Formula (I)
caattagttg

SEQ ID NO: 39 - RNA, 28 nt, synthetic construct
  source 1..28: mol_type = other RNA; note = Xc1 of Formula (I)
ttcatttttcctctccaattctgcacaa

SEQ ID NO: 40 - RNA, 27 nt, synthetic construct
  source 1..27: mol_type = other RNA; note = Xc1 of Formula (I)
ttcatttttcctctccaattctgcaca

SEQ ID NO: 41 - RNA, 26 nt, synthetic construct
  source 1..26: mol_type = other RNA; note = Xc1 of Formula (I)
ttcatttttcctctccaattctgcac

SEQ ID NO: 42 - RNA, 25 nt, synthetic construct
  source 1..25: mol_type = other RNA; note = Xc1 of Formula (I)
ttcatttttcctctccaattctgca

SEQ ID NO: 43 - RNA, 24 nt, synthetic construct
  source 1..24: mol_type = other RNA; note = Xc1 of Formula (I)
ttcatttttcctctccaattctgc

SEQ ID NO: 44 - RNA, 23 nt, synthetic construct
  source 1..23: mol_type = other RNA; note = Xc1 of Formula (I)
ttcatttttcctctccaattctg

SEQ ID NO: 45 - RNA, 22 nt, synthetic construct
  source 1..22: mol_type = other RNA; note = Xc1 of Formula (I)
ttcatttttcctctccaattct

SEQ ID NO: 46 - RNA, 21 nt, synthetic construct
  source 1..21: mol_type = other RNA; note = Xc1 of Formula (I)
ttcatttttcctctccaattc

SEQ ID NO: 47 - RNA, 20 nt, synthetic construct
  source 1..20: mol_type = other RNA; note = Xc1 of Formula (I)
ttcatttttcctctccaatt

SEQ ID NO: 48 - RNA, 19 nt, synthetic construct
  source 1..19: mol_type = other RNA; note = Xc1 of Formula (I)
ttcatttttcctctccaat

SEQ ID NO: 49 - RNA, 18 nt, synthetic construct
  source 1..18: mol_type = other RNA; note = Xc1 of Formula (I)
ttcatttttcctctccaa

SEQ ID NO: 50 - RNA, 17 nt, synthetic construct
  source 1..17: mol_type = other RNA; note = Xc1 of Formula (I)
ttcatttttcctctcca

SEQ ID NO: 51 - RNA, 16 nt, synthetic construct
  source 1..16: mol_type = other RNA; note = Xc1 of Formula (I)
ttcatttttcctctcc

SEQ ID NO: 52 - RNA, 15 nt, synthetic construct
  source 1..15: mol_type = other RNA; note = Xc1 of Formula (I)
ttcatttttcctctc

SEQ ID NO: 53 - RNA, 14 nt, synthetic construct
  source 1..14: mol_type = other RNA; note = Xc1 of Formula (I)
ttcatttttcctct

SEQ ID NO: 54 - RNA, 13 nt, synthetic construct
  source 1..13: mol_type = other RNA; note = Xc1 of Formula (I)
ttcatttttcctc

SEQ ID NO: 55 - RNA, 12 nt, synthetic construct
  source 1..12: mol_type = other RNA; note = Xc1 of Formula (I)
ttcatttttcct

SEQ ID NO: 56 - RNA, 11 nt, synthetic construct
  source 1..11: mol_type = other RNA; note = Xc1 of Formula (I)
ttcatttttcc

SEQ ID NO: 57 - RNA, 10 nt, synthetic construct
  source 1..10: mol_type = other RNA; note = Xc1 of Formula (I)
ttcatttttc

SEQ ID NO: 58 - RNA, 27 nt, synthetic construct
  source 1..27: mol_type = other RNA; note = Xc2 of Formula (I)
gttgcagaacccgaatagacgaatgaa

SEQ ID NO: 59 - RNA, 26 nt, synthetic construct
  source 1..26: mol_type = other RNA; note = Xc2 of Formula (I)
ttgcagaacccgaatagacgaatgaa

SEQ ID NO: 60 - RNA, 25 nt, synthetic construct
  source 1..25: mol_type = other RNA; note = Xc2 of Formula (I)
tgcagaacccgaatagacgaatgaa

SEQ ID NO: 61 - RNA, 24 nt, synthetic construct
  source 1..24: mol_type = other RNA; note = Xc2 of Formula (I)
gcagaacccgaatagacgaatgaa

SEQ ID NO: 62 - RNA, 23 nt, synthetic construct
  source 1..23: mol_type = other RNA; note = Xc2 of Formula (I)
cagaacccgaatagacgaatgaa

SEQ ID NO: 63 - RNA, 22 nt, synthetic construct
  source 1..22: mol_type = other RNA; note = Xc2 of Formula (I)
agaacccgaatagacgaatgaa

SEQ ID NO: 64 - RNA, 21 nt, synthetic construct
  source 1..21: mol_type = other RNA; note = Xc2 of Formula (I)
gaacccgaatagacgaatgaa

SEQ ID NO: 65 - RNA, 20 nt, synthetic construct
  source 1..20: mol_type = other RNA; note = Xc2 of Formula (I)
aacccgaatagacgaatgaa

SEQ ID NO: 66 - RNA, 19 nt, synthetic construct
  source 1..19: mol_type = other RNA; note = Xc2 of Formula (I)
acccgaatagacgaatgaa

SEQ ID NO: 67 - RNA, 18 nt, synthetic construct
  source 1..18: mol_type = other RNA; note = Xc2 of Formula (I)
cccgaatagacgaatgaa

SEQ ID NO: 68 - RNA, 17 nt, synthetic construct
  source 1..17: mol_type = other RNA; note = Xc2 of Formula (I)
ccgaatagacgaatgaa

SEQ ID NO: 69 - RNA, 16 nt, synthetic construct
  source 1..16: mol_type = other RNA; note = Xc2 of Formula (I)
cgaatagacgaatgaa

SEQ ID NO: 70 - RNA, 15 nt, synthetic construct
  source 1..15: mol_type = other RNA; note = Xc2 of Formula (I)
gaatagacgaatgaa

SEQ ID NO: 71 - RNA, 14 nt, synthetic construct
  source 1..14: mol_type = other RNA; note = Xc2 of Formula (I)
aatagacgaatgaa

SEQ ID NO: 72 - RNA, 13 nt, synthetic construct
  source 1..13: mol_type = other RNA; note = Xc2 of Formula (I)
atagacgaatgaa

SEQ ID NO: 73 - RNA, 12 nt, synthetic construct
  source 1..12: mol_type = other RNA; note = Xc2 of Formula (I)
tagacgaatgaa

SEQ ID NO: 74 - RNA, 11 nt, synthetic construct
  source 1..11: mol_type = other RNA; note = Xc2 of Formula (I)
agacgaatgaa

SEQ ID NO: 75 - RNA, 10 nt, synthetic construct
  source 1..10: mol_type = other RNA; note = Xc2 of Formula (I)
gacgaatgaa

SEQ ID NO: 76 - RNA, 10 nt, synthetic construct
  source 1..10: mol_type = other RNA; note = Linker (Lk)
ttcgaaagaa

SEQ ID NO: 77 - RNA, 12 nt, synthetic construct
  source 1..12: mol_type = other RNA; note = Linker (Lk)
ttcagaaatgaa

SEQ ID NO: 78 - RNA, 14 nt, synthetic construct
  source 1..14: mol_type = other RNA; note = Linker (Lk)
ttcatgaaaatgaa

SEQ ID NO: 79 - RNA, 16 nt, synthetic construct
  source 1..16: mol_type = other RNA; note = Linker (Lk)
ttcattgaaaaatgaa

SEQ ID NO: 80 - RNA, 59 nt, synthetic construct
  source 1..59: mol_type = other RNA; note = Xc1-Lk-Xc2 of Formula (I)
ttcagtgctcctctccaattctgcacaagaaagttgcagaacccgaatagagcaatgaa

SEQ ID NO: 81 - RNA, 46 nt, synthetic construct
  source 1..46: mol_type = other RNA; note = Xc1-Lk-Xc2 of Formula (I)
ttcagtgctcctctccaattcgaaagaacccgaatagagcaatgaa

SEQ ID NO: 82 - RNA, 34 nt, synthetic construct
  source 1..34: mol_type = other RNA; note = Xc1-Lk-Xc2 of Formula (I)
ttcagtgctcctctcgaaagaatagagcaatgaa

SEQ ID NO: 83 - RNA, 22 nt, synthetic construct
  source 1..22: mol_type = other RNA; note = Xc1-Lk-Xc2 of Formula (I)
ttcagtgctgaaaagcaatgaa

SEQ ID NO: 84 - RNA, 18 nt, synthetic construct
  source 1..18: mol_type = other RNA; note = Xc1-Lk-Xc2 of Formula (I)
ttcatttgaaagaatgaa

SEQ ID NO: 85 - RNA, 14 nt, synthetic construct
  source 1..14: mol_type = other RNA; note = Xc1-Lk-Xc2 of Formula (I)
ttcatgaaaatgaa

SEQ ID NO: 86 - RNA, 10 nt, synthetic construct
  source 1..10: mol_type = other RNA; note = Xc1-Lk-Xc2 of Formula (I)
ttcgaaagaa

SEQ ID NO: 87 - RNA, 161 nt, synthetic construct
  source 1..161: mol_type = other RNA; note = Engineered tracrRNA for TnpB or Cas12f1 gRNA (MS1)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaa

SEQ ID NO: 88 - RNA, 154 nt, synthetic construct
  source 1..154: mol_type = other RNA; note = Engineered tracrRNA for TnpB or Cas12f1 gRNA (MS1/MS3-1)
gataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaa

SEQ ID NO: 89 - RNA, 147 nt, synthetic construct
  source 1..147: mol_type = other RNA; note = Engineered tracrRNA for TnpB or Cas12f1 gRNA (MS1/MS3-2)
tggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaa

SEQ ID NO: 90 - RNA, 141 nt, synthetic construct
  source 1..141: mol_type = other RNA; note = Engineered tracrRNA for TnpB or Cas12f1 gRNA (MS1/MS3-3)
accgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaa

SEQ ID NO: 91 - RNA, 154 nt, synthetic construct
  source 1..154: mol_type = other RNA; note = Engineered tracrRNA for TnpB or Cas12f1 gRNA (MS1/MS4*-1)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattc

SEQ ID NO: 92 - RNA, 148 nt, synthetic construct
  source 1..148: mol_type = other RNA; note = Engineered tracrRNA for TnpB or Cas12f1 gRNA (MS1/MS4*-2)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctc

SEQ ID NO: 93 - RNA, 142 nt, synthetic construct
  source 1..142: mol_type = other RNA; note = Engineered tracrRNA for TnpB or Cas12f1 gRNA (MS1/MS4*-3)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgct

SEQ ID NO: 94 - RNA, 155 nt, synthetic construct
  source 1..155: mol_type = other RNA; note = Engineered tracrRNA for TnpB or Cas12f1 gRNA (MS1/MS5-1)
cttcactgataaagtggagaaccgcttcaccaaaagctgtttagattagaacttgagtgaaggtgggctgcttgcatcagcctaatgtcgagaagtgctttcttcggaaagtaaccctcgaaacaaattcagtgctcctctccaattctgcacaa

SEQ ID NO: 95 - RNA, 148 nt, synthetic construct
  source 1..148: mol_type = other RNA; note = Engineered tracrRNA for TnpB or Cas12f1 gRNA (MS1/MS5-2)
cttcactgataaagtggagaaccgcttcaccaaaagcttaggaacttgagtgaaggtgggctgcttgcatcagcctaatgtcgagaagtgctttcttcggaaagtaaccctcgaaacaaattcagtgctcctctccaattctgcacaa

SEQ ID NO: 96 - RNA, 140 nt, synthetic construct
  source 1..140: mol_type = other RNA; note = Engineered tracrRNA for TnpB or Cas12f1 gRNA (MS1/MS5-3)
cttcactgataaagtggagaaccgcttcaccaattagttgagtgaaggtgggctgcttgcatcagcctaatgtcgagaagtgctttcttcggaaagtaaccctcgaaacaaattcagtgctcctctccaattctgcacaa

SEQ ID NO: 97 - RNA, 134 nt, synthetic construct
  source 1..134: mol_type = other RNA; note = Engineered tracrRNA for TnpB or Cas12f1 gRNA (MS1/MS3-3/MS4*-1)
accgcttcaccaaaagctgtcccttaggggattagaacttgagtgaaggtgggctgcttgcatcagcctaatgtcgagaagtgctttcttcggaaagtaaccctcgaaacaaattcagtgctcctctccaattc

SEQ ID NO: 98 - RNA, 128 nt, synthetic construct
  source 1..128: mol_type = other RNA; note = Engineered tracrRNA for TnpB or Cas12f1 gRNA (MS1/MS3-3/MS4*-2)
accgcttcaccaaaagctgtcccttaggggattagaacttgagtgaaggtgggctgcttgcatcagcctaatgtcgagaagtgctttcttcggaaagtaaccctcgaaacaaattcagtgctcctctc

SEQ ID NO: 99 - RNA, 122 nt, synthetic construct
  source 1..122: mol_type = other RNA; note = Engineered tracrRNA for TnpB or Cas12f1 gRNA (MS1/MS3-3/MS4*-3)
accgcttcaccaaaagctgtcccttaggggattagaacttgagtgaaggtgggctgcttgcatcagcctaatgtcgagaagtgctttcttcggaaagtaaccctcgaaacaaattcagtgct

SEQ ID NO: 100 - RNA, 142 nt, synthetic construct
  source 1..142: mol_type = other RNA; note = Engineered tracrRNA for TnpB or Cas12f1 gRNA (MS1/MS4*-2/MS5-1)
cttcactgataaagtggagaaccgcttcaccaaaagctgtttagattagaacttgagtgaaggtgggctgcttgcatcagcctaatgtcgagaagtgctttcttcggaaagtaaccctcgaaacaaattcagtgctcctctc

SEQ ID NO: 101 - RNA, 135 nt, synthetic construct
  source 1..135: mol_type = other RNA; note = Engineered tracrRNA for TnpB or Cas12f1 gRNA (MS1/MS4*-2/MS5-2)
cttcactgataaagtggagaaccgcttcaccaaaagcttaggaacttgagtgaaggtgggctgcttgcatcagcctaatgtcgagaagtgctttcttcggaaagtaaccctcgaaacaaattcagtgctcctctc

SEQ ID NO: 102 - RNA, 127 nt, synthetic construct
  source 1..127: mol_type = other RNA; note = Engineered tracrRNA for TnpB or Cas12f1 gRNA (MS1/MS4*-2/MS5-3)
cttcactgataaagtggagaaccgcttcaccaattagttgagtgaaggtgggctgcttgcatcagcctaatgtcgagaagtgctttcttcggaaagtaaccctcgaaacaaattcagtgctcctctc

SEQ ID NO: 103 - RNA, 135 nt, synthetic construct
  source 1..135: mol_type = other RNA; note = Engineered tracrRNA for TnpB or Cas12f1 gRNA (MS1/MS3-3/MS5-1)
accgcttcaccaaaagctgtttagattagaacttgagtgaaggtgggctgcttgcatcagcctaatgtcgagaagtgctttcttcggaaagtaaccctcgaaacaaattcagtgctcctctccaattctgcacaa

SEQ ID NO: 104 - RNA, 128 nt, synthetic construct
  source 1..128: mol_type = other RNA; note = Engineered tracrRNA for TnpB or Cas12f1 gRNA (MS1/MS3-3/MS5-2)
accgcttcaccaaaagcttaggaacttgagtgaaggtgggctgcttgcatcagcctaatgtcgagaagtgctttcttcggaaagtaaccctcgaaacaaattcagtgctcctctccaattctgcacaa

SEQ ID NO: 105 - RNA, 120 nt, synthetic construct
  source 1..120: mol_type = other RNA; note = Engineered tracrRNA for TnpB or Cas12f1 gRNA (MS1/MS3-3/MS5-3)
accgcttcaccaattagttgagtgaaggtgggctgcttgcatcagcctaatgtcgagaagtgctttcttcggaaagtaaccctcgaaacaaattcagtgctcctctccaattctgcacaa

SEQ ID NO: 106 - RNA, 107 nt, synthetic construct
  source 1..107: mol_type = other RNA; note = Engineered tracrRNA for TnpB or Cas12f1 gRNA (MS1/MS3-3/MS4*-2/MS5-3)
accgcttcaccaattagttgagtgaaggtgggctgcttgcatcagcctaatgtcgagaagtgctttcttcggaaagtaaccctcgaaacaaattcagtgctcctctc

SEQ ID NO: 107 - RNA, 140 nt, synthetic construct
  source 1..140: mol_type = other RNA; note = Engineered tracrRNA for TnpB or Cas12f1 gRNA (MF)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcattt

SEQ ID NO: 108 - RNA, 133 nt, synthetic construct
  source 1..133: mol_type = other RNA; note = Engineered tracrRNA for TnpB or Cas12f1 gRNA (MF/MS3-1)
gataaagtggagaaccgcttcaccaaaagctgtcccttaggggattagaacttgagtgaaggtgggctgcttgcatcagcctaatgtcgagaagtgctttcttcggaaagtaaccctcgaaacaaattcattt

SEQ ID NO: 109 - RNA, 126 nt, synthetic construct
  source 1..126: mol_type = other RNA; note = Engineered tracrRNA for TnpB or Cas12f1 gRNA (MF/MS3-2)
tggagaaccgcttcaccaaaagctgtcccttaggggattagaacttgagtgaaggtgggctgcttgcatcagcctaatgtcgagaagtgctttcttcggaaagtaaccctcgaaacaaattcattt

SEQ ID NO: 110 - RNA, 120 nt, synthetic construct
  source 1..120: mol_type = other RNA; note = Engineered tracrRNA for TnpB or Cas12f1 gRNA (MF/MS3-3)
accgcttcaccaaaagctgtcccttaggggattagaacttgagtgaaggtgggctgcttgcatcagcctaatgtcgagaagtgctttcttcggaaagtaaccctcgaaacaaattcattt

SEQ ID NO: 111 - RNA, 138 nt, synthetic construct
  source 1..138: mol_type = other RNA; note = Engineered tracrRNA for TnpB or Cas12f1 gRNA (MF/MS4-1)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcat

SEQ ID NO: 112 - RNA, 136 nt, synthetic construct
  source 1..136: mol_type = other RNA; note = Engineered tracrRNA for TnpB or Cas12f1 gRNA (MF/MS4-2)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattc

SEQ ID NO: 113 - RNA, 133 nt, synthetic construct
  source 1..133: mol_type = other RNA; note = Engineered tracrRNA for TnpB or Cas12f1 gRNA (MF/MS4-3)
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaa

SEQ ID NO: 114 - RNA, 134 nt, synthetic construct
  source 1..134: mol_type = other RNA; note = Engineered tracrRNA for TnpB or Cas12f1 gRNA (MF/MS5-1)
cttcactgataaagtggagaaccgcttcaccaaaagctgtttagattagaacttgagtgaaggtgggctgcttgcatcagcctaatgtcgagaagtgctttcttcggaaagtaaccctcgaaacaaattcattt

SEQ ID NO: 115 - RNA, 129 nt, synthetic construct
  source 1..129: mol_type = other RNA; note = Engineered tracrRNA for TnpB or Cas12f1 gRNA (MF/MS5-2)
cttcactgataaagtggagaaccgcttcaccaaaagctttagagaacttgagtgaaggtgggctgcttgcatcagcctaatgtcgagaagtgctttcttcggaaagtaaccctcgaaacaaattcattt

SEQ ID NO: 116 - RNA, 119 nt, synthetic construct
  source 1..119: mol_type = other RNA; note = Engineered tracrRNA for TnpB or Cas12f1 gRNA (MF/MS5-3)
cttcactgataaagtggagaaccgcttcaccaattagttgagtgaaggtgggctgcttgcatcagcctaatgtcgagaagtgctttcttcggaaagtaaccctcgaaacaaattcattt

SEQ ID NO: 117 - RNA, 113 nt, synthetic construct
  source 1..113: mol_type = other RNA; note = Engineered tracrRNA for TnpB or Cas12f1 gRNA (MF/MS5)
cttcactgataaagtggagaaccgcttcacttagagtgaaggtgggctgcttgcatcagcctaatgtcgagaagtgctttcttcggaaagtaaccctcgaaacaaattcattt

SEQ ID NO: 118 - RNA, 118 nt, synthetic construct
  source 1..118: mol_type = other RNA; note = Engineered tracrRNA for TnpB or Cas12f1 gRNA (MF/MS3-3/MS4-1)
accgcttcaccaaaagctgtcccttaggggattagaacttgagtgaaggtgggctgcttgcatcagcctaatgtcgagaagtgctttcttcggaaagtaaccctcgaaacaaattcat

SEQ ID NO: 119 - RNA, 116 nt, synthetic construct
  source 1..116: mol_type = other RNA; note = Engineered tracrRNA for TnpB or Cas12f1 gRNA (MF/MS3-3/MS4-2)
accgcttcaccaaaagctgtcccttaggggattagaacttgagtgaaggtgggctgcttgcatcagcctaatgtcgagaagtgctttcttcggaaagtaaccctcgaaacaaattc

SEQ ID NO: 120 - RNA, 113 nt, synthetic construct
  source 1..113: mol_type = other RNA; note = Engineered tracrRNA for TnpB or Cas12f1 gRNA (MF/MS3-3/MS4-3)
accgcttcaccaaaagctgtcccttaggggattagaacttgagtgaaggtgggctgcttgcatcagcctaatgtcgagaagtgctttcttcggaaagtaaccctcgaaacaaa

SEQ ID NO: 121 - RNA, 127 nt, synthetic construct
  source 1..127: mol_type = other RNA; note = Engineered tracrRNA for TnpB or Cas12f1 gRNA (MF/MS4-3/MS5-1)
cttcactgataaagtggagaaccgcttcaccaaaagctgtttagattagaacttgagtgaaggtgggctgcttgcatcagcctaatgtcgagaagtgctttcttcggaaagtaaccctcgaaacaaa

SEQ ID NO: 122 - RNA, 122 nt, synthetic construct
  source 1..122: mol_type = other RNA; note = Engineered tracrRNA for TnpB or Cas12f1 gRNA (MF/MS4-3/MS5-2)
cttcactgataaagtggagaaccgcttcaccaaaagctttagagaacttgagtgaaggtgggctgcttgcatcagcctaatgtcgagaagtgctttcttcggaaagtaaccctcgaaacaaa

SEQ ID NO: 123 - RNA, 112 nt, synthetic construct
  source 1..112: mol_type = other RNA; note = Engineered tracrRNA for TnpB or Cas12f1 gRNA (MF/MS4-3/MS5-3)
cttcactgataaagtggagaaccgcttcaccaattagttgagtgaaggtgggctgcttgcatcagcctaatgtcgagaagtgctttcttcggaaagtaaccctcgaaacaaa

SEQ ID NO: 124 - RNA, 106 nt, synthetic construct
  source 1..106: mol_type = other RNA; note = Engineered tracrRNA for TnpB or Cas12f1 gRNA (MF/MS4-3/MS5)
cttcactgataaagtggagaaccgcttcacttagagtgaaggtgggctgcttgcatcagcctaatgtcgagaagtgctttcttcggaaagtaaccctcgaaacaaa

SEQ ID NO: 125 - RNA, 114 nt, synthetic construct
  source 1..114: mol_type = other RNA; note = Engineered tracrRNA for TnpB or Cas12f1 gRNA (MF/MS3-3/MS5-1)
accgcttcaccaaaagctgtttagattagaacttgagtgaaggtgggctgcttgcatcagcctaatgtcgagaagtgctttcttcggaaagtaaccctcgaaacaaattcattt

SEQ ID NO: 126 - RNA, 109 nt, synthetic construct
  source 1..109: mol_type = other RNA; note = Engineered tracrRNA for TnpB or Cas12f1 gRNA (MF/MS3-3/MS5-2)
accgcttcaccaaaagctttagagaacttgagtgaaggtgggctgcttgcatcagcctaatgtcgagaagtgctttcttcggaaagtaaccctcgaaacaaattcattt

SEQ ID NO: 127 - RNA, 99 nt, synthetic construct
  source 1..99: mol_type = other RNA; note = Engineered tracrRNA for TnpB or Cas12f1 gRNA (MF/MS3-3/MS5-3)
accgcttcaccaattagttgagtgaaggtgggctgcttgcatcagcctaatgtcgagaagtgctttcttcggaaagtaaccctcgaaacaaattcattt

SEQ ID NO: 128 - RNA, 93 nt, synthetic construct
  source 1..93: mol_type = other RNA; note = Engineered tracrRNA for TnpB or Cas12f1 gRNA (MF/MS3-3/MS5)
accgcttcacttagagtgaaggtgggctgcttgcatcagcctaatgtcgagaagtgctttcttcggaaagtaaccctcgaaacaaattcattt

SEQ ID NO: 129 - RNA, 92 nt, synthetic construct
  source 1..92: mol_type = other RNA; note = Engineered tracrRNA for TnpB or Cas12f1 gRNA (MF/MS3-3/MS4-3/MS5-3)
accgcttcaccaattagttgagtgaaggtgggctgcttgcatcagcctaatgtcgagaagtgctttcttcggaaagtaaccctcgaaacaaa

SEQ ID NO: 130 - RNA, 91 nt, synthetic construct
  source 1..91: mol_type = other RNA; note = Engineered tracrRNA for TnpB or Cas12f1 gRNA (MF/MS3-3/MS4-1/MS5)
accgcttcacttagagtgaaggtgggctgcttgcatcagcctaatgtcgagaagtgctttcttcggaaagtaaccctcgaaacaaattcat

SEQ ID NO: 131 - RNA, 89 nt, synthetic construct
  source 1..89: mol_type = other RNA; note = Engineered tracrRNA for TnpB or Cas12f1 gRNA (MF/MS3-3/MS4-2/MS5)
accgcttcacttagagtgaaggtgggctgcttgcatcagcctaatgtcgagaagtgctttcttcggaaagtaaccctcgaaacaaattc

SEQ ID NO: 132 - RNA, 86 nt, synthetic construct
  source 1..86: mol_type = other RNA; note = Engineered tracrRNA for TnpB or Cas12f1 gRNA (MF/MS3-3/MS4-3/MS5)
accgcttcacttagagtgaaggtgggctgcttgcatcagcctaatgtcgagaagtgctttcttcggaaagtaaccctcgaaacaaa

SEQ ID NO: 133 - RNA, 37 nt, synthetic construct
  source 1..37: mol_type = other RNA; note = Engineered crRNA for TnpB or Cas12f1 gRNA (MS1)
gttgcagaacccgaatagagcaatgaaggaatgcaac

SEQ ID NO: 134 - RNA, 31 nt, synthetic construct
  source 1..31: mol_type = other RNA; note = Engineered crRNA for TnpB or Cas12f1 gRNA (MS1/MS4*-1)
gaacccgaatagagcaatgaaggaatgcaac

SEQ ID NO: 135 - RNA, 25 nt, synthetic construct
  source 1..25: mol_type = other RNA; note = Engineered crRNA for TnpB or Cas12f1 gRNA (MS1/MS4*-2)
gaatagagcaatgaaggaatgcaac

SEQ ID NO: 136 - RNA, 19 nt, synthetic construct
  source 1..19: mol_type = other RNA; note = Engineered crRNA for TnpB or Cas12f1 gRNA (MS1/MS4*-3)
agcaatgaaggaatgcaac

SEQ ID NO: 137 - RNA, 17 nt, synthetic construct
  source 1..17: mol_type = other RNA; note = Engineered crRNA for TnpB or Cas12f1 gRNA (MF)
gaatgaaggaatgcaac

SEQ ID NO: 138 - RNA, 15 nt, synthetic construct
  source 1..15: mol_type = other RNA; note = Engineered crRNA for TnpB or Cas12f1 gRNA (MF/MS4-1)
atgaaggaatgcaac

SEQ ID NO: 139 - RNA, 13 nt, synthetic construct
  source 1..13: mol_type = other RNA; note = Engineered crRNA for TnpB or Cas12f1 gRNA (MF/MS4-2)
gaaggaatgcaac

SEQ ID NO: 140 - RNA, 10 nt, synthetic construct
  source 1..10: mol_type = other RNA; note = Engineered crRNA for TnpB or Cas12f1 gRNA (MF/MS4-3)
ggaatgcaac

SEQ ID NO: 141 - RNA, 68 nt, synthetic construct
  source 1..68: mol_type = other RNA; note = Engineered crRNA for TnpB or Cas12f1 gRNA (MS1/MS2)
  misc_feature 38..57: note = n is a, c, g or u
gttgcagaacccgaatagagcaatgaaggaatgcaacnnnnnnnnnnnnnnnnnnnnttttatttttt

SEQ ID NO: 142 - RNA, 62 nt, synthetic construct
  source 1..62: mol_type = other RNA; note = Engineered crRNA for TnpB or Cas12f1 gRNA (MS1/MS2/MS4*-1)
  misc_feature 32..51: note = n is a, c, g or u
gaacccgaatagagcaatgaaggaatgcaacnnnnnnnnnnnnnnnnnnnnttttatttttt

SEQ ID NO: 143 - RNA, 56 nt, synthetic construct
  source 1..56: mol_type = other RNA; note = Engineered crRNA for TnpB or Cas12f1 gRNA (MS1/MS2/MS4*-2)
  misc_feature 26..45: note = n is a, c, g or u
gaatagagcaatgaaggaatgcaacnnnnnnnnnnnnnnnnnnnnttttatttttt

SEQ ID NO: 144 - RNA, 50 nt, synthetic construct
  source 1..50: mol_type = other RNA; note = Engineered crRNA for TnpB or Cas12f1 gRNA (MS1/MS2/MS4*-3)
  misc_feature 20..39: note = n is a, c, g or u
agcaatgaaggaatgcaacnnnnnnnnnnnnnnnnnnnnttttatttttt

SEQ ID NO: 145 - RNA, 48 nt, synthetic construct
  source 1..48: mol_type = other RNA; note = Engineered crRNA for TnpB or Cas12f1 gRNA (MF/MS2)
  misc_feature 18..37: note = n is a, c, g or u
gaatgaaggaatgcaacnnnnnnnnnnnnnnnnnnnnttttatttttt

SEQ ID NO: 146 - RNA, 46 nt, synthetic construct
  source 1..46: mol_type = other RNA; note = Engineered crRNA for TnpB or Cas12f1 gRNA (MF/MS2/MS4-1)
  misc_feature 16..35: note = n is a, c, g or u
atgaaggaatgcaacnnnnnnnnnnnnnnnnnnnnttttatttttt

SEQ ID NO: 147 - RNA, 44 nt, synthetic construct
  source 1..44: mol_type = other RNA; note = Engineered crRNA for TnpB or Cas12f1 gRNA (MF/MS2/MS4-2)
  misc_feature 14..33: note = n is a, c, g or u
gaaggaatgcaacnnnnnnnnnnnnnnnnnnnnttttatttttt

SEQ ID NO: 148 - RNA, 41 nt, synthetic construct
  source 1..41: mol_type = other RNA; note = Engineered crRNA for TnpB or Cas12f1 gRNA (MF/MS2/MS4-3)
  misc_feature 11..30: note = n is a, c, g or u
ggaatgcaacnnnnnnnnnnnnnnnnnnnnttttatttttt

SEQ ID NO: 149 - RNA, 222 nt, synthetic construct
  source 1..222: mol_type = other RNA; note = Engineered sgRNA with modification of MS1 for TnpB or Cas12f1
  misc_feature 203..222: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 150 - RNA, 233 nt, synthetic construct
  source 1..233: mol_type = other RNA; note = Engineered sgRNA with modification of MS1/MS2 for TnpB or Cas12f1
  misc_feature 203..222: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnnttttatttttt

SEQ ID NO: 151 - RNA, 213 nt, synthetic construct
  source 1..213: mol_type = other RNA; note = Engineered sgRNA with modification of MS1/MS2/MS3 for TnpB or Cas12f1
  misc_structure 183..202: note = n is a, c, g or u
accgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnnttttatttttt

SEQ ID NO: 152 - RNA, 158 nt, synthetic construct
  source 1..158: mol_type = other RNA; note = Engineered sgRNA with modification of MS2/MS3/MS4 for TnpB or Cas12f1
  misc_feature 128..147: note = n is a, c, g or u
accgcttcaccaaaagctgtcccttaggggattagaacttgagtgaaggtgggctgcttgcatcagcctaatgtcgagaagtgctttcttcggaaagtaaccctcgaaacaaagaaaggaatgcaacnnnnnnnnnnnnnnnnnnnnttttatttttt

SEQ ID NO: 153 - RNA, 131 nt, synthetic construct
  source 1..131: mol_type = other RNA; note = Engineered sgRNA with modification of MS2/MS3/MS4/MS5 for TnpB or Cas12f1
  misc_feature 99..118: note = n is a, c, g or u
accgcttcacttagagtgaaggtgggctgcttgcatcagcctaatgtcgagaagtgctttcttcggaaagtaaccctcgaaacaaagaaaggaatgcaacnnnnnnnnnnnnnnnnnnnnttttatttttt

SEQ ID NO: 154 - RNA, 215 nt, synthetic construct
  source 1..215: mol_type = other RNA; note = Engineered sgRNA with modification of MS1/MS3-1 for TnpB or Cas12f1
  misc_feature 196..215: note = n is a, c, g or u
gataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 155 - RNA, 208 nt, synthetic construct
  source 1..208: mol_type = other RNA; note = Engineered sgRNA with modification of MS1/MS3-2 for TnpB or Cas12f1
  misc_feature 189..208: note = n is a, c, g or u
tggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 156 - RNA, 202 nt, synthetic construct
  source 1..202: mol_type = other RNA; note = Engineered sgRNA with modification of MS1/MS3-3 for TnpB or Cas12f1
  misc_feature 183..202: note = n is a, c, g or u
accgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 157 - RNA, 209 nt, synthetic construct
  source 1..209: mol_type = other RNA; note = Engineered sgRNA with modification of MS1/MS4*-1 for TnpB or Cas12f1
  misc_feature 190..209: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctccaattcgaaagaacccgaatagagcaatgaaggaatgcaacnnnnnnnnnnnnnnnnnnnn

SEQ ID NO: 158 - RNA, 197 nt, synthetic construct
  source 1..197: mol_type = other RNA; note = Engineered sgRNA with modification of MS1/MS4*-2 for TnpB or Cas12f1
  misc_feature 178..197: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnn

SEQ ID NO: 159 - RNA, 185 nt, synthetic construct
  source 1..185: mol_type = other RNA; note = Engineered sgRNA with modification of MS1/MS4*-3 for TnpB or Cas12f1
  misc_feature 166..185: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctgaaaagcaatgaaggaatgcaacnnnnnnnnnnnnnnnnnnnn

SEQ ID NO: 160 - RNA, 216 nt, synthetic construct
  source 1..216: mol_type = other RNA; note = Engineered sgRNA with modification of MS1/MS5-1 for TnpB or Cas12f1
  misc_feature 197..216: note = n is a, c, g or u
cttcactgataaagtggagaaccgcttcaccaaaagctgtttagattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 161 - RNA, 209 nt, synthetic construct
  source 1..209: mol_type = other RNA; note = Engineered sgRNA with modification of MS1/MS5-2 for TnpB or Cas12f1
  misc_feature 190..209: note = n is a, c, g or u
cttcactgataaagtggagaaccgcttcaccaaaagcttaggaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 162 - RNA, 201 nt, synthetic construct
  source 1..201: mol_type = other RNA; note = Engineered sgRNA with modification of MS1/MS5-3 for TnpB or Cas12f1
  misc_feature 182..201: note = n is a, c, g or u
cttcactgataaagtggagaaccgcttcaccaattagttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 163 - RNA, 206 nt, synthetic construct
  source 1..206: mol_type = other RNA; note = Engineered sgRNA with modification of MS1/MS2/MS4*-2 for TnpB or Cas12f1
  misc_feature 178..197: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnnttttatttt

SEQ ID NO: 164 - RNA, 177 nt, synthetic construct
  source 1..177: mol_type = other RNA; note = Engineered sgRNA with modification of MS1/MS3-3/MS4*-2 for TnpB or Cas12f1
  misc_feature 158..177: note = n is a, c, g or u
accgcttcaccaaaagctgtcccttaggggattagaacttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnn

SEQ ID NO: 165 - RNA, 210 nt, synthetic construct
  source 1..210: mol_type = other RNA; note = Engineered sgRNA with modification of MS1/MS2/MS5-3 for TnpB or Cas12f1
  misc_feature 182..201: note = n is a, c, g or u
cttcactgataaagtggagaaccgcttcaccaattagttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnnttttatttt

SEQ ID NO: 166 - RNA, 181 nt, synthetic construct
  source 1..181: mol_type = other RNA; note = Engineered sgRNA with modification of MS1/MS3-3/MS5-3 for TnpB or Cas12f1
  misc_feature 162..181: note = n is a, c, g or u
accgcttcaccaattagttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnn

SEQ ID NO: 167 - RNA, 176 nt, synthetic construct
  source 1..176: mol_type = other RNA; note = Engineered sgRNA with modification of MS1/MS4*-2/MS5-3 for TnpB or Cas12f1
  misc_feature 157..176: note = n is a, c, g or u
cttcactgataaagtggagaaccgcttcaccaattagttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnn

SEQ ID NO: 168 - RNA, 186 nt, synthetic construct
  source 1..186: mol_type = other RNA; note = Engineered sgRNA with modification of MS1/MS2/MS3-3/MS4*-2 for TnpB or Cas12f1
  misc_feature 158..177: note = n is a, c, g or u
accgcttcaccaaaagctgtcccttaggggattagaacttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnnttttatttt

SEQ ID NO: 169 - RNA, 190 nt, synthetic construct
  source 1..190: mol_type = other RNA; note = Engineered sgRNA with modification of MS1/MS2/MS3-3/MS5-3 for TnpB or Cas12f1
  misc_feature 162..181: note = n is a, c, g or u
accgcttcaccaattagttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaacnnnnnnnnnnnnnnnnnnnnttttatttt

SEQ ID NO: 170 - RNA, 185 nt, synthetic construct
  source 1..185: mol_type = other RNA; note = Engineered sgRNA with modification of MS1/MS2/MS4*-2/MS5-3 for TnpB or Cas12f1
  misc_feature 157..176: note = n is a, c, g or u
cttcactgataaagtggagaaccgcttcaccaattagttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnnttttatttt

SEQ ID NO: 171 - RNA, 156 nt, synthetic construct
  source 1..156: mol_type = other RNA; note = Engineered sgRNA with modification of MS1/MS3-3/MS4*-2/MS5-3 for TnpB or Cas12f1
  misc_feature 137..156: note = n is a, c, g or u
accgcttcaccaattagttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnn

SEQ ID NO: 172 - RNA, 165 nt, synthetic construct
  source 1..165: mol_type = other RNA; note = Engineered sgRNA with modification of MS1/MS2/MS3-3/MS4*-2/MS5-3 for TnpB or Cas12f1
  misc_feature 137..156: note = n is a, c, g or u
accgcttcaccaattagttgagtgaaggtgggctgcttgcatcagcctaatgtcgagaagtgctttcttcggaaagtaaccctcgaaacaaattcagtgctcctctcgaaagaatagagcaatgaaggaatgcaacnnnnnnnnnnnnnnnnnnnnttttatttt

SEQ ID NO: 173 - RNA, 181 nt, synthetic construct
  source 1..181: mol_type = other RNA; note = Mature form sgRNA for TnpB or Cas12f1
  misc_feature 162..181: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 174 - RNA, 174 nt, synthetic construct
  source 1..174: mol_type = other RNA; note = Mature form sgRNA with modification of MS3-1 for TnpB or Cas12f1
  misc_feature 155..174: note = n is a, c, g or u
gataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 175 - RNA, 167 nt, synthetic construct
  source 1..167: mol_type = other RNA; note = Mature form sgRNA with modification of MS3-2 for TnpB or Cas12f1
  misc_feature 148..167: note = n is a, c, g or u
tggagaaccgcttcaccaaaagctgtcccttaggggattagaacttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 176 - RNA, 161 nt, synthetic construct
  source 1..161: mol_type = other RNA; note = Mature form sgRNA with modification of MS3-3 for TnpB or Cas12f1
  misc_feature 142..161: note = n is a, c, g or u
accgcttcaccaaaagctgtcccttaggggattagaacttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 177 - RNA, 177 nt, synthetic construct
  source 1..177: mol_type = other RNA; note = Mature form sgRNA with modification of MS4-1 for TnpB or Cas12f1
  misc_feature 158..177: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatgaaaatgaaggaatgcaacnnnnnnnnnnnnnnnnnnnn

SEQ ID NO: 178 - RNA, 173 nt, synthetic construct
  source 1..173: mol_type = other RNA; note = Mature form sgRNA with modification of MS4-2 for TnpB or Cas12f1
  misc_feature 154..173: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcgaaagaaggaatgcaacnnnnnnnnnnnnnnnnnnnn

SEQ ID NO: 179 - RNA, 167 nt, synthetic construct
  source 1..167: mol_type = other RNA; note = Mature form sgRNA with modification of MS4-3 for TnpB or Cas12f1
  misc_feature 148..167: note = n is a, c, g or u
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaagaaaggaatgcaacnnnnnnnnnnnnnnnnnnnn

SEQ ID NO: 180 - RNA, 175 nt, synthetic construct
  source 1..175: mol_type = other RNA; note = Mature form sgRNA with modification of MS5-1 for TnpB or Cas12f1
  misc_feature 156..175: note = n is a, c, g or u
cttcactgataaagtggagaaccgcttcaccaaaagctgtttagattagaacttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 181 - RNA, 170 nt, synthetic construct
  source 1..170: mol_type = other RNA; note = Mature form sgRNA with modification of MS5-2 for TnpB or Cas12f1
  misc_feature 151..170: note = n is a, c, g or u
cttcactgataaagtggagaaccgcttcaccaaaagctttagagaacttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 182 - RNA, 160 nt, synthetic construct
  source 1..160: mol_type = other RNA; note = Mature form sgRNA with modification of MS5-3 for TnpB or Cas12f1
  misc_feature 141..160: note = n is a, c, g or u
cttcactgataaagtggagaaccgcttcaccaattagttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 183 - RNA, 147 nt, synthetic construct
  source 1..147: mol_type = other RNA; note = Mature form sgRNA with modification of MS3-3/MS4-3 for TnpB or Cas12f1
  misc_feature 128..147: note = n is a, c, g or u
accgcttcaccaaaagctgtcccttaggggattagaacttgagtgaaggtgggctgcttgcatcagcctaatgtcgagaagtgctttcttcggaaagtaaccctcgaaacaaagaaaggaatgcaacnnnnnnnnnnnnnnnnnnnn

SEQ ID NO: 184 - RNA, 140 nt, synthetic construct
  source 1..140: mol_type = other RNA; note = Mature form sgRNA with modification of MS3-3/MS5-3 for TnpB or Cas12f1
  misc_feature 121..140: note = n is a, c, g or u
accgcttcaccaattagttgagtgaaggtgggctgcttgcatcagcctaatgtcgagaagtgctttcttcggaaagtaaccctcgaaacaaattcatttgaaagaatgaaggaatgcaacnnnnnnnnnnnnnnnnnnnn

SEQ ID NO: 185 - RNA, 146 nt, synthetic construct
  source 1..146: mol_type = other RNA; note = Mature form sgRNA with modification of MS4-3/MS5-3 for TnpB or Cas12f1
  misc_feature 127..146: note = n is a, c, g or u
cttcactgataaagtggagaaccgcttcaccaattagttgagtgaaggtgggctgcttgcatcagcctaatgtcgagaagtgctttcttcggaaagtaaccctcgaaacaaagaaaggaatgcaacnnnnnnnnnnnnnnnnnnnn

SEQ ID NO: 186 - RNA, 126 nt, synthetic construct
  source 1..126: mol_type = other RNA; note = Mature form sgRNA with modification of MS3-3/MS4-3/MS5-3 for TnpB or Cas12f1
  misc_feature 107..126: note = n is a, c, g or u
accgcttcaccaattagttgagtgaaggtgggctgcttgcatcagcctaatgtcgagaagtgctttcttcggaaagtaaccctcgaaacaaagaaaggaatgcaacnnnnnnnnnnnnnnnnnnnn

SEQ ID NO: 187 - RNA, 10 nt, synthetic construct
  source 1..10: mol_type = other RNA; note = The additional sequence for TnpB or Cas12f1 gRNA
ataaaggtga

SEQ ID NO: 188 - RNA, 37 nt, synthetic construct
  source 1..37: mol_type = other RNA; note = The additional sequence for TnpB or Cas12f1 gRNA
ctgatgagtccgtgaggacgaaacgagtaagctcgtc

SEQ ID NO: 189 - RNA, 37 nt, synthetic construct
  source 1..37: mol_type = other RNA; note = The additional sequence for TnpB or Cas12f1 gRNA
ctgctcgaatgagcaaagcaggagtgcctgagtagtc

SEQ ID NO: 190 - DNA, 20 bp, synthetic construct
  source 1..20: mol_type = other DNA; note = F102 -protospacer
aggttgtctcctcattagag

SEQ ID NO: 191 - DNA, 20 bp, synthetic construct
  source 1..20: mol_type = other DNA; note = F104 -protospacer
atattcttagaatcgttcac

SEQ ID NO: 192 - DNA, 20 bp, synthetic construct
  source 1..20: mol_type = other DNA; note = F108 -protospacer
ccaccaatcttctggttata

SEQ ID NO: 193 - DNA, 20 bp, synthetic construct
  source 1..20: mol_type = other DNA; note = F109 -protospacer
tagacaacagactcaagagc

SEQ ID NO: 194 - DNA, 20 bp, synthetic construct
  source 1..20: mol_type = other DNA; note = F110 -protospacer
actgactactccccaagtat

SEQ ID NO: 195 - DNA, 20 bp, synthetic construct
  source 1..20: mol_type = other DNA; note = F111 -protospacer
gggccttatctccagtttct

SEQ ID NO: 196 - DNA, 20 bp, synthetic construct
  source 1..20: mol_type = other DNA; note = F114 -protospacer
taaacgcctggctagtaaga

SEQ ID NO: 197 - DNA, 20 bp, synthetic construct
  source 1..20: mol_type = other DNA; note = F115 -protospacer
taaaaacagtaatacctaac

SEQ ID NO: 198 - DNA, 20 bp, synthetic construct
  source 1..20: mol_type = other DNA; note = F118 -protospacer
ccttgcttactgcttattgc

SEQ ID NO: 199 - DNA, 20 bp, synthetic construct
  source 1..20: mol_type = other DNA; note = F119 -protospacer
ttcagtactagcaataagca

SEQ ID NO: 200 - DNA, 20 bp, synthetic construct
  source 1..20: mol_type = other DNA; note = F121 -protospacer
tttaatgactttgaaacagt

SEQ ID NO: 201 - DNA, 20 bp, synthetic construct
  source 1..20: mol_type = other DNA; note = F123 -protospacer
aaacagtatttcatgtctaa

SEQ ID NO: 202 - DNA, 20 bp, synthetic construct
  source 1..20: mol_type = other DNA; note = F124 -protospacer
gacatgaaatactgtttcaa

SEQ ID NO: 203 - DNA, 20 bp, synthetic construct
  source 1..20: mol_type = other DNA; note = F128 -protospacer
agaaaatattgtatcttggt

SEQ ID NO: 204 - DNA, 20 bp, synthetic construct
  source 1..20: mol_type = other DNA; note = F130 -protospacer
tttcagattgttagtaaact

SEQ ID NO: 205 - DNA, 20 bp, synthetic construct
  source 1..20: mol_type = other DNA; note = F131 -protospacer
tactgggagcagtttatcat

SEQ ID NO: 206 - DNA, 20 bp, synthetic construct
  source 1..20: mol_type = other DNA; note = F133 -protospacer
atttccctagggtccagctt

SEQ ID NO: 207 - DNA, 20 bp, synthetic construct
  source 1..20: mol_type = other DNA; note = F134 -protospacer
aagctggaccctagggaaat

SEQ ID NO: 208 - DNA, 20 bp, synthetic construct
  source 1..20: mol_type = other DNA; note = F135 -protospacer
aaattcccttgaataggaag

SEQ ID NO: 209 - DNA, 20 bp, synthetic construct
  source 1..20: mol_type = other DNA; note = F136 -protospacer
aatcagaaagaagatcttat

SEQ ID NO: 210 - DNA, 20 bp, synthetic construct
  source 1..20: mol_type = other DNA; note = F138 -protospacer
ttcaagaaaaaacaaaggca

SEQ ID NO: 211 - DNA, 20 bp, synthetic construct
  source 1..20: mol_type = other DNA; note = F140 -protospacer
ccacttccacaatgtatatg

SEQ ID NO: 212 - DNA, 20 bp, synthetic construct
  source 1..20: mol_type = other DNA; note = F141 -protospacer
acttaagttacttgtccagg

SEQ ID NO: 213 - DNA, 20 bp, synthetic construct
  source 1..20: mol_type = other DNA; note = F142 -protospacer
ctcattctcatgcctggaca

SEQ ID NO: 214 - DNA, 20 bp, synthetic construct
  source 1..20: mol_type = other DNA; note = F143 -protospacer
aaaaattgttaaatgtatat

SEQ ID NO: 215 - DNA, 20 bp, synthetic construct
  source 1..20: mol_type = other DNA; note = F147 -protospacer
attgaagagtaacaatttga

SEQ ID NO: 216 - DNA, 20 bp, synthetic construct
  source 1..20: mol_type = other DNA; note = F148 -protospacer
acttattgttattgaaattg

SEQ ID NO: 217 - DNA, 20 bp, synthetic construct
  source 1..20: mol_type = other DNA; note = F43 -protospacer
gtatcaattcacaccagcaa

SEQ ID NO: 218 - DNA, 20 bp, synthetic construct
  source 1..20: mol_type = other DNA; note = R52 -protospacer
ctctcctagaccatttccca

SEQ ID NO: 219 - DNA, 20 bp, synthetic construct
  source 1..20: mol_type = other DNA; note = R55 -protospacer
ctgagagagaaacagttgcc

SEQ ID NO: 220 - DNA, 20 bp, synthetic construct
  source 1..20: mol_type = other DNA; note = R56 -protospacer
tatccttgattatacttagg

SEQ ID NO: 221 - DNA, 20 bp, synthetic construct
  source 1..20: mol_type = other DNA; note = R57 -protospacer
cattaatttatatccttgat

SEQ ID NO: 222 - DNA, 20 bp, synthetic construct
  source 1..20: mol_type = other DNA; note = R58 -protospacer
ttatttgcattaatttatat

SEQ ID NO: 223 - DNA, 20 bp, synthetic construct
  source 1..20: mol_type = other DNA; note = R59 -protospacer
actgagacaactattcttgt

SEQ ID NO: 224 - DNA, 20 bp, synthetic construct
  source 1..20: mol_type = other DNA; note = R60 -protospacer
gtaaatttaactgagacaac

SEQ ID NO: 225 - DNA, 20 bp, synthetic construct
  source 1..20: mol_type = other DNA; note = R61 -protospacer
aaataccaggttgtttagta

SEQ ID NO: 226 - DNA, 20 bp, synthetic construct
  source 1..20: mol_type = other DNA; note = R62 -protospacer
taccaaataagtcactcaac

SEQ ID NO: 227 - DNA, 20 bp, synthetic construct
  source 1..20: mol_type = other DNA; note = R63 -protospacer
aatgtaaatagctcagttga

SEQ ID NO: 228 - DNA, 20 bp, synthetic construct
  source 1..20: mol_type = other DNA; note = R64 -protospacer
gcactacgcagcccaacata

SEQ ID NO: 229 - DNA, 20 bp, synthetic construct
  source 1..20: mol_type = other DNA; note = R65 -protospacer
attctgcaatatgttgggct

SEQ ID NO: 230 - DNA, 20 bp, synthetic construct
  source 1..20: mol_type = other DNA; note = R66 -protospacer
ccttctccaatgataaggat

SEQ ID NO: 231 - DNA, 20 bp, synthetic construct
  source 1..20: mol_type = other DNA; note = R68 -protospacer
agttatagctctctttcaat

SEQ ID NO: 232 - DNA, 20 bp, synthetic construct
  source 1..20: mol_type = other DNA; note = R69 -protospacer
aaattaccctagatcttaaa

SEQ ID NO: 233 - DNA, 20 bp, synthetic construct
  source 1..20: mol_type = other DNA; note = R70 -protospacer
tgcaatgccatgttcaaatg

SEQ ID NO: 234 - DNA, 20 bp, synthetic construct
  source 1..20: mol_type = other DNA; note = R71 -protospacer
aacatggcattgcataaatg

SEQ ID NO: 235 - DNA, 20 bp, synthetic construct
  source 1..20: mol_type = other DNA; note = R73 -protospacer
ccaaatggattacactgagg

SEQ ID NO: 236 - DNA, 20 bp, synthetic construct
  source 1..20: mol_type = other DNA; note = R74 -protospacer
gtaaaattatgacatcaact

SEQ ID NO: 237 - DNA, 20 bp, synthetic construct
  source 1..20: mol_type = other DNA; note = R75 -protospacer
cctagctagatcaaatatac

SEQ ID NO: 238 - DNA, 20 bp, synthetic construct
  source 1..20: mol_type = other DNA; note = R76 -protospacer
atctagctaggtaaaccata

SEQ ID NO: 239 - DNA, 20 bp, synthetic construct
  source 1..20: mol_type = other DNA; note = R77 -protospacer
tatttgtgaatgattaagaa

SEQ ID NO: 240 - DNA, 20 bp, synthetic construct
  source 1..20: mol_type = other DNA; note = R79 -protospacer
atgtataacaattccaacat

SEQ ID NO: 241 - DNA, 20 bp, synthetic construct
  source 1..20: mol_type = other DNA; note = R82 -protospacer
gaaaggcttgaaagctgtta

SEQ ID NO: 242 - DNA, 20 bp, synthetic construct
  source 1..20: mol_type = other DNA; note = R84 -protospacer
tatttctttagaaaggcttg

SEQ ID NO: 243 - DNA, 20 bp, synthetic construct
  source 1..20: mol_type = other DNA; note = R85 -protospacer
atacctaaataccttcagca

SEQ ID NO: 244 - DNA, 20 bp, synthetic construct
  source 1..20: mol_type = other DNA; note = R86 -protospacer
aaaaaacaagaagtgaggca

SEQ ID NO: 245 - DNA, 20 bp, synthetic construct
  source 1..20: mol_type = other DNA; note = R89 -protospacer
taaaataaacttcaccaatt

SEQ ID NO: 246 - DNA, 20 bp, synthetic construct
  source 1..20: mol_type = other DNA; note = R90 -protospacer
caaagaaattccccttactc

SEQ ID NO: 247 - DNA, 20 bp, synthetic construct
  source 1..20: mol_type = other DNA; note = R91 -protospacer
ttcctatggaattggtgaag

SEQ ID NO: 248 - DNA, 20 bp, synthetic construct
  source 1..20: mol_type = other DNA; note = R92 -protospacer
aaggccttgagcttgaatac

SEQ ID NO: 249 - DNA, 20 bp, synthetic construct
  source 1..20: mol_type = other DNA; note = R93 -protospacer
aatgcaggttgtttactatc

SEQ ID NO: 250 - DNA, 20 bp, synthetic construct
  source 1..20: mol_type = other DNA; note = R94 -protospacer
caaatgatgattgtggtcaa

SEQ ID NO: 251 - DNA, 20 bp, synthetic construct
  source 1..20: mol_type = other DNA; note = R95 -protospacer
caaatggcactgaattcagg

SEQ ID NO: 252 - DNA, 20 bp, synthetic construct
  source 1..20: mol_type = other DNA; note = R97 -protospacer
tatacatccatatccataac

SEQ ID NO: 253 - DNA, 20 bp, synthetic construct
  source 1..20: mol_type = other DNA; note = R98 -protospacer
catatatttatatacatcca

SEQ ID NO: 254 - DNA, 20 bp, synthetic construct
  source 1..20: mol_type = other DNA; note = R99 -protospacer
cttctgctttaaaaaaagta

SEQ ID NO: 255 - DNA, 20 bp, synthetic construct
  source 1..20: mol_type = other DNA; note = F1 -protospacer
tcatcccaatggcatattta

SEQ ID NO: 256 - DNA, 20 bp, synthetic construct
  source 1..20: mol_type = other DNA; note = F2 -protospacer
gtcatcatgaaccattctct

SEQ ID NO: 257 - DNA, 20 bp, synthetic construct
  source 1..20: mol_type = other DNA; note = F3 -protospacer
agtctcaggccctggccttg

SEQ ID NO: 258 - DNA, 20 bp, synthetic construct
  source 1..20: mol_type = other DNA; note = F4 -protospacer
gaaaaagacagaaaggaaga

SEQ ID NO: 259 - DNA, 20 bp, synthetic construct
  source 1..20: mol_type = other DNA; note = F5 -protospacer
ttgaaatttagtgtgacatg

SEQ ID NO: 260 - DNA, 20 bp, synthetic construct
  source 1..20: mol_type = other DNA; note = F6 -protospacer
gtgtgacatgatcatttcca

SEQ ID NO: 261 - DNA, 20 bp, synthetic construct
  source 1..20: mol_type = other DNA; note = F7 -protospacer
accctcaaataagagcagag

SEQ ID NO: 262 - DNA, 20 bp, synthetic construct
  source 1..20: mol_type = other DNA; note = F8 -protospacer
agggtcaaattttcaataag

SEQ ID NO: 263 - DNA, 20 bp, synthetic construct
  source 1..20: mol_type = other DNA; note = F9 -protospacer
aaaagtgacttattgaaaat

SEQ ID NO: 264 - DNA, 20 bp, synthetic construct
  source 1..20: mol_type = other DNA; note = F10 -protospacer
aaaaagcattttctgacact

SEQ ID NO: 265 - DNA, 20 bp, synthetic construct
  source 1..20: mol_type = other DNA; note = F11 -protospacer
ggaggatttcaagcttatgg

SEQ ID NO: 266 - DNA, 20 bp, synthetic construct
  source 1..20: mol_type = other DNA; note = F12 -protospacer
ggaacactccatccacattt

SEQ ID NO: 267 - DNA, 20 bp, synthetic construct
  source 1..20: mol_type = other DNA; note = F13 -protospacer
ccagtcagcctggtgttctg

SEQ ID NO: 268 - DNA, 20 bp, synthetic construct
  source 1..20: mol_type = other DNA; note = F14 -protospacer
atttagctctcttttcatcc

SEQ ID NO: 269 - DNA, 20 bp, synthetic construct
  source 1..20: mol_type = other DNA; note = F15 -protospacer
gctctcttttcatcctcaca

SEQ ID NO: 270 - DNA, 20 bp, synthetic construct
  source 1..20: mol_type = other DNA; note = F16 -protospacer
gctcctttcctggaatccct

SEQ ID NO: 271 - DNA, 20 bp, synthetic construct
  source 1..20: mol_type = other DNA; note = F17 -protospacer
cttccatgatttcttctcct

SEQ ID NO: 272 - DNA, 20 bp, synthetic construct
  source 1..20: mol_type = other DNA; note = F18 -protospacer
tgtcacagtccaggagaaga

SEQ ID NO: 273 - DNA, 20 bp, synthetic construct
  source 1..20: mol_type = other DNA; note = F19 -protospacer
cattacatttaattctttct

SEQ ID NO: 274 - DNA, 20 bp, synthetic construct
  source 1..20: mol_type = other DNA; note = F20 -protospacer
attctttctagaaagagcct

SEQ ID NO: 275 - DNA, 20 bp, synthetic construct
  source 1..20: mol_type = other DNA; note = F21 -protospacer
tttctcaaagctatctgact

SEQ ID NO: 276 - DNA, 20 bp, synthetic construct
  source 1..20: mol_type = other DNA; note = F22 -protospacer
ttgcctcactgttactgcct

SEQ ID NO: 277 - DNA, 20 bp, synthetic construct
  source 1..20: mol_type = other DNA; note = F23 -protospacer
tgtcacgaaacaatgattga

SEQ ID NO: 278 - DNA, 20 bp, synthetic construct
  source 1..20: mol_type = other DNA; note = F24 -protospacer
attgtcagagagaataaaaa

SEQ ID NO: 279 - DNA, 20 bp, synthetic construct
  source 1..20: mol_type = other DNA; note = F25 -protospacer
ttctctctgacaattaaaac

SEQ ID NO: 280 - DNA, 20 bp, synthetic construct
  source 1..20: mol_type = other DNA; note = F26 -protospacer
atcaatgcagacagaaaaaa

SEQ ID NO: 281 - DNA, 20 bp, synthetic construct
  source 1..20: mol_type = other DNA; note = R1 -protospacer
acatctatgagcctcagtta

SEQ ID NO: 282 - DNA, 20 bp, synthetic construct
  source 1..20: mol_type = other DNA; note = R2 -protospacer
aatccttgttctgctactta

SEQ ID NO: 283 - DNA, 20 bp, synthetic construct
  source 1..20: mol_type = other DNA; note = R3 -protospacer
attcctaagcttgtgttatt

SEQ ID NO: 284 - DNA, 20 bp, synthetic construct
  source 1..20: mol_type = other DNA; note = R4 -protospacer
gtgatttgtatgtagatgta

SEQ ID NO: 285 - DNA, 20 bp, synthetic construct
  source 1..20: mol_type = other DNA; note = R5 -protospacer
tatgtagatgtagatgtagt

SEQ ID NO: 286 - DNA, 20 bp, synthetic construct
  source 1..20: mol_type = other DNA; note = R6 -protospacer
tggttgctatgtactgatac

SEQ ID NO: 287 - DNA, 20 bp, synthetic construct
  source 1..20: mol_type = other DNA; note = R7 -protospacer
caataccatattgagttata

SEQ ID NO: 288 - DNA, 20 bp, synthetic construct
  source 1..20: mol_type = other DNA; note = R8 -protospacer
gtaaataaaagtcctgggag

SEQ ID NO: 289 - DNA, 20 bp, synthetic construct
  source 1..20: mol_type = other DNA; note = R9 -protospacer
tttaccaaaggaaacaatat

SEQ ID NO: 290 - DNA, 20 bp, synthetic construct
  source 1..20: mol_type = other DNA; note = R10 -protospacer
ccaaaggaaacaatatttta

SEQ ID NO: 291 - DNA, 20 bp, synthetic construct
  source 1..20: mol_type = other DNA; note = R11 -protospacer
aacattataaaatattgttt

SEQ ID NO: 292 - DNA, 20 bp, synthetic construct
  source 1..20: mol_type = other DNA; note = R12 -protospacer
taatgtttaaagcccaggtt

SEQ ID NO: 293 - DNA, 20 bp, synthetic construct
  source 1..20: mol_type = other DNA; note = R13 -protospacer
aagcccaggttttgaagtta

SEQ ID NO: 294 - DNA, 20 bp, synthetic construct
  source 1..20: mol_type = other DNA; note = R14 -protospacer
aacccagacaatgtaacttc

SEQ ID NO: 295 - DNA, 20 bp, synthetic construct
  source 1..20: mol_type = other DNA; note = R15 -protospacer
aagttacattgtctgggttc

SEQ ID NO: 296 - DNA, 20 bp, synthetic construct
  source 1..20: mol_type = other DNA; note = R16 -protospacer
tagctagataaacttgggct

SEQ ID NO: 297 - DNA, 20 bp, synthetic construct
  source 1..20: mol_type = other DNA; note = R17 -protospacer
tctagctataaaatggggat

SEQ ID NO: 298 - DNA, 20 bp, synthetic construct
  source 1..20: mol_type = other DNA; note = R18 -protospacer
tcatcaggattaagttggtt

SEQ ID NO: 299 - DNA, 20 bp, synthetic construct
  source 1..20: mol_type = other DNA; note = R19 -protospacer
ttcaatactagcagtaagca

SEQ ID NO: 300 - DNA, 20 bp, synthetic construct
  source 1..20: mol_type = other DNA; note = R20 -protospacer
ccttgcttactgcttactgc

SEQ ID NO: 301 - DNA, 20 bp, synthetic construct
  source 1..20: mol_type = other DNA; note = R21 -protospacer
gttccaccacgaactctaga

SEQ ID NO: 302 - DNA, 20 bp, synthetic construct
  source 1..20: mol_type = other DNA; note = R22 -protospacer
cataatacaaatgccatcat

SEQ ID NO: 303 - DNA, 20 bp, synthetic construct
  source 1..20: mol_type = other DNA; note = R23 -protospacer
tattatgcaaactgtatatc

SEQ ID NO: 304 - DNA, 20 bp, synthetic construct
  source 1..20: mol_type = other DNA; note = R24 -protospacer
tgagtccagcatttagggaa

SEQ ID NO: 305 - DNA, 20 bp, synthetic construct
  source 1..20: mol_type = other DNA; note = R25 -protospacer
gggaagccattgatgtgctc

SEQ ID NO: 306 - DNA, 20 bp, synthetic construct
  source 1..20: mol_type = other DNA; note = R26 -protospacer
gagaactctggagactactg

SEQ ID NO: 307 - DNA, 20 bp, synthetic construct
  source 1..20: mol_type = other DNA; note = R27 -protospacer
tttagagaactctggagact

SEQ ID NO: 308 - DNA, 20 bp, synthetic construct
  source 1..20: mol_type = other DNA; note = R28 -protospacer
atgccccctcacagagatcg

SEQ ID NO: 309 - DNA, 20 bp, synthetic construct
  source 1..20: mol_type = other DNA; note = R29 -protospacer
aagacgatctctgtgagggg

SEQ ID NO: 310 - DNA, 20 bp, synthetic construct
  source 1..20: mol_type = other DNA; note = R30 -protospacer
acctatatttaaagacgatc

SEQ ID NO: 311 - DNA, 20 bp, synthetic construct
  source 1..20: mol_type = other DNA; note = R31 -protospacer
aatataggtcaaaaactaat

SEQ ID NO: 312 - RNA, 19 nt, synthetic construct
  source 1..19: mol_type = other RNA; note = Spacer 1 for dystrophin(w/o U-tail)
ctcattctcatgcctggac

SEQ ID NO: 313 - RNA, 20 nt, synthetic construct
  source 1..20: mol_type = other RNA; note = Spacer 2 for dystrophin(w/o U-tail)
ctcattctcatgcctggaca

SEQ ID NO: 314 - RNA, 21 nt, synthetic construct
  source 1..21: mol_type = other RNA; note = Spacer 3 for dystrophin(w/o U-tail)
ctcattctcatgcctggacaa

SEQ ID NO: 315 - RNA, 22 nt, synthetic construct
  source 1..22: mol_type = other RNA; note = Spacer 4 for dystrophin(w/o U-tail)
ctcattctcatgcctggacaag

SEQ ID NO: 316 - RNA, 24 nt, synthetic construct
  source 1..24: mol_type = other RNA; note = Spacer 5 for dystrophin(w/o U-tail)
ctcattctcatgcctggacaagta

SEQ ID NO: 317 - RNA, 25 nt, synthetic construct
  source 1..25: mol_type = other RNA; note = Spacer 6 for dystrophin(w/o U-tail)
ctcattctcatgcctggacaagtaa

SEQ ID NO: 318 - RNA, 19 nt, synthetic construct
  source 1..19: mol_type = other RNA; note = Spacer 7 for dystrophin(w/o U-tail)
ctcattctcatgcctggac

SEQ ID NO: 319 - RNA, 20 nt, synthetic construct
  source 1..20: mol_type = other RNA; note = Spacer 8 for dystrophin(w/o U-tail)
ctcattctcatgcctggaca

SEQ ID NO: 320 - RNA, 21 nt, synthetic construct
  source 1..21: mol_type = other RNA; note = Spacer 9 for dystrophin(w/o U-tail)
ctcattctcatgcctggacaa

SEQ ID NO: 321 - RNA, 22 nt, synthetic construct
  source 1..22: mol_type = other RNA; note = Spacer 10 for dystrophin(w/o U-tail)
ctcattctcatgcctggacaag

SEQ ID NO: 322 - RNA, 24 nt, synthetic construct
  source 1..24: mol_type = other RNA; note = Spacer 11 for dystrophin(w/o U-tail)
ctcattctcatgcctggacaagta

SEQ ID NO: 323 - RNA, 25 nt, synthetic construct
  source 1..25: mol_type = other RNA; note = Spacer 12 for dystrophin(w/o U-tail)
ctcattctcatgcctggacaagtaa

SEQ ID NO: 324 - RNA, 19 nt, synthetic construct
  source 1..19: mol_type = other RNA; note = Spacer 13 for dystrophin(w/o U-tail)
ctctcctagaccatttccc

SEQ ID NO: 325 - RNA, 20 nt, synthetic construct
  source 1..20: mol_type = other RNA; note = Spacer 14 for dystrophin(w/o U-tail)
ctctcctagaccatttccca

SEQ ID NO: 326 - RNA, 21 nt, synthetic construct
  source 1..21: mol_type = other RNA; note = Spacer 15 for dystrophin(w/o U-tail)
ctctcctagaccatttcccac

SEQ ID NO: 327 - RNA, 22 nt, synthetic construct
  source 1..22: mol_type = other RNA; note = Spacer 16 for dystrophin(w/o U-tail)
ctctcctagaccatttcccacc

SEQ ID NO: 328 - RNA, 23 nt, synthetic construct
  source 1..23: mol_type = other RNA; note = Spacer 17 for dystrophin(w/o U-tail)
ctctcctagaccatttcccacca

SEQ ID NO: 329 - RNA, 24 nt, synthetic construct
  source 1..24: mol_type = other RNA; note = Spacer 18 for dystrophin(w/o U-tail)
ctctcctagaccatttcccaccag

SEQ ID NO: 330 - RNA, 19 nt, synthetic construct
  source 1..19: mol_type = other RNA; note = Spacer 19 for dystrophin(w/o U-tail)
ctctcctagaccatttccc

SEQ ID NO: 331 - RNA, 20 nt, synthetic construct
  source 1..20: mol_type = other RNA; note = Spacer 20 for dystrophin(w/o U-tail)
ctctcctagaccatttccca

SEQ ID NO: 332 - RNA, 21 nt, synthetic construct
  source 1..21: mol_type = other RNA; note = Spacer 21 for dystrophin(w/o U-tail)
ctctcctagaccatttcccac

SEQ ID NO: 333 - RNA, 22 nt, synthetic construct
  source 1..22: mol_type = other RNA; note = Spacer 22 for dystrophin(w/o U-tail)
ctctcctagaccatttcccacc

SEQ ID NO: 334 - RNA, 23 nt, synthetic construct
  source 1..23: mol_type = other RNA; note = Spacer 23 for dystrophin(w/o U-tail)
ctctcctagaccatttcccacca

SEQ ID NO: 335 - RNA, 24 nt, synthetic construct
  source 1..24: mol_type = other RNA; note = Spacer 24 for dystrophin(w/o U-tail)
ctctcctagaccatttcccaccag

SEQ ID NO: 336 - RNA, 28 nt, synthetic construct
  source 1..28: mol_type = other RNA; note = Spacer 1 for dystrophin(w/ U-tail)
ctcattctcatgcctggacttttatttt

SEQ ID NO: 337 - RNA, 29 nt, synthetic construct
  source 1..29: mol_type = other RNA; note = Spacer 2 for dystrophin(w/ U-tail)
ctcattctcatgcctggacattttatttt

SEQ ID NO: 338 - RNA, 30 nt, synthetic construct
  source 1..30: mol_type = other RNA; note = Spacer 3 for dystrophin(w/ U-tail)
ctcattctcatgcctggacaattttatttt

SEQ ID NO: 339 - RNA, 31 nt, synthetic construct
  source 1..31: mol_type = other RNA; note = Spacer 4 for dystrophin(w/ U-tail)
ctcattctcatgcctggacaagttttatttt

SEQ ID NO: 340 - RNA, 33 nt, synthetic construct
  source 1..33: mol_type = other RNA; note = Spacer 5 for dystrophin(w/ U-tail)
ctcattctcatgcctggacaagtattttatttt

SEQ ID NO: 341 - RNA, 34 nt, synthetic construct
  source 1..34: mol_type = other RNA; note = Spacer 6 for dystrophin(w/ U-tail)
ctcattctcatgcctggacaagtaattttatttt

SEQ ID NO: 342 - RNA, 23 nt, synthetic construct
  source 1..23: mol_type = other RNA; note = Spacer 7 for dystrophin(w/ U-tail)
ctcattctcatgcctggactttt

SEQ ID NO: 343 - RNA, 24 nt, synthetic construct
  source 1..24: mol_type = other RNA; note = Spacer 8 for dystrophin(w/ U-tail)
ctcattctcatgcctggacatttt

SEQ ID NO: 344 - RNA, 25 nt, synthetic construct
  source 1..25: mol_type = other RNA; note = Spacer 9 for dystrophin(w/ U-tail)
ctcattctcatgcctggacaatttt

SEQ ID NO: 345 - RNA, 26 nt, synthetic construct
  source 1..26: mol_type = other RNA; note = Spacer 10 for dystrophin(w/ U-tail)
ctcattctcatgcctggacaagtttt

SEQ ID NO: 346 - RNA, 28 nt, synthetic construct
  source 1..28: mol_type = other RNA; note = Spacer 11 for dystrophin(w/ U-tail)
ctcattctcatgcctggacaagtatttt

SEQ ID NO: 347 - RNA, 29 nt, synthetic construct
  source 1..29: mol_type = other RNA; note = Spacer 12 for dystrophin(w/ U-tail)
ctcattctcatgcctggacaagtaatttt

SEQ ID NO: 348 - RNA, 28 nt, synthetic construct
  source 1..28: mol_type = other RNA; note = Spacer 13 for dystrophin(w/ U-tail)
ctctcctagaccatttcccttttatttt

SEQ ID NO: 349 - RNA, 29 nt, synthetic construct
  source 1..29: mol_type = other RNA; note = Spacer 14 for dystrophin(w/ U-tail)
ctctcctagaccatttcccattttatttt

SEQ ID NO: 350 - RNA, 30 nt, synthetic construct
  source 1..30: mol_type = other RNA; note = Spacer 15 for dystrophin(w/ U-tail)
ctctcctagaccatttcccacttttatttt

SEQ ID NO: 351 - RNA, 31 nt, synthetic construct
  source 1..31: mol_type = other RNA; note = Spacer 16 for dystrophin(w/ U-tail)
ctctcctagaccatttcccaccttttatttt

SEQ ID NO: 352 - RNA, 32 nt, synthetic construct
  source 1..32: mol_type = other RNA; note = Spacer 17 for dystrophin(w/ U-tail)
ctctcctagaccatttcccaccattttatttt

SEQ ID NO: 353 - RNA, 33 nt, synthetic construct
  source 1..33: mol_type = other RNA; note = Spacer 18 for dystrophin(w/ U-tail)
ctctcctagaccatttcccaccagttttatttt

SEQ ID NO: 354 - RNA, 23 nt, synthetic construct
  source 1..23: mol_type = other RNA; note = Spacer 19 for dystrophin(w/ U-tail)
ctctcctagaccatttccctttt

SEQ ID NO: 355 - RNA, 24 nt, synthetic construct
  source 1..24: mol_type = other RNA; note = Spacer 20 for dystrophin(w/ U-tail)
ctctcctagaccatttcccatttt

SEQ ID NO: 356 - RNA, 25 nt, synthetic construct
  source 1..25: mol_type = other RNA; note = Spacer 21 for dystrophin(w/ U-tail)
ctctcctagaccatttcccactttt

SEQ ID NO: 357 - RNA, 26 nt, synthetic construct
  source 1..26: mol_type = other RNA; note = Spacer 22 for dystrophin(w/ U-tail)
ctctcctagaccatttcccacctttt

SEQ ID NO: 358 - RNA, 27 nt, synthetic construct
  source 1..27: mol_type = other RNA; note = Spacer 23 for dystrophin(w/ U-tail)
ctctcctagaccatttcccaccatttt

SEQ ID NO: 359 - RNA, 28 nt, synthetic construct
  source 1..28: mol_type = other RNA; note = Spacer 24 for dystrophin(w/ U-tail)
ctctcctagaccatttcccaccagtttt

SEQ ID NO: 360 - RNA, 47 nt, synthetic construct
  source 1..47: mol_type = other RNA; note = shRNA for ATM1-1
ggagccagatagtttgtatttcaagagaatacaaactatctggctcc

SEQ ID NO: 361 - RNA, 47 nt, synthetic construct
  source 1..47: mol_type = other RNA; note = shRNA for ATM1-2
gcaagcagctgaaacaaatttcaagagaatttgtttcagctgcttgc

SEQ ID NO: 362 - RNA, 47 nt, synthetic construct
  source 1..47: mol_type = other RNA; note = shRNA for ATM1-3
ggagctgattgtagcaacattcaagagatgttgctacaatcagctcc

SEQ ID NO: 363 - RNA, 47 nt, synthetic construct
  source 1..47: mol_type = other RNA; note = shRNA for ATM1-4
gcacagaagtgcctccaatttcaagagaattggaggcacttctgtgc

SEQ ID NO: 364 - RNA, 47 nt, synthetic construct
  source 1..47: mol_type = other RNA; note = shRNA for ATM1-5
ggacatagtttctgggagattcaagagatctcccagaaactatgtcc

SEQ ID NO: 365 - RNA, 47 nt, synthetic construct
  source 1..47: mol_type = other RNA; note = shRNA for XRCC4-1
ggatgacactggcacattattcaagagataatgtgccagtgtcatcc

SEQ ID NO: 366 - RNA, 47 nt, synthetic construct
  source 1..47: mol_type = other RNA; note = shRNA for XRCC4-2
ggagagtactgatgaggaattcaagagattcctcatcagtactctcc

SEQ ID NO: 367 - RNA, 47 nt, synthetic construct
  source 1..47: mol_type = other RNA; note = shRNA for XRCC4-3
gaatccaccttgtttctgattcaagagatcagaaacaaggtggattc

SEQ ID NO: 368 - RNA, 47 nt, synthetic construct
  source 1..47: mol_type = other RNA; note = shRNA for XRCC4-4
gtacaagtatcttgggagattcaagagatctcccaagatacttgtac

SEQ ID NO: 369 - RNA, 47 nt, synthetic construct
  source 1..47: mol_type = other RNA; note = shRNA for XRCC4-5
gaatgcagctcaagaacgattcaagagatcgttcttgagctgcattc

SEQ ID NO: 370 - RNA, 47 nt, synthetic construct
  source 1..47: mol_type = other RNA; note = shRNA for XLF-1
gcatgagtctggcattacattcaagagatgtaatgccagactcatgc

SEQ ID NO: 371 - RNA, 47 nt, synthetic construct
  source 1..47: mol_type = other RNA; note = shRNA for XLF-2
gaaagccctttgtcatgaattcaagagattcatgacaaagggctttc

SEQ ID NO: 372 - RNA, 47 nt, synthetic construct
  source 1..47: mol_type = other RNA; note = shRNA for XLF-3
gaacagtgcttccctgcaattcaagagattgcagggaagcactgttc

SEQ ID NO: 373 - RNA, 47 nt, synthetic construct
  source 1..47: mol_type = other RNA; note = shRNA for XLF-4
ggaaagacctagagatccattcaagagatggatctctaggtctttcc

SEQ ID NO: 374 - RNA, 47 nt, synthetic construct
  source 1..47: mol_type = other RNA; note = shRNA for XLF-5
gtatggcagtcaccacacattcaagagatgtgtggtgactgccatac

SEQ ID NO: 375 - RNA, 47 nt, synthetic construct
  source 1..47: mol_type = other RNA; note = shRNA for XRCC6-1
gcagcattgtgcagatacattcaagagatgtatctgcacaatgctgc

SEQ ID NO: 376 - RNA, 47 nt, synthetic construct
  source 1..47: mol_type = other RNA; note = shRNA for XRCC6-2
gcaggaacatccctccttattcaagagataaggagggatgttcctgc

SEQ ID NO: 377 - RNA, 47 nt, synthetic construct
  source 1..47: mol_type = other RNA; note = shRNA for XRCC6-3
gcagtgctctgctcatcaattcaagagattgatgagcagagcactgc

SEQ ID NO: 378 - RNA, 47 nt, synthetic construct
  source 1..47: mol_type = other RNA; note = shRNA for XRCC6-4
ggatcatgctgttcaccaattcaagagattggtgaacagcatgatcc

SEQ ID NO: 379 - RNA, 47 nt, synthetic construct
  source 1..47: mol_type = other RNA; note = shRNA for XRCC6-5
ggatctgactactcactcattcaagagatgagtgagtagtcagatcc

SEQ ID NO: 380 - RNA, 47 nt, synthetic construct
  source 1..47: mol_type = other RNA; note = shRNA for LIG4-1
gcacaaagatggagatgtattcaagagatacatctccatctttgtgc

SEQ ID NO: 381 - RNA, 47 nt, synthetic construct
  source 1..47: mol_type = other RNA; note = shRNA for LIG4-2
gcagacacgtactgtgtaattcaagagattacacagtacgtgtctgc

SEQ ID NO: 382 - RNA, 47 nt, synthetic construct
  source 1..47: mol_type = other RNA; note = shRNA for LIG4-3
ggagcagactcctgaagaattcaagagattcttcaggagtctgctcc

SEQ ID NO: 383 - RNA, 47 nt, synthetic construct
  source 1..47: mol_type = other RNA; note = shRNA for LIG4-4
ggaggattctgatctgcaattcaagagattgcagatcagaatcctcc

SEQ ID NO: 384 - RNA, 47 nt, synthetic construct
  source 1..47: mol_type = other RNA; note = shRNA for LIG4-5
gcatgatccttctgtaggattcaagagatcctacagaaggatcatgc

SEQ ID NO: 385 - RNA, 47 nt, synthetic construct
  source 1..47: mol_type = other RNA; note = shRNA for DCLRE1C-1
ggagactcctacccagatattcaagagatatctgggtaggagtctcc

SEQ ID NO: 386 - RNA, 47 nt, synthetic construct
  source 1..47: mol_type = other RNA; note = shRNA for DCLRE1C-2
ggacaaagctgactacagattcaagagatctgtagtcagctttgtcc

SEQ ID NO: 387 - RNA, 47 nt, synthetic construct
  source 1..47: mol_type = other RNA; note = shRNA for DCLRE1C-3
gcagagctctcgtttcacattcaagagatgtgaaacgagagctctgc

SEQ ID NO: 388 - RNA, 47 nt, synthetic construct
  source 1..47: mol_type = other RNA; note = shRNA for DCLRE1C-4
ggactctgatggagaatcattcaagagatgattctccatcagagtcc

SEQ ID NO: 389 - RNA, 47 nt, synthetic construct
  source 1..47: mol_type = other RNA; note = shRNA for DCLRE1C-5
gcagaattcttcccagtcattcaagagatgactgggaagaattctgc

SEQ ID NO: 390 - protein, 249 aa, synthetic construct
  source 1..249: note = N-term 249aa extension
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQAL

SEQ ID NO: 391 - protein, 248 aa, synthetic construct
  source 1..248: note = C-term 248aa extension
RARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 392 - protein, 557 aa, synthetic construct
  source 1..557: note = Engineered CWCas12f1 (S170T)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLTDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 393 - protein, 557 aa, synthetic construct
  source 1..557: note = Engineered CWCas12f1 (S188Q)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIAQGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 394 - protein, 557 aa, synthetic construct
  source 1..557: note = Engineered CWCas12f1 (S188H)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIAHGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 395 - protein, 557 aa, synthetic construct
  source 1..557: note = Engineered CWCas12f1 (S188K)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIAKGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 396 - protein, 557 aa, synthetic construct
  source 1..557: note = Engineered CWCas12f1 (R191K)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLKSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 397 - protein, 557 aa, synthetic construct
  source 1..557: note = Engineered CWCas12f1 (Q225T)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKTKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 398 - protein, 557 aa, synthetic construct
  source 1..557: note = Engineered CWCas12f1 (Q225F)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKFKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 399 - protein, 557 aa, synthetic construct
  source 1..557: note = Engineered CWCas12f1 (Q272K)
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVKKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 400 - RNA, 22 nt, synthetic construct
  source 1..22: mol_type = other RNA; note = shRNA for Scrambled-3
cagagctaactcagatagtact

SEQ ID NO: 401 - DNA, 21 bp, synthetic construct
  source 1..21: mol_type = other DNA; note = F68-protospacer for SaCas9
gtgtattgcttgtactactca

SEQ ID NO: 402 - DNA, 21 bp, synthetic construct
  source 1..21: mol_type = other DNA; note = R84-protospacer for SaCas9
gtgttattacttgctactgca

SEQ ID NO: 403 - RNA, 47 nt, synthetic construct
  source 1..47: mol_type = other RNA; note = shRNA for ATM1-6
gaacttcagtggaccttcattcaagagatgaaggtccactgaagttc